(19) United States
(12) Patent Application Publication
(10) Pub. No.: US 2024/0191032 A1
(43) Pub. Date: Jun. 13, 2024

(54) MECHANOCHROMISM AND STRAIN-INDUCED CRYSTALLIZATION IN THIOL-YNE-DERIVED STEREOELASTOMERS

(71) Applicant: Duke University, Durham, NC (US)

(72) Inventors: Matthew BECKER, Chapel Hill, NC (US); Virginia RITTER, Chappaqua,, NY (US); Stephen CRAIG, Chapel Hill, NC (US)

(21) Appl. No.: 18/324,739

(22) Filed: May 26, 2023

Related U.S. Application Data
(60) Provisional application No. 63/346,432, filed on May 27, 2022.

(51) Int. Cl. C08G 75/045; C07D 491/107

(52) U.S. Cl. CPC C08G 75/045; C07D 491/107

(57) ABSTRACT

In various embodiments. the present invention relates to a recyclable, semi-crystalline, mechanochromic elastomeric composition for use in sensor, strain-sensing and shape-memory applications having mechanochromism, SH, and SIC properties that are each strain rate-dependent. In various embodiments, the present invention is directed to a dipropiolate-derivatized spiropyran (SP) mechanophore and thiol-yne-derived stereoelastomers doped with these SP mechanophores. These SP doped, thiol-yne-derived stereoelastomers may be synthesized via a base-directed Michael addition polymerization reaction. These linear polymers have been found to be semi-crystalline, recyclable, and mechanochromic under several methods of mechanical activation.

### CROSS REFERENCE TO RELATED APPLICATIONS

This application claims the benefit of U.S. provisional patent application Ser. No. 63/346,432 entitled “Mechanochromism and Strain-Induced Crystallization in Thiol-yne-Derived Stereoelastomers,” filed May 27, 2023, and incorporated herein by reference in its entirety.

### STATEMENT REGARDING FEDERALLY SPONSORED RESEARCH OR DEVELOPMENT SUPPORT

This invention was made with government support under Grant No. W911NF-20-2-0182 awarded by U.S. Army Research Office. The government has certain rights in the invention.

### FIELD OF THE INVENTION

One or more embodiments of the present invention relates to mechanochromophores for macroscopic visualization of molecular stresses. In certain embodiments, the present invention relates to thiol-yne-derived stereoelastomers, doped covalently with a dipropiolate-derivatized spiropyran (SP) mechanophore.

### BACKGROUND OF THE INVENTION

Semi-crystalline polymers are used widely in biomaterial and barrier film applications. These polymers contain distinct regions of crystalline and amorphous morphologies, which collectively impart often desirable properties, including elastomeric mechanical behavior. Under uniaxial tension in particular, semi-crystalline polymers often exhibit nonlinear stress responses that arise from the disassembly and formation of new crystalline regions. For example, at large deformations, the alignment of highly stretched polymer chains can lead to strain-induced crystallization (SIC). (See, e.g., J. Zhou, S. A. Turner, S. M. Brosnan, Q. Li, J. Y. Carrillo, D. Nykypanchuk, O. Gang, V. S. Ashby, A. V. Dobrynin, S. S. Sheiko, Macromolecules 2014, 47, 1768, the disclosure of which is incorporated by reference in its entirety. SIC results in significant material toughening due to interchain van der Waals interactions, which may be desirable or undesirable, depending on the application. Indeed, many polymer materials are processed using methods that specifically take advantage of SIC-based toughening. Therefore, SIC is critical to many industrial applications, including biomedical materials, pharmaceuticals, anti-tamper technology, and polymer-reinforced infrastructure.

The many morphological responses of semi-crystalline polymers to uniaxial deformation are well documented. A subset of those processes relevant to our considerations of SIC in this work is illustrated in FIG. 1A; a more complete schematic may be found in a recent review by Zhang et al. (See, Y. Zhang, P. Y. Ben Jar, S. Xue, L. Li, Journal of Materials Science 2018, 54, 62, the disclosure of which is incorporated herein by reference in its entirety. Initial polymer elasticity is attributed mainly to the amorphous, rubbery regions that are found between crystalline lamellar stacks. Extension of the amorphous chains (interlamellar shear) imposes stresses between the crystallites until they eventually fragment, marking the yield point. As strain increases further, the polymer deforms plastically, during which tensile stress remains mostly constant with increasing strain; the dominant processes during plastic deformation include the disentangling of amorphous chains and the shearing and melting of crystallites, among others. The uncoiling of these chains gradually reduces their conformational degrees of freedom as the polymer sub-chains approach their nominal contour lengths. Eventually, this gradual transition is accompanied by strain hardening (SH). At the molecular level, SH marks the transition from a reduction in conformational entropy, to an increase in the enthalpic distortion of chemical bonds. At the polymer chain level, this corresponds to large tensile forces within the individual chains. Macroscopically, this process is manifested in a nonlinear SH stress response, in which polymer stress increases rapidly with further strain. As the amorphous polymer chains are deformed further, they become more rigidly aligned, and the precise orientation of the chains enables SIC. SIC, a sub-type of SH, results in an anisotropic phase change in the newly-crystalline chains. Any subsequent polymer relaxation, both before and after failure, occurs primarily within the remaining amorphous regions.

Thiol-yne-derived stereoelastomers are one class of materials that undergo SIC under uniaxial tension. These elastomers are synthesized using a base-directed Michael addition step-growth polymerization (FIG. 1B). The relative stereochemistry of the resulting alkene backbone is determined by the polarity of the solvent, which influences the coordination of the base catalyst (and thus the equilibrium constant, K_{eq}) with the activated alkyne. Importantly, this tunable stereochemistry enables concomitant control over mechanical and thermal properties. (See, e.g., C. A. Bell, J. Yu, I. A. Barker, V. X. Truong, Z. Cao, A. V. Dobrynin, M. L. Becker, A. P. Dove, Angew. Chem. Int. Ed. 2016, 55, 13076; J. C. Worch, A. C. Weems, J. Yu, M. C. Arno, T. R. Wilks, R. T. R. Huckstepp, R. K. O'Reilly, M. L. Becker, A. P. Dove, Nat Commun 2020, 11, 3250; and M. B. Wandel, C. A. Bell, J. Yu, M. C. Arno, N. Z. Dreger, Y. H. Hsu, A. Pitto-Barry, J. C. Worch, A. P. Dove, M. L. Becker, Nat Commun 2021, 12, 446, the disclosures of which are incorporated herein by reference in their entirety). More specifically, an increase in cis content correlates with crystallinity due to enhanced crystal packing, and this correlation is observed similarly in proclivity towards SIC. (See, e.g., C. A. Bell, J. Yu, I. A. Barker, V. X. Truong, Z. Cao, A. V. Dobrynin, M. L. Becker, A. P. Dove, Angew. Chem. Int. Ed. 2016, 55, 13076, the disclosure of which is incorporated herein by reference in its entirety). These elastomers are linear, and although they are not cross-linked chemically, the crystalline domains serve as net-points that are linked weakly through van der Waals forces. As a result, when elastomers are stretched, tensile response is influenced highly by the relaxation kinetics associated with the reorganization of the crystalline domains. Macroscopically, this results in multi-regime SH and SIC behaviors that are strain rate-dependent in both onset and extent. Consequently, connections between these strain rate-dependent macroscopic properties with stress/strain dependent molecular behaviors have the potential to inform end use applications.

Mechanochromophores are stress-responsive molecules that, when incorporated within materials, provide macroscopic visualization of molecular stresses. These molecules undergo force-coupled switching between isomers that possess different spectroscopic properties. In general, mechanophores possess (comparatively) reactive sites, which are employed strategically so they will break, or otherwise react, prior to other irreversible scission events within the material. The activation of the mechanophore occurs as the polymer chains reach SH, when covalent bonds are under increased enthalpic distortions. For example, the widely-used spiropyran (SP) mechanophore is initially colorless because of its spirocyclic structure. With applied force, SP undergoes an electrocyclic ring-opening to merocyanine (MC), and this isomerization results in a visibly observable blue or purple color due to the increased co-planarity and conjugation of aromatic groups, as illustrated in FIG. 1C. The force-coupled equilibrium between these “off” (SP) and “on” (MC) states can then be monitored using absorbance or fluorescence spectroscopy. (See, e.g., D. A. Davis, A. Hamilton, J. Yang, L. D. Cremar, D. Van Gough, S. L. Potisek, M. T. Ong, P. V. Braun, T. J. Martinez, S. R. White, J. S. Moore, N. R. Sottos, Nature 2009, 459, 68; G. R. Gossweiler, G. B. Hewage, G. Soriano, Q. Wang, G. W. Welshofer, X. Zhao, S. L. Craig, ACS Macro Letters 2014, 3, 216; and Y. Chen, C. J. Yeh, Y. Qi, R. Long, C. Creton, Sci. Adv. 2020, 6, eaaz5093, the disclosures of which are incorporated herein by reference in their entirety). As a result, mechanophores facilitate macroscopic monitoring of molecular-level stresses within polymer materials.

The direct application of mechanophores to detecting and monitoring changes in bulk materials remains a burgeoning research area. Mechanophores have been used to probe specific processes including: shape-memory effects, visualization of polymer necking, temperature-induced crystallite formation, and quantitative stress-mapping. In these reports, mechanochromic responses to uniaxial deformation typically display the same behavior, in which activation is minimal through the yield point, increases close to linearly with further strain through SH, and, in some cases, plateaus just before material failure. (See, e.g., Y. Chen, C. J. Yeh, Y. Qi, R. Long, C. Creton, Sci. Adv. 2020, 6, eaaz5093 and J. W. Kim, Y. Jung, G. W. Coates, M. N. Silberstein, Macromolecules 2015, 48, 1335, the disclosures of which are incorporated herein by reference in their entirety). In most of these examples, mechanophore activation is independent of the applied strain rate, so there is minimal influence from viscoelastic relaxation. For the publications that do report strain rate-dependence, mechanophore activation is delayed by an additional 10-50% elongation per 100-fold decrease in rate. (See, e.g., J. W. Kim, Y. Jung, G. W. Coates, M. N. Silberstein, Macromolecules 2015, 48, 1335; C. M. Kingsbury, P. A. May, D. A. Davis, S. R. White, J. S. Moore, N. R. Sottos, J. Mater. Chem. 2011, 21, 8381; L. S. Shannahan, Y. Lin, J. F. Berry, M. H. Barbee, M. Fermen-Coker, S. L. Craig, Macromol. Rapid. Commun. 2021, 42, e2000449; and Y. Vidaysky, S. J. Yang, B. A. Abel, I. Agami, C. E. Diesendruck, G. W. Coates, M. N. Silberstein, J. Am. Chem. Soc. 2019, 141, 10060, the disclosures of which are incorporated herein by reference in their entirety). As such, the interplay of mechanochromism and strain rate-dependent SIC, particularly within thiol-yne stereoelastomers, is of interest.

Accordingly, what is needed in the art is a recyclable, semi-crystalline, mechanochromic elastomeric composition for use in sensor, strain-sensing and shape-memory applications having mechanochromism, SH, and SIC properties that are each strain rate-dependent.

### SUMMARY OF THE INVENTION

As set forth above, most elastomers undergo strain-induced crystallization (SIC) under tension; as individual chains are held rigidly in a fixed position by an applied strain, their alignment along the strain field results in a shift from strain-hardening (SH) to SIC. A similar degree of stretching is associated with covalent mechanochemical responses, raising the possibility of an interplay between the macroscopic response of SIC, and the molecular response of mechanophore activation. In various embodiments, the present invention relates to a recyclable, semi-crystalline, mechanochromic elastomeric composition for use in sensor, strain-sensing and shape-memory applications having mechanochromism, SH, and SIC properties that are each strain rate-dependent. In one or more of these embodiments, the present invention is directed to thiol-yne-derived stereoelastomers, doped covalently with a dipropiolate-derivatized spiropyran (SP) mechanophore. These semi-crystalline, mechanochromic stereoelastomers possessing SP mechanophores may be synthesized via well understood thiol-yne Michael addition polymerization reactions.

The material properties of these thiol-yne-derived stereoelastomers doped covalently with the dipropiolate-derivatized SP mechanophores are consistent with undoped controls, indicating that the SP behaves as a reporter of the mechanical state of the nascent polymer. Uniaxial tensile tests of these elastomers reveal correlations between mechanochromism, SH, and SIC, which are each strain rate-dependent. When these mechanochromic films are stretched slowly to the point of mechanophore activation, the covalently-tethered mechanophore remains trapped in a force-activated state, even after the applied stress is removed; additionally, the kinetics of mechanophore reversion correlate highly with the extent of SIC. Because these elastomers are not cross-linked, they are recyclable by melt-pressing into new films, which increases their potential range of strain-sensing and shape-memory applications.

In a first aspect, the present invention is directed to a dipropiolate-derivatized spiropyran (SP) mechanophore comprising two propriolate functional groups each joined by a C₂-C₁₀ alkyl chain and an ester linkage to a spiropyran molecule. In some embodiments, the C₂-C₁₀ alkyl chains are C₅H₁₀ alkyl chains. In one or more embodiment, the mechanophore exhibits a blue color when under strain.

In one or more embodiments, the dipropiolate-derivatized spiropyran (SP) mechanophore of the present invention includes any one or more of the above referenced embodiments of the first aspect of the present invention having the formula:

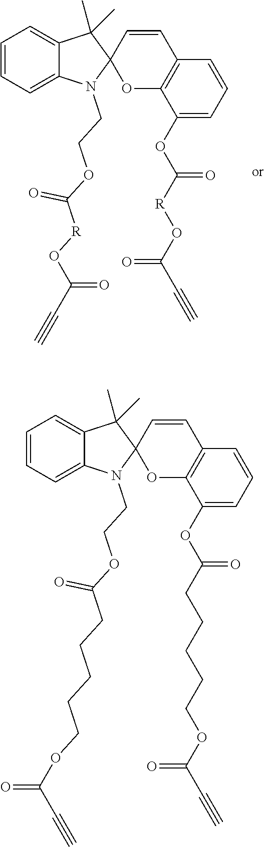

where R is a C₂-C₁₀ alkyl chain.

In a second aspect, the present invention is directed to a linear thiol-yne-derived stereoelastomer comprising the dipropiolate-derivatized spiropyran (SP) mechanophore described above wherein the linear thiol-yne-derived stereoelastomer elastomer exhibits strain induced mechanochromatism. In one or more embodiments, the linear thiol-yne-derived stereoelastomer comprises a polymer chain containing a plurality of stereoactive alkene bonds and 75% or more of these alkene bonds are in a cis orientation.

In one or more embodiments, the thiol-yne-derived stereoelastomer of the present invention includes any one or more of the above referenced embodiments of the second aspect of the present invention wherein the stereoelastomer comprises a plurality of stereoactive alkene bonds and 80% or more of these alkene bonds are in a cis orientation. In one or more embodiments, the thiol-yne-derived stereoelastomer of the present invention includes any one or more of the above referenced embodiments of the second aspect of the present invention comprising the residues of two or more a C₂-C₂₀ dithiols and two or more C₂-C₈ dipropiolates. In one or more embodiments, the thiol-yne-derived stereoelastomer of the present invention includes any one or more of the above referenced embodiments of the second aspect of the present invention wherein the ratio of the two or more C₂-C₂₀ dithiols to the two or more C₂-C₈ dipropiolates is about 1:1. In one or more embodiments, the thiol-yne-derived stereoelastomer of the present invention includes any one or more of the above referenced embodiments of the second aspect of the present invention further comprising the residues of two or more 1,6-hexanedithiol and two or more propane-1,3-diyl dipropiolate monomers. In one or more embodiments, the thiol-yne-derived stereoelastomer of the present invention includes any one or more of the above referenced embodiments of the second aspect of the present invention wherein the ratio of the two or more 1,6-hexanedithiol monomers to the two or more propane-1,3-diyl dipropiolate monomers is about 1:1.

In one or more embodiments, the thiol-yne-derived stereoelastomer of the present invention includes any one or more of the above referenced embodiments of the second aspect of the present invention wherein the elastomer exhibits a visible blue color upon strain hardening. In one or more embodiments, the thiol-yne-derived stereoelastomer of the present invention includes any one or more of the above referenced embodiments of the second aspect of the present invention having no chemical crosslinks.

In one or more embodiments, the thiol-yne-derived stereoelastomer of the present invention includes any one or more of the above referenced embodiments of the second aspect of the present invention comprising the reaction product of the dipropiolate-derivatized spiropyran (SP) mechanophore of described above, a C₂-C₂₀ dithiol, and a C₂-C₈ dipropiolate.

In one or more embodiments, the thiol-yne-derived stereoelastomer of the present invention includes any one or more of the above referenced embodiments of the second aspect of the present invention comprising the reaction product of the dipropiolate-derivatized spiropyran (SP) mechanophore described above, 1,6-hexanedithiol, and propane-1,3-diyl dipropiolate.

In one or more embodiments, the thiol-yne-derived stereoelastomer of the present invention includes any one or more of the above referenced embodiments of the second aspect of the present invention wherein the elastomer comprises from about 0.001 wt. % to about 5 wt. %, preferably from about 0.01 wt. % to about 2.0 wt. %, and more preferably from about 0.8 wt. % to about 1.6 wt. % of the dipropiolate-derivatized SP mechanophores.

In one or more embodiments, the thiol-yne-derived stereoelastomer of the present invention includes any one or more of the above referenced embodiments of the second aspect of the present invention wherein the elastomer comprises about 1.0 wt. % of the dipropiolate-derivatized SP mechanophores. In one or more embodiments, the thiol-yne-derived stereoelastomer of the present invention includes any one or more of the above referenced embodiments of the second aspect of the present invention wherein the elastomer comprises about 1.5 wt. % of the dipropiolate-derivatized SP mechanophores. In one or more embodiments, the thiol-yne-derived stereoelastomer of the present invention includes any one or more of the above referenced embodiments of the second aspect of the present invention wherein the elastomer has substantially the same mechanical and thermal properties as comparable elastomers not comprising the dipropiolate-derivatized SP mechanophores.

In one or more embodiments, the thiol-yne-derived stereoelastomer of the present invention includes any one or more of the above referenced embodiments of the second aspect of the present invention having a Young's modulus of from about 70 MPa to about 150 MPa, preferably from about 80 MPa to about 130 MPa.

In some embodiments, the thiol-yne-derived stereoelastomer of the present invention includes any one or more of the above referenced embodiments of the second aspect of the present invention having the same mechanophoric properties after melt recycling.

In various embodiments, the thiol-yne-derived stereoelastomer of the present invention includes any one or more of the above referenced embodiments of the second aspect of the present invention having the formula:

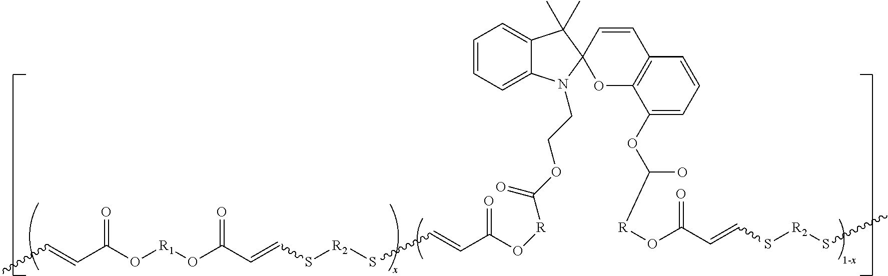

where x is a mole fraction from about 0.95 to about 0.999, preferably from about 0.980 to about 0.999, and more preferably from about 0.992 to about 0.996; R is a C₂-C₁₀ alkyl group; R₁ is a C₂ to C₈ alkyl group; R₂ is a C₂-C₂₀ alkyl group, an aryl group, an ether group, or a poly(ethylene glycol); and [symbol] indicates a stereoactive bond. Here, as well as elsewhere in the specification and claims, individual range values can be combined to form additional non-disclosed ranges. In some of these embodiments, x is a mole fraction of about 0.995, R is a C₅ alkyl group; R₁ is a C₃ alkyl group and R₂ is a C₆ alkyl group.

In various embodiments, the thiol-yne-derived stereoelastomer of the present invention includes any one or more of the above referenced embodiments of the second aspect of the present invention having the formula:

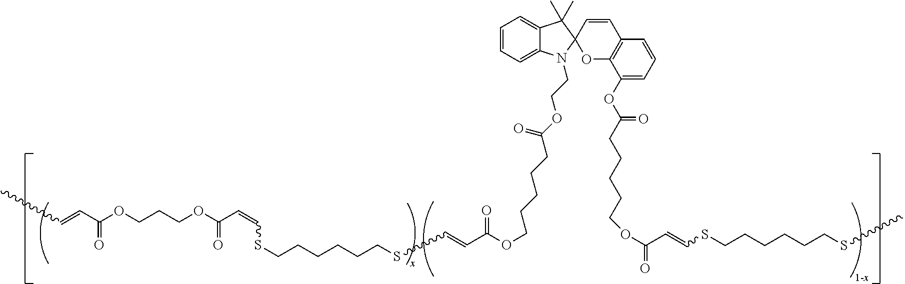

where x is a mole fraction of from about 0.950 to about 0.999. In one or more embodiments, the thiol-yne-derived stereoelastomer of the present invention includes any one or more of the above referenced embodiments of the second aspect of the present invention wherein x is a mole fraction of from about 0.950 to about 0.999, preferably from about 0.980 to about 0.999, and more preferably from about 0.992 to about 0.996. In one or more embodiments, x is a mole fraction of about 0.995. Here, as well as elsewhere in the specification and claims, individual range values can be combined to form additional non-disclosed ranges.

In a third aspect, the present invention is directed to a film comprising the thiol-yne-derived stereoelastomer described above.

In a fourth aspect, the present invention is directed to a thiol-yne-derived stereoelastomer comprising the reaction product of the dipropiolate-derivatized spiropyran (SP) mechanophore as described above, a C₂-C₂₀ dithiol, and a C₂-C₈ dipropiolate. In one or more embodiments, the linear thiol-yne-derived stereoelastomer of the present invention includes any one or more of the above referenced embodiments of the fourth aspect of the present invention comprising the reaction product of the dipropiolate-derivatized spiropyran (SP) mechanophore described herein, 1,6-hexanedithiol, and propane-1,3-diyl dipropiolate. In one or more embodiments, the linear thiol-yne-derived stereoelastomer of the present invention includes any one or more of the above referenced embodiments of the fourth aspect of the present invention wherein the elastomer comprises from about 0.001 wt. % to about 5 wt. %, preferably from about 0.01 wt. % to about 2.0 wt. %, and more preferably from about 0.8 wt. % to about 1.6 wt. % of the dipropiolate-derivatized SP mechanophores.

In one or more embodiments, the linear thiol-yne-derived stereoelastomer of the present invention includes any one or more of the above referenced embodiments of the fourth aspect of the present invention wherein the elastomer comprises about 1.0 wt. % of the dipropiolate-derivatized SP mechanophores. In one or more embodiments, the linear thiol-yne-derived stereoelastomer of the present invention includes any one or more of the above referenced embodiments of the fourth aspect of the present invention wherein the elastomer comprises about 1.5 wt. % of the dipropiolate-derivatized SP mechanophores. In one or more embodiments, the linear thiol-yne-derived stereoelastomer of the present invention includes any one or more of the above referenced embodiments of the fourth aspect of the present invention wherein the elastomer has substantially the same mechanical and thermal properties as comparable elastomers not comprising the dipropiolate-derivatized SP mechanophores. In one or more embodiments, the linear thiol-yne-derived stereoelastomer of the present invention includes any one or more of the above referenced embodiments of the fourth aspect of the present invention having a Young's modulus of from about 70 MPa to about 150 MPa, preferably from 80 MPa to about 130 MPa.

In a fifth aspect, the present invention is directed to a method for making the dipropiolate-derivatized spiropyran (SP) mechanophore described above comprising: dissolving a spiropyran (SP) diol and an organic base in a suitable solvent; adding a quantity of 6-bromohexanoic anhydride to the solution and stirring for from about 6 to about 18 hours to produce 2-(8-((6-bromohexanoyl)oxy)-3′,3′-dimethylspiro [chromene-2,2′-indolin]-1′-yl) ethyl 6-bromohexanoate; placing the 2-(8-((6-bromohexanoyl)oxy)-3′,3′-dimethylspiro [chromene-2,2′-indolin]-1′-yl) ethyl 6-bromohexanoate in a suitable reaction vessel in a dark location and adding sodium propiolate; sealing the reaction vessel and heating it to a curing temperature until cured; collecting and purifying the reaction product to produce the 3′,3′-dimethyl-1′-(2-((6-(propioloyloxy)hexanoyl) oxy)ethyl) spiro[chromene-2,2′-indolin]-8-yl 6-(propioloyloxy)hexanoate.

In some of these embodiments, the spiropyran (SP) diol has the formula:

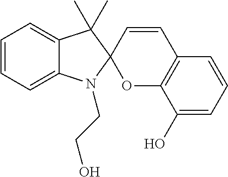 [SMILES: CC1(C)c2ccccc2N(CCO)C12C=Cc1cccc(O)c1O2]

In some embodiments, the organic base is N,N′-dimethylamino pyridine (DMAP). In one or more embodiments, the suitable solvent for the spiropyran (SP) diol and organic base is dichloromethane.

In one or more embodiments, the method for making the dipropiolate-derivatized spiropyran (SP) mechanophore of the present invention includes any one or more of the above referenced embodiments of the fifth aspect of the present invention wherein the step of adding further comprises: concentrating and then redissolving the 2-(8-((6-bromohexanoyl)oxy)-3′,3′-dimethylspiro [chromene-2,2′-indolin]-1′-yl) ethyl 6-bromohexanoate in ethyl acetate; washing the solution with water, sodium bicarbonate, and brine and then drying it on sodium sulfate; and concentrating the product onto neutral aluminum oxide and then purifying it via column chromatography.

In one or more embodiments, the method for making the dipropiolate-derivatized spiropyran (SP) mechanophore of the present invention includes any one or more of the above referenced embodiments of the fifth aspect of the present invention wherein the step of collecting and purifying comprises: transferring the reaction product into saturated NH₄Cl (200 mL) and stirring; extracting the crude product with ethyl acetate and then washing it in water and brine and drying it over Na₂SO₄; filtering and concentrated the crude product of onto silica gel, and then drying it under vacuum; and purifying the product by flash chromatography.

In another aspect, the present invention is directed to a method for making the thiol-yne-derived stereoelastomer described above comprising: combining dipropiolate-derivatized spiropyran (SP) mechanophore as described above, a C₂-C₂₀ dithiol, and a first quantity of a C₂-C₈ dipropiolate in a suitable reaction vessel; cooling the reaction vessel to a temperature of from about −20° C. to about −5° C. and stirring for from about 14 min to about 60 min; separately combining 1,8-diazabicyclo[5.4.0]undec-7-ene (DBU) and a polar solvent or solvent combination and slowly adding the combination to the reaction vessel; allowing the reaction vessel to warm to room temperature and after from about 15 to about 45 min, dissolving a second quantity of the C₂-C₈ dipropiolate in a suitable solvent; adding the C₂-C₈ dipropiolate solution to the reaction vessel to end-cap any unreacted thiol groups; adding a radical scavenger to the reaction vessel to prevent crosslinking; and collecting a purifying the reaction product to produce the thiol-yne-derived stereoelastomer described above. In some of these embodiments, the C₂-C₂₀ dithiol is 1,6-hexanedithiol. In some of these embodiments, the C₂-C₈ dipropiolate is propane-1,3-diyl dipropiolate. In some of these embodiments, the step of slowly adding comprises adding to the DBU solution dropwise over a period of about 20 min. In various embodiments, the radical scavenger is butylated hydroxytoluene (BHT). In some embodiments, the step of collecting a purifying further comprises: precipitating the product into Et₂O; collecting the thiol-yne-derived stereoelastomer by decanting the supernatant; drying the product under vacuum for from about 12 to about 36 hours to produce the purified polymer thiol-yne-derived stereoelastomer described above.

### BRIEF DESCRIPTION OF THE DRAWINGS

For a more complete understanding of the features and advantages of the present invention, reference is now made to the detailed description of the invention along with the accompanying figures in which:

FIGS. 1A-D are a series of schematic diagrams and reaction schemes showing mechanochromism in semi-crystalline polymers including: schematic of selected aspects of structural evolution in semi-crystalline, mechanochromic polymers under tension (adapted from Zhang et al., where a more complete description of the full range of responses can be found (See. Y. Zhang, P. Y. Ben Jar, S. Xue, L. Li, Journal of Materials Science 2018, 54, 62, the disclosure of which is incorporated herein by reference in its entirety)) (FIG. 1A); synthesis of thiol-yne stereoelastomers (FIG. 1B); force-coupled equilibrium of SP and MC (FIG. 1C); and a schematic illustration of polymers in relaxed SP state (top), strain-hardened MC (middle) and force-trapped MC due to SIC (bottom) (FIG. 1D).

FIGS. 2A-D are a series of SEC chromatograms for all (0-SP (FIG. 2A), 1-SP (FIG. 2B), 1.5 SP (FIG. 2C), and 1-SP_{recycled} (FIG. 2D)) polymers. Molecular weights given are the weight average molecular weights (M_{w}).

FIGS. 3A-C are images showing example methods of SP activation in 1-SP before and after cutting bulk, unprocessed 1-SP (FIGS. 3A-B) and stretching melt-pressed 1-SP (FIG. 3C).

FIGS. 4A-L is a series of example stress/strain curves for 0-SP (FIGS. 4A-C), 1-SP (FIGS. 4D-F), 1.5 SP (FIGS. 4G-I), and 1-SP recycled recycled (FIGS. 4J-L)polymers. Please note that the 10 mm/min-tested 0-SP (FIG. 4A and 1-SP (FIG. 4D) samples slipped moderately around 500% strain, resulting in dips in tensile stress.

FIGS. 5A-D are a series of graphs showing examination of the effect of \dot{ε} and SP content on mechanical properties. All parts include triplicate data for 0-SP, 1-SP, and 1.5-SP, across all \dot{ε} (i.e., 27 individual tensile tests). Specifically, FIG. 5A shows stress vs. at early deformations; FIG. 5B shows [symbol] E(t) [symbol] vs. time; FIG. 5C shows stress vs. over full deformation range; and FIG. 5D shows σ_{True}(t)/\dot{ε} vs. time. In FIG. 5B and FIG. 5D, time, t=/\dot{ε}.

FIGS. 6A-E are a series of graphs showing mechanical property dependence on SP content including: average yield point strain (%) (FIG. 6A), average yield point stress (MPa) (FIG. 6B), average elongation at break (%) (FIG. 6C), average stress at break (MPa) (FIG. 6D), and average elastic modulus (MPa) (FIG. 6E). All data are reported as average ±1 standard deviation of n=3 samples. All stress and strain are engineering values.

FIGS. 7A-B is a series of graphs showing true stress vs. true elongation (labeled as strain) for all tensile tests at linear (FIG. 7A) and log (FIG. 7B) scales. (σ_{True}=σ(1+λ); λ_{True}=ln (1+λ)).

FIGS. 8A-H are a series of DSC thermograms for all stereoelastomers (0-SP (FIGS. 8A-B), 1-SP (FIGS. 8C-D), 1.5 SP (FIGS. 8E-F), and 1 -SP_{recycled} (FIGS. 8G-H)).

FIG. 9 shows DSC thermograms demonstrating \dot{ε}-dependent SIC in 0-SP for unstretched (top), stretched at \dot{ε}₁₀ (middle) and stretched at \dot{ε}₁ (bottom) samples.

FIGS. 10A-F are two rounds of DSC traces for 0-SP unstretched (FIGS. 10A, 10D), stretched at 10 mm/min (FIGS. 10B, 10E), and stretched at 1 mm/min (FIGS. 10C, 10F) demonstrating strain rate-dependent SIC.

FIGS. 11A-C are a series of graphs showing examples of pixel data calculations from 1-SP tested at 10 mm/min (FIG. 11A). The raw mean pixel intensities obtained from Fiji analysis. This data was used to calculate the chromaticity of each color channel, as shown in (FIG. 11B). All data was then normalized to their initial values, giving each channel's chromatic change, shown in FIG. 11C.

FIGS. 12A-C are a series of graphs showing example RGB chromatic change curves for 0-SP (FIG. 12A), 1-SP (FIG. 12B), and 1.5-SP (FIG. 12C).

FIGS. 13A-B are a series of example images (FIG. 13A) and ΔS_{blue} curves (FIG. 13B) for 0-SP, 1-SP, and 1.5-SP, all for \dot{ε}₁₀.

FIGS. 14A-F are a series of graphs showing strain rate-dependent mechanochromism in 1-SP. Images captured between=100-800% while stretching at \dot{ε}₁₀ (FIG. 14A), \dot{ε}₁ (FIG. 14B), and \dot{ε}_{0.1}. (FIG. 14C). The corresponding ΔS_{blue} and tensile curves are pictured in FIG. 14D, (FIG. 14E), and (FIG. 14F), respectively.

FIGS. 15A-C are a series of graphs showing tensile and pixel data overlayed for 0-SP polymers at 10 mm/min strain rate. Please note that 2 samples (FIG. 15B and FIG. 15C) slipped moderately around 400% strain.

FIGS. 16A-I are a series of graphs showing tensile and pixel data overlayed for 1-SP. The graphs represent triplicate samples tested at 10 mm/min (FIGS. 16A-C), 1 mm/min (FIGS. 16D-F), and 0.1 mm/min (FIG. 16G-I).

FIGS. 17A-I are a series of graphs showing tensile and pixel data overlayed for 1.5-SP. The graphs represent triplicate samples tested at 10 mm/min (FIGS. 17A-C), 1 mm/min (FIGS. 17D-F), and 0.1 mm/min (FIGS. 17G-I).

FIGS. 18A-I are a series of graphs showing tensile and pixel data overlayed for 1-SP_{Reset}. The graphs represent triplicate samples tested at 10 mm/min (FIGS. 18A-C), 1 mm/min (FIGS. 18D-F), and 0.1 mm/min (FIGS. 18G-I).

FIGS. 19A-B are a ΔS_{blue} curve (FIG. 19A) and example images (FIG. 19B) showing polymer whitening (PW) effects and multi-regime mechanochromism in 0-SP.

FIGS. 20A-B are a ΔS_{blue} curve (FIG. 20) and example images (FIG. 20B) showing polymer whitening (PW) effects and multi-regime mechanochromism in 1-SP.

FIGS. 21A-I are a series of graphs showing curve fits with linear regressions for SP off regime (FIGS. 21A, D, G) and SP on regime showing λ_{Act} for 1-SP at a speed of 10 mm/min (FIGS. 21B, E, H). The intersection of these regressions is the extrapolated strain at activation, λ_{Act} (labeled SP-ON). λ_{Act} and linear regressions are displayed with chromatic change data over the full tensile range. (FIGS. 21C, E, I). Each Figure contains results for triplicate samples.

FIGS. 22A-I are a series of graphs showing curve fits with linear regressions for SP off regime (FIGS. 22A, D, G) and SP on regime showing λ_{Act} for 1-SP at a speed of 1 mm/min (FIGS. 22B, E, H). The intersection of these regressions is the extrapolated strain at activation, λ_{Act} (labeled SP-ON λ_{Act}) and linear regressions are displayed with chromatic change data over the full tensile range. (FIGS. 22C, E, I). Each Figure contains results for triplicate samples.

FIGS. 23A-I are a series of graphs showing curve fits with linear regressions for SP off regime and (FIGS. 23A, D, G) SP on regime showing λ_{Act} for 1-SP at a speed of 0.1 mm/min (FIGS. 23B, E, H). The intersection of these regressions is the extrapolated strain at activation, λ_{Act} (labeled SP-ON). λ_{Act} and linear regressions are displayed with chromatic change data over the full tensile range (FIGS. 23C, E, I). Each Figure contains results for triplicate samples.

FIGS. 24A-I are a series of graphs showing curve fits with linear regressions for SP off regime (FIGS. 24A, D, G) and SP on regime showing λ_{Act} for 1.5-SP at a speed of 10 mm/min (FIGS. 24B, E, H). The intersection of these regressions is the extrapolated strain at activation, λ_{Act} (labeled SP-ON). λ_{Act} and linear regressions are displayed with chromatic change data over the full tensile range (FIGS. 24C, E, I). Each Figure contains results for triplicate samples.

FIGS. 25A-I are a series of graphs showing curve fits with linear regressions for SP off regime (FIGS. 25A, D, G) and SP on regime showing λ_{Act} for 1.5-SP at a speed of 1 mm/min (FIGS. 25B, E, H). The intersection of these regressions is the extrapolated strain at activation, λ_{Act} (labeled SP-ON). λ_{Act} and linear regressions are displayed with chromatic change data over the full tensile range (FIGS. 25C, E, I). Each Figure contains results for triplicate samples.

FIGS. 26A-I are a series of graphs showing curve fits with linear regressions for SP off regime (FIGS. 26A, D, G) and SP on regime showing λ_{Act} for 1.5-SP at a speed of 0.1 mm/min (FIGS. 26B, E, H). The intersection of these regressions is the extrapolated strain at activation, λ_{Act} (labeled SP-ON). λ_{Act} and linear regressions are displayed with chromatic change data over the full tensile range (FIGS. 26C, E, I). Each Figure contains results for triplicate samples.

FIGS. 27A-I are a series of graphs showing curve fits with linear regressions for SP off regime (FIGS. 27A, D, G) and SP on regime showing λ_{Act} for 1-SP_{Reset} at a speed of 10 mm/min (FIGS. 27B, E, H). The intersection of these regressions is the extrapolated strain at activation, λ_{Act} (labeled SP-ON). λ_{Act} and linear regressions are displayed with chromatic change data over the full tensile range. Each Figure contains results for triplicate samples.

FIGS. 28A-I are a series of graphs showing curve fits with linear regressions for SP off regime (FIGS. 28A, D, G) and SP on regime showing λ_{Act} for 1-SP_{Reset} at a speed of 1 mm/min (FIGS. 28B, E, H). The intersection of these regressions is the extrapolated strain at activation, λ_{Act} (labeled SP-ON). λ_{Act} and linear regressions are displayed with chromatic change data over the full tensile range(FIGS. 28C, E, I). Each Figure contains results for triplicate samples.

FIG. 29A-I are a series of graphs showing curve fits with linear regressions for SP off regime (FIGS. 29A, D, G) and SP on regime showing λ_{Act} for 1-SP_{Reset} at a speed of 0.1 mm/min (FIGS. 29B, E, H). The intersection of these regressions is the extrapolated strain at activation, λ_{Act} (labeled SP-ON). λ_{Act} and linear regressions are displayed with chromatic change data over the full tensile range(FIGS. 29C, E, I). Each Figure contains results for triplicate samples.

FIGS. 30A-F are images and graphs showing SP recovery kinetics for 1-SP, 10 mm/min stain rate, where FIG. 30A is a screenshot of 4 different ROIs used within the sample; the kinetics data associated with these ROIs are plotted in FIG. 30B. Each individual ROI 1-4 was fit with a 1-phase exponential decay model according to Equation (7), and these fittings are shown in FIGS. 30C-F, respectively. The average rate constant is 0.301±0.078 min⁻¹ (±1 standard deviation, n=4).

FIGS. 31A-F are images and graphs showing SP recovery kinetics for 1-SP, 10 mm/min strain rate, where FIG. 21A is a screenshot of 4 different ROIs used within the sample; the kinetics data associated with these ROIs are plotted in FIG. 31B. Each individual ROI 1-4 was fit with a 1-phase exponential decay model according to Equation (7), and these fittings are shown in FIGS. 31C-F, respectively. The average rate constant is 0.369±0.063 min⁻¹ (±1 standard deviation, n=4).

FIGS. 32A-F are images and graphs showing SP recovery kinetics for 1-SP, 10 mm/min strain rate, where FIG. 32A is a screenshot of 4 different ROIs used within the sample; the kinetics data associated with these ROIs are plotted in FIG. 32B. Each individual ROI 1-4 was fit with a 1-phase exponential decay model according to Equation (7), and these fittings are shown in FIGS. 32C-F, respectively. The average rate constant is 0.448±0.075 min⁻¹ (±1 standard deviation, n=4).

FIGS. 33A-F are images and graphs showing SP recovery kinetics for 1-SP, 1 mm/min strain rate, where FIG. 33A is a screenshot of 4 different ROIs used within the sample; the kinetics data associated with these ROIs are plotted in FIG. 33B. Each individual ROI 1-4 was fit with a 1-phase exponential decay model according to Equation (7), and these fittings are shown in FIGS. 33C-F, respectively. The average rate constant is 0.248±0.065 min−1 (±1 standard deviation, n=4).

FIGS. 34A-F are images and graphs showing SP recovery kinetics for 1-SP, 1 mm/min strain rate, where FIG. 34A is a screenshot of 4 different ROIs used within the sample; the kinetics data associated with these ROIs are plotted in FIG. 34B. Each individual ROI 1-4 was fit with a 1-phase exponential decay model according to Equation (7), and these fittings are shown in FIGS. 34C-F, respectively. The average rate constant is 0.236±0.041 min⁻¹ (±1 standard deviation, n=4).

FIGS. 35A-F are images and graphs showing SP recovery kinetics for 1-SP, 1 mm/min strain rate, where FIG. 35A is a screenshot of 4 different ROIs used within the sample; the kinetics data associated with these ROIs are plotted in FIG. 35B. Each individual ROI 1-4 was fit with a 1-phase exponential decay model according to Equation (7), and these fittings are shown in FIGS. 35C-F, respectively. The average rate constant is 0.264±0.051 min⁻¹ (±1 standard deviation, n=4).

FIGS. 36A-C are images showing \dot{ε}-dependent relaxation in 1-SP after stretching at \dot{ε}₁₀ (FIG. 36A), \dot{ε}₁ (FIG. 36B), and \dot{ε}_{0.1} (FIG. 36C).

FIGS. 37A-C are a graph (FIG. 37A) and images (FIGS. 37B-C) showing SP persistence in 1-SP_{Reset} (0.1 mm/min).

FIGS. 38A-F are images and graphs showing SP recovery kinetics for 1-SP, 0.1 mm/min strain rate, where FIG. 38A is a screenshot of 4 different ROIs used within the sample; the kinetics data associated with these ROIs are plotted in FIG. 38B. Each individual ROI 1-4 was fit with a 1-phase exponential decay model according to Equation (7), and these fittings are shown in FIGS. 38C-F, respectively. The average rate constant is 0.106±0.016 min⁻¹ (±1 standard deviation, n=4).

FIGS. 39A-F are images and graphs showing SP recovery kinetics for 1-SP, 0.1 mm/min strain rate, where FIG. 39A is a screenshot of 4 different ROIs used within the sample; the kinetics data associated with these ROIs are plotted in FIG. 39B. Each individual ROI 1-4 was fit with a 1-phase exponential decay model according to Equation (7), and these fittings are shown in FIGS. 39C-F, respectively. The average rate constant is 0.073±0.008 min⁻¹ (±1 standard deviation, n=4).

FIGS. 40A-F are images and graphs showing SP recovery kinetics for 1-SP, 0.1 mm/min strain rate, where FIG. 40A is a screenshot of 4 different ROIs used within the sample; the kinetics data associated with these ROIs are plotted in FIG. 40B. Each individual ROI 1-4 was fit with a 1-phase exponential decay model according to Equation (7), and these fittings are shown in FIGS. 40C-F, respectively. The average rate constant is 0.082±0.003 min⁻¹ (±1 standard deviation, n=4).

FIGS. 41A-F are images and graphs showing SP recovery kinetics for 1.5-SP, 10 mm/min strain rate, where FIG. 41A is a screenshot of 4 different ROIs used within the sample; the kinetics data associated with these ROIs are plotted in FIG. 41B. Each individual ROI 1-4 was fit with a 1-phase exponential decay model according to Equation (7), and these fittings are shown in FIGS. 41C-F, respectively. The average rate constant is 0.377±0.167 min⁻¹ (±1 standard deviation, n=4).

FIGS. 42A-F are images and graphs showing SP recovery kinetics for 1.5-SP, 10 mm/min strain rate, where FIG. 42A is a screenshot of 4 different ROIs used within the sample; the kinetics data associated with these ROIs are plotted in FIG. 42B. Each individual ROI 1-4 was fit with a 1-phase exponential decay model according to Equation (7), and these fittings are shown in FIGS. 42C-F, respectively. The average rate constant is 0.459±0.123 min⁻¹ (±1 standard deviation, n=4).

FIGS. 43A-F are images and graphs showing SP recovery kinetics for 1.5-SP, 10 mm/min strain rate, where FIG. 43A is a screenshot of 4 different ROIs used within the sample; the kinetics data associated with these ROIs are plotted in FIG. 43B. Each individual ROI 1-4 was fit with a 1-phase exponential decay model according to Equation (7), and these fittings are shown in FIGS. 43C-F, respectively. The average rate constant is 0.504±0.229 min⁻¹ (±1 standard deviation, n=4).

FIGS. 44A-F are images and graphs showing SP recovery kinetics for 1.5-SP, 1 mm/min strain rate, where FIG. 44A is a screenshot of 4 different ROIs used within the sample; the kinetics data associated with these ROIs are plotted in FIG. 44B. Each individual ROI 1-4 was fit with a 1-phase exponential decay model according to Equation (7), and these fittings are shown in FIGS. 44C-F, respectively. The average rate constant is 0.290±0.040 min⁻¹ (±1 standard deviation, n=4).

FIGS. 45A-F are images and graphs showing SP recovery kinetics for 1.5-SP, 1 mm/min strain rate, where FIG. 45A is a screenshot of 4 different ROIs used within the sample; the kinetics data associated with these ROIs are plotted in FIG. 45B. Each individual ROI 1-4 was fit with a 1-phase exponential decay model according to Equation (7), and these fittings are shown in FIGS. 45C-F, respectively. The average rate constant is 0.276±0.081 min⁻¹ (±1 standard deviation, n=4).

FIGS. 46A-F are images and graphs showing SP recovery kinetics for 1.5-SP, 1 mm/min strain rate, where FIG. 46A is a screenshot of 4 different ROIs used within the sample; the kinetics data associated with these ROIs are plotted in FIG. 46B. Each individual ROI 1-4 was fit with a 1-phase exponential decay model according to Equation (7), and these fittings are shown in FIGS. 46C-F, respectively. The average rate constant is 0.289±0.085 min⁻¹ (±1 standard deviation, n=4).

FIGS. 47A-F are images and graphs showing SP recovery kinetics for 1.5-SP, 0.1 mm/min strain rate, where FIG. 47A is a screenshot of 4 different ROIs used within the sample; the kinetics data associated with these ROIs are plotted in FIG. 47B. Each individual ROI 1-4 was fit with a 1-phase exponential decay model according to Equation (7), and these fittings are shown in FIGS. 47C-F, respectively. The average rate constant is 0.068±0.010 min⁻¹ (±1 standard deviation, n=4).

FIGS. 48A-F are images and graphs showing SP recovery kinetics for 1.5-SP, 0.1 mm/min strain rate, where FIG. 48A is a screenshot of 4 different ROIs used within the sample; the kinetics data associated with these ROIs are plotted in FIG. 48B. Each individual ROI 1-4 was fit with a 1-phase exponential decay model according to Equation (7), and these fittings are shown in FIGS. 48C-F, respectively. The average rate constant is 0.077±0.005 min⁻¹ (±1 standard deviation, n=4).

FIGS. 49A-F are images and graphs showing SP recovery kinetics for 1.5-SP, 0.1 mm/min strain rate, where FIG. 49A is a screenshot of 4 different ROIs used within the sample; the kinetics data associated with these ROIs are plotted in FIG. 49B. Each individual ROI 1-4 was fit with a 1-phase exponential decay model according to Equation (7), and these fittings are shown in FIGS. 49C-F, respectively. The average rate constant is 0.076±0.011 min⁻¹ (±1 standard deviation, n=4).

FIGS. 50A-F are images and graphs showing SP recovery kinetics for 1-SP_{Reset}, 10 mm/min strain rate, where FIG. 50A is a screenshot of 4 different ROIs used within the sample; the kinetics data associated with these ROIs are plotted in FIG. 50B. Each individual ROI 1-4 was fit with a 1-phase exponential decay model according to Equation (7), and these fittings are shown in FIGS. 50C-F, respectively. The average rate constant is 0.301±0.177 min⁻¹ (±1 standard deviation, n=4).

FIGS. 51A-F are images and graphs showing SP recovery kinetics for 1-SP_{Reset}, 10 mm/min strain rate, where FIG. 51A is a screenshot of 4 different ROIs used within the sample; the kinetics data associated with these ROIs are plotted in FIG. 51B. Each individual ROI 1-4 was fit with a 1-phase exponential decay model according to Equation (7), and these fittings are shown in FIGS. 51C-F, respectively. The average rate constant is 0.259±0.154 min⁻¹ (±1 standard deviation, n=4).

FIGS. 52A-F are a series of images and graphs showing SP recovery kinetics for 1-SP_{Reset}, 10 mm/min strain rate, where FIG. 52A is a screenshot of 4 different ROIs used within the sample; the kinetics data associated with these ROIs are plotted in FIG. 52B. Each individual ROI 1-4 was fit with a 1-phase exponential decay model according to Equation (7), and these fittings are shown in FIGS. 52C-F, respectively. The average rate constant is 0.282±0.064 min⁻¹ (±1 standard deviation, n=4).

FIGS. 53A-F are a series of images and graphs showing SP recovery kinetics for 1-SP_{Reset}, 1 mm/min strain rate, where FIG. 53A is a screenshot of 4 different ROIs used within the sample; the kinetics data associated with these ROIs are plotted in FIG. 53B. Each individual ROI 1-4 was fit with a 1-phase exponential decay model according to Equation (7), and these fittings are shown in FIGS. 53C-F, respectively. The average rate constant is 0.206±0.016 min⁻¹ (±1 standard deviation, n=4).

FIGS. 54A-F are a series of images and graphs showing SP recovery kinetics for 1-SP_{Reset}, 1 mm/min strain rate, where FIG. 54A is a screenshot of 4 different ROIs used within the sample; the kinetics data associated with these ROIs are plotted in FIG. 54B. Each individual ROI 1-4 was fit with a 1-phase exponential decay model according to Equation (7), and these fittings are shown in FIGS. 54C-F, respectively. The average rate constant is 0.217±0.039 min⁻¹ (±1 standard deviation, n=4).

FIGS. 55A-F are a series of images and graphs showing SP recovery kinetics for 1-SP_{Reset}, 1 mm/min strain rate, where FIG. 55A is a screenshot of 4 different ROIs used within the sample; the kinetics data associated with these ROIs are plotted in (b). Each individual ROI 1-4 was fit with a 1-phase exponential decay model according to Equation (7), and these fittings are shown in FIGS. 55C-F, respectively. The average rate constant is 0.228±0.023 min⁻¹ (±1 standard deviation, n=4).

FIGS. 56A-F are a series of images and graphs showing SP recovery kinetics for 1-SP_{Reset}, 0.1 mm/min strain rate, where FIG. 56A is a screenshot of 4 different ROIs used within the sample; the kinetics data associated with these ROIs are plotted in FIG. 56B. Each individual ROI 1-4 was fit with a 1-phase exponential decay model according to Equation (7), and these fittings are shown in FIGS. 56C-F, respectively. The average rate constant is 0.112±0.035 min⁻¹ (±1 standard deviation, n=4).

FIGS. 57A-F are a series of images and graphs showing SP recovery kinetics for 1-SP_{Reset}, 0.1 mm/min strain rate, where FIG. 57A is a screenshot of 4 different ROIs used within the sample; the kinetics data associated with these ROIs are plotted in FIG. 57B. Each individual ROI 1-4 was fit with a 1-phase exponential decay model according to Equation (7), and these fittings are shown in FIGS. 57C-F, respectively. The average rate constant is 0.099±0.006 min⁻¹ (±1 standard deviation, n=4).

FIGS. 58A-F are a series of images and graphs showing SP recovery kinetics for 1-SP_{Reset}, 0.1 mm/min strain rate, where FIG. 58A is a screenshot of 4 different ROIs used within the sample; the kinetics data associated with these ROIs are plotted in FIG. 58B. Each individual ROI 1-4 was fit with a 1-phase exponential decay model according to Equation (7), and these fittings are shown in FIGS. 58C-F, respectively. The average rate constant is 0.072±0.004 min⁻¹ (±1 standard deviation, n=4).

FIGS. 59A-D is a series of an image and graphs showing stress relaxation in 1-SP including a stress vs. curve, stretching to 600% (FIG. 59A), followed by stress relaxation while held at 600% (FIG. 59B); a graph of mechanochromism in 1-SP during and after stress relaxation (FIG. 59C), with associated images included in FIG. 59D.

FIGS. 60A-D is a series of graphs showing stress relaxation tensile data and relaxation kinetics after stretching to 600% λ at 10 m/min (FIGS. 60A-B) and 1 mm/min (FIGS. 60C-D).

FIGS. 61A-D is a series of graphs showing kinetics curve fits for stress relaxation kinetics tests after stretching to 600% λ at 10 m/min (FIGS. 61A-B) and 1 mm/min (FIGS. 61C-D).

FIGS. 62A-D is a series of graphs showing blue pixel data kinetics during stress relaxation tests and after releasing clamp. Samples were stretched at 10 m/min (FIGS. 62A-B) and 1 mm/min (FIGS. 62C-D) and held at 600% λ until relaxing to approximately 7-9 MPa, then bottom clamp was released.

FIGS. 63A-B are a series of graphs showing recycled 1-SP_{Reset} rate-dependent mechanochromism for average λ_{Act}, (±1 standard deviation, n=3) (FIG. 63A) and average decay constants (±1 standard deviation, n=12), plotted as functions of \dot{ε} (FIG. 63B). Data are plotted with 1-SP and 1.5-SP data for comparison, and p-values comparing 1-SP_{Reset} to 1-SP (2-tailed Student T-test) are included for each \dot{ε}.

FIG. 64 is a ¹H NMR spectra of 2-(8-((6-bromohexanoyl)oxy)-3′,3′-dimethylspiro [chromene-2,2′-indolin]-1′-yl) ethyl 6-bromohexanoate (molecule 3) (500 MHz, CDCl₃).

FIG. 65 is a ¹³C NMR spectra of 2-(8-((6-bromohexanoyl)oxy)-3′,3′-dimethylspiro [chromene-2,2′-indolin]-1′-yl) ethyl 6-bromohexanoate (molecule 3) (126 MHz, CDCl₃).

FIG. 66 is a ¹H NMR spectra of 3′,3′-dimethyl-1′-(2-((6-(propioloyloxy)hexanoyl)oxy)ethyl)spiro[chromene-2,2′-indolin]-8-yl 6-(propioloyloxy) hexanoate (molecule 4) (500 MHz, CDCl₃).

FIG. 67 is a ¹³C NMR spectra of 3′,3′-dimethyl-1′-(2-((6-(propioloyloxy)hexanoyl)oxy)ethyl)spiro[chromene-2,2′-indolin]-8-yl 6-(propioloyloxy) hexanoate (molecule 4) (126 MHz, CDCl₃).

FIG. 68 is a ¹H NMR spectra of polymer 0-SP (500 MHz, CDCl₃).

FIG. 69 is a ¹H NMR spectra of polymer 1-SP (500 MHz, CDCl₃).

FIG. 70 is a ¹H NMR spectra of polymer 1.5-SP (500 MHz, CDCl₃).

The patent or application file contains at least one drawing executed in color. Copies of this patent or patent application publication with color drawings will be provided by the Office upon request and payment of the necessary fee.

### DETAILED DESCRIPTION OF THE ILLUSTRATIVE EMBODIMENTS

The following is a detailed description of the disclosure provided to aid those skilled in the art in practicing the present disclosure. Those of ordinary skill in the art may make modifications and variations in the embodiments described herein without departing from the spirit or scope of the present disclosure. Unless otherwise defined, all technical and scientific terms used herein have the same meaning as commonly understood by one of ordinary skill in the art to which this disclosure belongs. The terminology used in the description of the disclosure herein is for describing particular embodiments only and is not intended to be limiting of the disclosure.

As set forth above, for several decades, elastomers have been investigated intensely because of their excellent performance in biomaterial, shape-memory, and barrier film applications. Most elastomers undergo strain-induced crystallization (SIC) while under tension. The degree of enthalpic distortion that results in SIC is also the basis for covalent mechanophore activation, which suggests a possible interplay between the macroscopic response of SIC and molecular response of mechanophore activation.

In response to this potential relationship, the present invention utilizes a dipropiolate-functionalized spiropyran (SP) mechanophore monomers, which we have incorporated covalently in thiol-yne-derived stereoelastomers with about 80% cis content along their backbone. These linear elastomers contain no chemical cross-links, so tensile response is influenced highly by chain entanglements and the breaking and forming of crystalline domains. While not wishing to be bound by theory, it is believed that testing these elastomers at different strain rates would facilitate comparison of mechanochromic responses to macroscopic material properties.

As set forth above, the present invention relates to new mechanochromic elastomers possessing unparalleled strain-rate-dependent mechanochromism, as well as the synthesis and characterization of a novel SP monomer and its incorporation in thiol-yne-derived stereoelastomers possessing about 80% cis content. Simply put, it is believed that the mechanochromism properties exhibited by these elastomers is unparalleled in the existing mechanochemistry portfolio and that this multidisciplinary work will be of particular interest to both polymer chemists/physicists, materials scientists, and engineers of both synthetics and/or computational backgrounds.

Creating materials with divergent mechanochromism properties usually requires significant microstructure changes, such as monomer composition, cross-linking density, or even the selected mechanophore. Typically, such changes also bias the mechanophore's force-coupled equilibrium, subsequently affecting the function and performance of the mechanochemical response, ultimately limiting their use in industrial applications. Here, however, the profound tunability of these materials is accomplished by varying the applied strain rate.

As set forth above, the present invention relates to a recyclable, semi-crystalline, mechanochromic elastomeric composition for use in sensor, strain-sensing and shape-memory applications having mechanochromism, SH, and SIC properties that are each strain rate-dependent. Tensile tests across three decades of strain rates reveal significant correlations between strain hardening (SH), SIC, and mechanochromism, which are each dependent on the applied strain rate. These new semi-crystalline thiol-yne-derived materials, are doped with covalently-tethered SP mechanophores. Elastomers with SP concentrations of 1 and 1.5 wt. % of the bulk were synthesized at multi-gram scales. Mechanical and thermal properties of these SP-doped elastomers are indistinguishable from undoped controls, confirming that the added SP behaves as a true sensor.

In addition, these elastomers display unprecedented strain rate-dependence in mechanophore activation. For a 10-fold decrease in strain rate, SP activation is delayed by approximately 100% additional tensile elongation. This significant delay is an order of magnitude larger than other reports of rate-dependent SP activation onset. Moreover, it has also been shown that these SP elastomers exhibit multi-regime mechanochromism, in which regimes of molecular mechanophore response correlate highly with the macroscopic regimes of SH and SIC. This indicates that mechanochemistry may successfully probe SIC in elastomeric materials.

In various embodiments, the present invention is directed to a dipropiolate-derivatized spiropyran (SP) mechanophore and thiol-yne-derived stereoelastomers doped with these SP mechanophores. These SP doped, thiol-yne-derived stereoelastomers may be synthesized via a base-directed Michael addition polymerization reaction. These linear polymers have been found to be semi-crystalline, recyclable, and mechanochromic under several methods of mechanical activation. Uniaxial tensile tests at three different strain rates confirm that modulus, SH, and SIC behavior depend on the applied strain rate, and therefore are all highly influenced by the viscoelastic effects of chain entanglements. Mechanical and thermal properties are independent of the added SP mechanophore, confirming that the SP mechanophores of the present invention behave as true sensors. The activation strain of SP mechanochromism correlates strongly with the onset of SH.

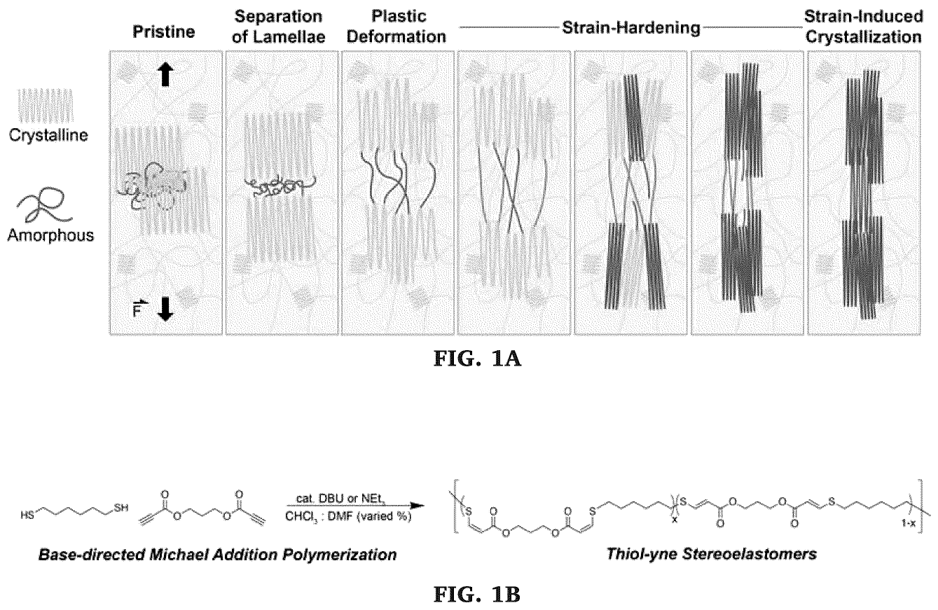

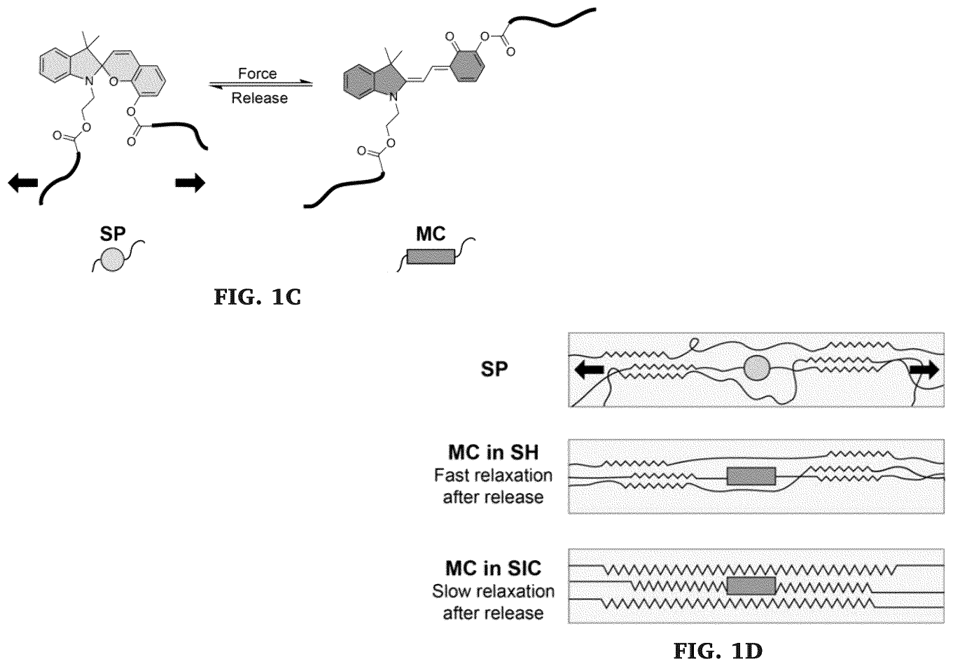

Moreover, the lifetime of the MC state is correlated with the extent of SIC such that, once force is removed, the MC isomer is trapped in a force-activated state until polymer relaxation, as illustrated in FIG. 1D. For a 10-fold decrease in strain rate, there is a proportional decrease in the calculated SP decay kinetic constants. Consequently, these materials show the longest activation lifetime for this SP mechanophore derivative. Stress relaxation tests also indicate correlations between SP activation and SIC.

Further, it has been found that spiropyran recovery kinetics after failure are controlled by strain-induced crystallization, resulting in record activation lifetimes. Advantageously, it has also been found that the mechanochromic responses of the recycled elastomers, which were reset by melting materials and pressing into new films, are consistent with those in pristine films. This further broadens the potential range of applications of these elastomers, including shape memory, which depend on such heat-triggered resetting of elastomer properties.

The following terms may have meanings ascribed to them below, unless specified otherwise. As used herein, the terms “comprising,” “to comprise,” and the like do not exclude the presence of further elements or steps in addition to those listed in a claim. Similarly, the terms “a,” “an” or “the” before an element or feature does not exclude the presence of a plurality of these elements or features, unless the context clearly dictates otherwise.

The phrase “and/or,” as used in the specification and in the claims, should be understood to mean “either or both’ of the elements so conjoined, i.e., elements that are conjunctively present in some cases and disjunctively present in other cases. Multiple elements listed with “and/or” should be construed in the same fashion, i.e., ‘one or more’ of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein, the terms “comprising” “to comprise” and the like, are intended to be open ended and do not exclude the presence of further elements or steps in addition to those listed in a claim or other sentence. For example, a polymer is the to comprise a specific type of linkage if that linkage is present in the polymer, even if other linkages are also present.

Throughout this application, the term “about” is used to indicate that a value includes the inherent variation of error for the device, or the method being employed to determine the value, or the variation that exists among the samples being measured. Unless otherwise stated or otherwise evident from the context, the term “about” means within 10% (i.e., within 10%, 9%, 8%, 7%, 6%, 5%, 4%, 3%, 2%, 1%, or less) above or below the reported numerical value (except where such number would exceed 100% of a possible value or go below 0%). When used in conjunction with a range or series of values, the term “about” applies to the endpoints of the range or each of the values enumerated in the series, unless otherwise indicated. As used in this application, the terms “about” and “approximately” are used as equivalents. Unless otherwise clear from context, all numerical values provided herein in the specification and the claim can be modified by the term “about.”

It should also be understood that the ranges provided herein are a shorthand for all the values within the range and, further, that the individual range values presented herein can be combined to form additional non-disclosed ranges. For example, a range of 1 to 50 is understood to include not only 1 and 50, but any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

Further, any compositions or methods provided herein can be combined with one or more of any of the other compositions and methods provided herein. The fact that given features, elements, or components are cited in different dependent claims does not exclude that at least some of these features, elements, or components maybe used in combination together.

All publications, patent applications, patents, and other references mentioned herein are expressly incorporated by reference in their entirety, which means that they should be read and considered by the reader as part of this text. That the document, reference, patent application, or patent cited in this text is not repeated in this text is merely for reasons of conciseness. In the case of conflict, the present disclosure, including definitions, will control. All technical and scientific terms used herein have the same meaning.

As will be apparent, the term “polymer” is used to refer to a macromolecule having a series of repeated monomer units or, more broadly, to a material made therefrom. Unless otherwise indicated or otherwise clear from the context, the term “polymer” is intended to be interpreted broadly and encompasses all types of polymers, including, but not limited to, homopolymers, copolymers, block copolymers, random copolymers, and other known polymer species. As used herein, the term “homopolymer” refers to a polymer derived from a single monomeric species. And as follows, unless otherwise indicated, the term “copolymer” refers to a polymer derived from two, three or more monomeric species and includes alternating copolymers, periodic copolymers, random copolymers, statistical copolymers and block copolymers.

A polymer “comprises,” is “derived from” or is “derivatized” from a stated monomer if that monomer is incorporated into the polymer. Thus, the incorporated monomer that the polymer comprises is not the same as the monomer prior to incorporation into a polymer, in that at the very least, certain terminal groups are incorporated into the polymer backbone. A polymer is the to comprise a specific type of linkage if that linkage is present in the polymer.

As used herein, the term “residue(s)” is used to refer generally to the part of a monomer or other chemical unit that has been incorporated into a polymer or large molecule. By extension, the terms “residue of the chain transfer agent” and the “chain transfer agent residue” are used interchangeably to refer to the parts of the chain transfer agent that have been incorporated into the bottlebrush polymers. Conversely, a polymer “comprises” or is “derived from” a stated monomer if that monomer is incorporated into the polymer. Thus, the incorporated monomer that the polymer comprises is not the same as the monomer prior to incorporation into a polymer, in that at the very least, certain terminal groups are incorporated into the polymer backbone. A polymer is said to “comprise” a specific type of linkage if that linkage is present in the polymer.

Further, as will be understood by those of skill in the art, the term “elastomer” is used herein to broadly refers to a polymer that has both viscosity and elasticity, or, to combine these words, “viscoelasticity.” These polymers ordinarily comprise polymer chains held together by weak intermolecular forces and will generally exhibit a relatively low Young's modulus and a relatively high yield strength or high failure strain. Similarly, the term “stereoelastomer” is used more narrowly to refer herein to an elastomer having polymer chains that are stereoisomers of each other.

And as will be understood, the term “mechanophore” generally refers to any compound having a particular action or reaction that is triggered by a mechanical force and the narrower terms “mechanochromophore” and “chromophore” used herein interchangeably to refer to a type of mechanophore that undergoes a color change when subjected to a mechanical force. However, unless otherwise indicated or clear from the context, the terms “mechanophore,” “mechanochromophore,” and “chromophore” are used herein interchangeably to refer to a compound that undergoes a color change when subjected to a mechanical force.

All publications, patent applications, patents, and other references mentioned herein are expressly incorporated by reference in their entirety, which means that they should be read and considered by the reader as part of this text. That the document, reference, patent application, or patent cited in this text is not repeated in this text is merely for reasons of conciseness. In the case of conflict, the present disclosure, including definitions, will control. All technical and scientific terms used herein have the same meaning, unless otherwise indicated.

In a first aspect, the present invention is directed to a dipropiolate-derivatized spiropyran (SP) mechanophore, as described below. These SP mechanophores are “dipropiolate-derivatized” in that they have been modified to include two alkyne containing propiolate groups which permit incorporation of these SP mechanophore into the backbone of thiol-yne based elastomers and stereoelatomers, such as those discussed below. The dipropiolate-derivatized spiropyran (SP) mechanophores of the present invention are initially colorless because of their spirocyclic structure. When subjected to sufficient linear strain, however, these SP molecules undergo an electrocyclic ring-opening to form merocyanine (MC), and this isomerization results in a visibly observable blue or purple color due to the increased co-planarity and conjugation of aromatic groups, as illustrated in FIG. 1C. The force-coupled equilibrium between these “off” (SP) and “on” (MC) states can then be monitored using absorbance or fluorescence spectroscopy. (See, e.g., D. A. Davis, A. Hamilton, J. Yang, L. D. Cremar, D. Van Gough, S. L. Potisek, M. T. Ong, P. V. Braun, T. J. Martinez, S. R. White, J. S. Moore, N. R. Sottos, Nature 2009, 459, 68; G. R. Gossweiler, G. B. Hewage, G. Soriano, Q. Wang, G. W. Welshofer, X. Zhao, S. L. Craig, ACS Macro Letters 2014, 3, 216; and Y. Chen, C. J. Yeh, Y. Qi, R. Long, C. Creton, Sci. Adv. 2020, 6, eaaz5093, the disclosures of which are incorporated herein by reference in their entirety). As a result, the SP mechanophores of the present invention are suitable to facilitate macroscopic monitoring of molecular-level stresses within polymer materials.

In various embodiments, the dipropiolate-derivatized spiropyran (SP) mechanophore of the present invention comprises two propiolate functional groups that allows for their insertion in thiol-yne elastomers through a terminal alkyne group on each propiolate functional group via well understood base-directed Michael addition step-growth polymerization reactions, as described below. Each of these propiolate end groups is secured to a central spiropyran molecule by a C₂-C₁₀ alkyl chain and then an ester linkage, as shown in the formula below:

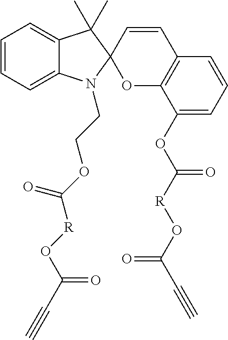

where R is a C₂-C₁₀ alkyl chain.

In some embodiments, each C₂-C₁₀ alkyl (R) chain will be a 2 carbon alkyl chain (C₂H₄), in other embodiments, a 3 carbon alkyl chain (C₃H₆), in other embodiments, a 4 carbon alkyl chain (C₄H₈), in other embodiments, a 5 carbon alkyl chain (C₅H₁₀), in other embodiments, a 6 carbon alkyl chain (C₆H₁₂), in other embodiments, a 7 carbon alkyl chain (C₇H₁₄), in other embodiments, a 8 carbon alkyl chain (C₈H₁₆), in other embodiments, a 9 carbon alkyl chain (C₉H₁₈), and in other embodiments, a 10 carbon alkyl chain (C₁₀H₂₀). In one or more embodiments, the C₂-C₁₀ alkyl chain will be a 5 carbon alkyl chain (C₅H₁₀). In some embodiments, the two C₂-C₁₀ alkyl (R) chains on the dipropiolate-derivatized spiropyran (SP) mechanophore may be the same length. In some other embodiments, the two C₂-C₁₀ alkyl (R) chains on the dipropiolate-derivatized spiropyran (SP) mechanophore may be different lengths.

In some embodiments, the dipropiolate-derivatized spiropyran (SP) mechanophore will have the formula:

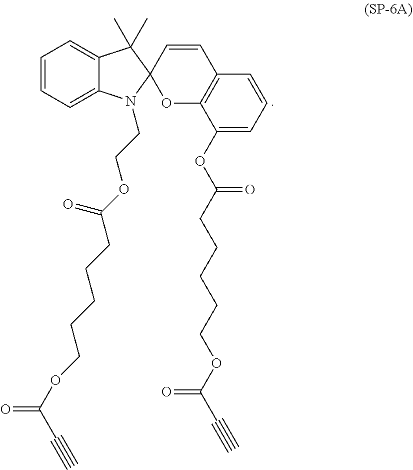 [SMILES: CCC(=O)OCCCCCC(=O)OCCN1c2ccccc2C(C)(C)C12C=Cc1cccc(OC(=O)CCCCCOC(=O)CC)c1O2]

In a second aspect, the present invention is directed to a linear thiol-yne-derived stereoelastomer comprising the dipropiolate-derivatized spiropyran (SP) mechanophore, described above. As set forth above, the dipropiolate-derivatized SP mechanophores of the present invention contain propiolate end groups and can be easily added to thiol-yne-derived stereoelastomer via well understood base-directed Michael addition step-growth polymerization reactions. Because these linear thiol-yne-derived stereoelastomer have the dipropiolate-derivatized SP mechanophores described above integrated into their polymer chain, the stereoelastomers also exhibit strain induced mechanochromatism. Advantagously, at least in small or moderate quantities, these SP mechanophores do not appear to have any effect on the mechanical or thermal properties of the thio-yne derived stereoelastomers of the present invention.

In various embodiments, the thiol-yne derived stereoelastomers of the present invention are linear polymers comprising the reaction product of two or more a linear C₂-C₂₀ dithiol monomers, two or more linear C₂-C₁₀ dipropiolate monomers, and a relatively small amount of the dipropiolate-derivatized SP mechanophore monomer, described above. As will be appreciated by those of skill in the art, and is discussed in more detail below, when these monomers are combined, the thiol end groups of the dithiol monomer will react with the alkyne end groups of the terminal propiolate groups on the dipropiolate and SP mechanophore monomers, to form a thiol-yne elastomer material containing the residues of all three types of monomers. In this reaction, a bond is formed between each thiol end group and each alkyne group by alkylating the triple bond of the alkyne to form stereospecific isomers, with each resulting double bond providing either a cis or a trans stereoisomer. Accordingly, the thiol-yne elastomer compositions of the present invention will have a combination of cis and trans configurations throughout its length and, as will be discussed in more detail below, the relative number of these cis and trans configurations has a significant effect on the overall structure and the resulting properties of these thiol-yne polymers.

It has been found, further, that the ordering of the polymer chains into micro-line domains in the thiol-yne elastomers according to various embodiments of the present invention is enhanced by high cis-content, in line with that of natural rubber and a decrease in the % cis content corresponds to a decreased crystallinity within these thiol-yne elastomers. Further, the semicrystalline nature of these elastomer networks manifests itself as a significant improvement of the network's mechanical properties with an increase in the fraction of the cis-isomers.

In some embodiments, the dipropiolate residues will be the residue of a C₂ to C₈ dipropiolate monomer, in other embodiments, a C₂ to C₆ dipropiolate monomer, in other embodiments, a C₂ to C₅ dipropiolate monomer, in other embodiments, a C₂ to C₄ dipropiolate monomer, in other embodiments, a C₂ or C₃ dipropiolate monomer, in other embodiments, a C₂ to Cₓ dipropiolate monomer, in other embodiments, a C₃ to Cₓ dipropiolate monomer, and in other embodiments, a C₃ to C₅ dipropiolate monomer. In some embodiments, the dipropiolate residues will be the residue of a propane-1,3-diyl dipropiolate monomer.

In some embodiments, the dipropiolate residues will be the residue of a dipropiolate monomer having the formula:

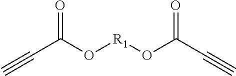

where R₁ is a C₂ to C₈ alkyl group. In some embodiments, the dipropiolate residues will be the residue monomer having the formula:

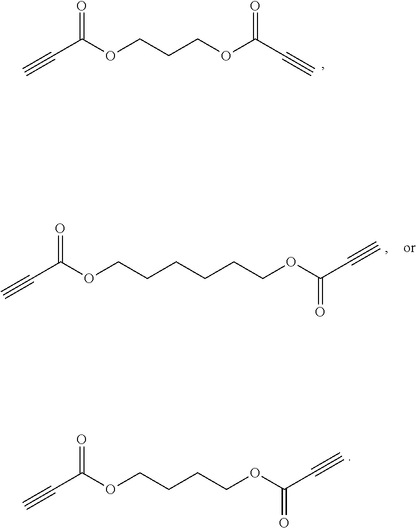

In some embodiments, the linear dithiol residues will be the residue of a C₂ to C₂₀ linear dithiol monomer, in other embodiments, a C₂ to C₁₈ linear dithiol monomer, in other embodiments, a C₂ to C₁₆ linear dithiol monomer, in other embodiments, a C₂ to C₁₄ linear dithiol monomer, in other embodiments, a C₂ to C₁₀ linear dithiol monomer, in other embodiments, a C₂ to C₆ linear dithiol monomer, in other embodiments, a C₂ to C₆ linear dithiol monomer, in other embodiments, a C₂ to C₂₀ linear dithiol monomer, in other embodiments, a C₂ to C₄ linear dithiol monomer, in other embodiments, a C₄ to C₂₀ linear dithiol monomer, in other embodiments, a C₆ to C₂₀ linear dithiol monomer, in other embodiments, a C₈ to C₂₀ linear dithiol monomer, in other embodiments, a C₁₀ to C₂₀ linear dithiol monomer, in other embodiments, a C₁₂ to C₂₀ linear dithiol monomer. In some embodiments, the linear C₂-C₂₀ dithiol residues will be the residue of 1,6-hexanedithiol. Here, as well as elsewhere in the specification and claims, individual range values can be combined to form additional non-disclosed ranges.

In some embodiments, the linear dithiol residues will be the residue of a dithiol monomer having the formula:

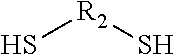

where R₂ is a C₂-C₂₀ alkyl group, an aryl group, an ether group, or a poly(ethylene glycol).

In one or more embodiments, linear dithiol residues will have the formula:

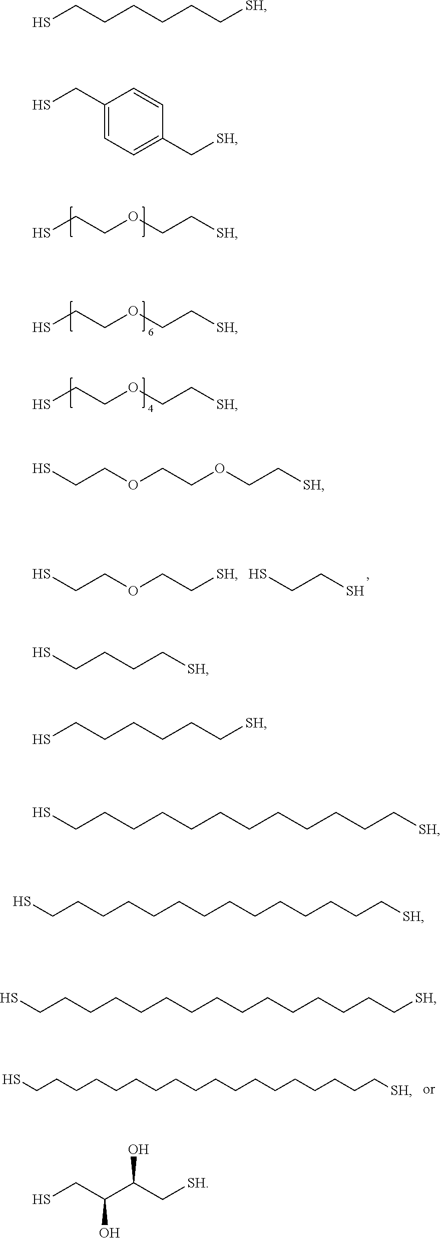

As set forth above, it has been found that the ratio cis double bonds to trans double bonds in the resulting thiol-yne elastomer composition of the biomimetic synthetic rubber compositions of the present invention may be tuned by changing the polarity of the reaction solvent or solvent combination used to dissolve the multi-functional alkyne monomer and multi-functional thiol monomer being used, without altering the molecular weight of the polymer formed. In general, it has been found that in embodiments where a more polar solvent is used a thiol-yne elastomer composition having a higher ratio of cis double bonds to trans double bonds is produced and conversely, in embodiments wherein a less polar solvent is used, the resulting thiol-yne elastomer compositions had a lower ratio of cis double bonds to trans double bonds. In this manner, it has been found, for example, that thiol-yne elastomer materials can be synthesized from dialkyne and dithiol monomers with cis-trans double bond ratios that can be tuned between 22% cis and 80% cis based on the polarity of the reaction solvent. Here, as well as elsewhere in the specification and claims, individual range values can be combined to form additional non-disclosed ranges.

In one or more embodiments, the polarity of the reaction solvent (the solvent or solvent combination that dissolves the multi-functional alkyne monomer and multi-functional thiol monomer may be controlled by using a solvent combination containing two or more co-solvents for the multi-functional alkyne monomer and multi-functional thiol monomer having differing polarities and then controlling the ratio of those solvents to obtain the desired polarity. In some embodiments, the solvent combination used may include both polar and non-polar solvents. Suitable polar/non-polar solvent combinations may include, without limitation, chloroform (CHCl₃) and N,N-dimethylformamide (DMF), chloroform and N-methylpyrolidone (NMP). It has been found, for example, that by varying the ratio of CHCl₃ to DMF used as the reaction solvent, is it possible to vary the cis/trans ratio of the resulting polymer without affecting its molecular weight. That is, a higher percentage of CHCl₃ in the reaction solvent combination will give polymer having a lower % cis and a higher percentage of DMF in the reaction solvent combination will give a polymer having approximately the same molecular weight and a higher % cis using the same base catalyst.

In one or more embodiments, the reaction solvent or solvent combination may have a relative polarity from about 0.2 or more to about 0.4 or less. The values for relative polarity are normalized from measurements of solvent shifts of absorption spectra and were extracted from Christian Reichardt (Solvents and Solvent Effects in Organic Chemistry, Wiley-VCH Publishers, 3rd ed., 2003), the disclosure of which is incorporated herein by reference. DMF, for example, has a relative polarity of 0.386 and CHCl₃, has a relative polarity of 0.259.

In some embodiments, 70% or more of the stereoactive alkene bonds in the linear thiol-yne-derived stereoelastomers of the present invention are in a cis orientation. In some embodiments, 75% or more of the stereoactive alkene bonds in the linear thiol-yne-derived stereoelastomers of the present invention are in a cis orientation. In some other embodiments, 80% or more of the stereoactive alkene bonds in the linear thiol-yne-derived stereoelastomers of the present invention are in a cis orientation. In some embodiments, 85% or more of the stereoactive alkene bonds in the linear thiol-yne-derived stereoelastomers of the present invention are in a cis orientation. In some embodiments, 90% or more of the stereoactive alkene bonds in the linear thiol-yne-derived stereoelastomers of the present invention are in a cis orientation. Here, as well as elsewhere in the specification and claims, individual range values can be combined to form additional non-disclosed ranges.

In some embodiments, the stereoelstomers of the present invention will comprise an about 1:1 ratio of dithiols and dipropriolate residues. In some of these embodiments, the stereoelstomers of the present invention will comprise an about 1:1 ratio of 1,6-hexanedithiol monomer residues to propane-1,3-diyl dipropiolate monomer residues.

In various embodiments, the linear thiol-yne-derived stereoelastomer of the present invention will contain from about 0.001 wt. % to about 5 wt. %, preferably from about 0.01 wt. % to about 2.0 wt. %, and more preferably from about 0.8 wt. % to about 1.6 wt. % of said the dipropiolate-derivatized SP mechanophores described above. In some embodiments, the linear thiol-yne-derived stereoelastomer of the present invention will contain from about 0.001 wt. % to about 4.5 wt. % , in other embodiments, from about 0.001 wt. % to about 4 wt. %, in other embodiments, from about 0.001 wt. % to about 3.5 wt. %, in other embodiments, from about 0.001 wt. % to about 3 wt. %, in other embodiments, from about 0.001 wt. % to about 2.5 wt. %, in other embodiments, from about 0.001 wt. % to about 2 wt. %, in other embodiments, from about 1 wt. % to about 5 wt. %, in other embodiments, from about 2 wt. % to about 5 wt. %, and in other embodiments, from about 3 wt. % to about 5 wt. %. Here, as well as elsewhere in the specification and claims, individual range values can be combined to form additional non-disclosed ranges. In some embodiments, these elastomers will contain about 1.0 wt. % of the dipropiolate-derivatized SP mechanophores. In some other embodiments, these elastomers will contain about 1.5 wt. % of the dipropiolate-derivatized SP mechanophores.

In various embodiments, the linear thiol-yne-derived stereoelastomer will have the formula:

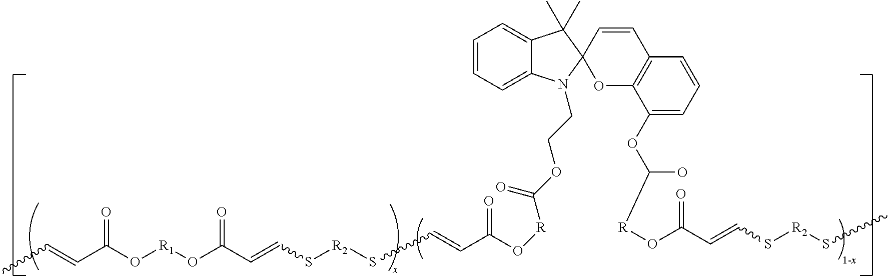

where x is a mole fraction from about 0.95 to about 0.999, preferably from about 0.980 to about 0.999, and more preferably from about 0.992 to about 0.996; R is a C₂-C₁₀ alkyl group; R₁ is a C₂ to C₈ alkyl group; R₂ is a C₂-C₂₀ alkyl group, an aryl group, an ether group, or a poly(ethylene glycol); and [symbol] indicates a stereoactive bond (i.e. it may be cis or trans). In some embodiments, x is a mole fraction from about 0.960 to about 0.999, in other embodiments, from about 0.970 to about 0.999, in other embodiments, from about 0.980 to about 0.999, in other embodiments, from about 0.990 to about 0.999, in other embodiments, from about 0.995 to about 0.999, in other embodiments, from about 0.950 to about 0.995, in other embodiments, from about 0.950 to about 0.990, in other embodiments, from about 0.950 to about 0.980, and in other embodiments, from about 0.950 to about 0.970. Here, as well as elsewhere in the specification and claims, individual range values can be combined to form additional non-disclosed ranges. In some embodiments, x is a mole fraction of about 0.995, R is a C₅ alkyl group; R₁ is a C₃ alkyl group and R₂ is a C₆ alkyl group.

In some of these embodiments, the linear thiol-yne-derived stereoelastomer will have the formula:

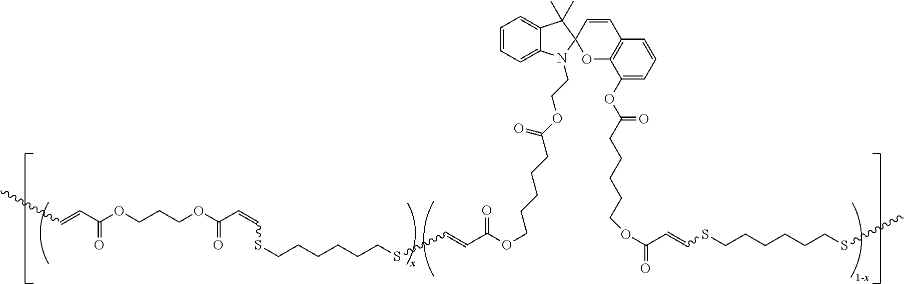

where x is a mole fraction of from about 0.95 to about 0.999 and [symbol] indicates a stereoactive bond. In various embodiments, x is a mole fraction of from about 0.96 to about 0.999, preferably from about 0.980 to about 0.999, and more preferably from about 0.992 to about 0.996. In some embodiments, x is a mole fraction of about 0.995. Here, as well as elsewhere in the specification and claims, individual range values can be combined to form additional non-disclosed ranges.

In one or more embodiments, the linear thiol-yne-derived stereoelastomer have a Young's modulus of from about 70 MPa to about 150 MPa. In some embodiments, the linear thiol-yne-derived stereoelastomer have a Young's modulus of from about 70 MPa to about 140 MPa, in other embodiments, from about 70 MPa to about 120 MPa, in other embodiments, from about 70 MPa to about 100 MPa, in other embodiments, from about 70 MPa to about 90 MPa, in other embodiments, from about 70 MPa to about 80 MPa, in other embodiments, from about 90 MPa to about 150 MPa, in other embodiments, from about 110 MPa to about 150 MPa, in other embodiments, from about 130 MPa to about 150 MPa, and in other embodiments, from about 140 MPa to about 150 MPa. Here, as well as elsewhere in the specification and claims, individual range values can be combined to form additional non-disclosed ranges.

As set forth above, these elastomers contain amorphous and crystalline areas and undergo strain hardening under axial strain. It has been found that at or about the point of strain hardening, the SP mechanophores in these elastomers, will open and for the merocyanine (MC) form of the mechanophore, as shown in Scheme 1 below. When this occurs, the polymer will exhibit a visible blue color.

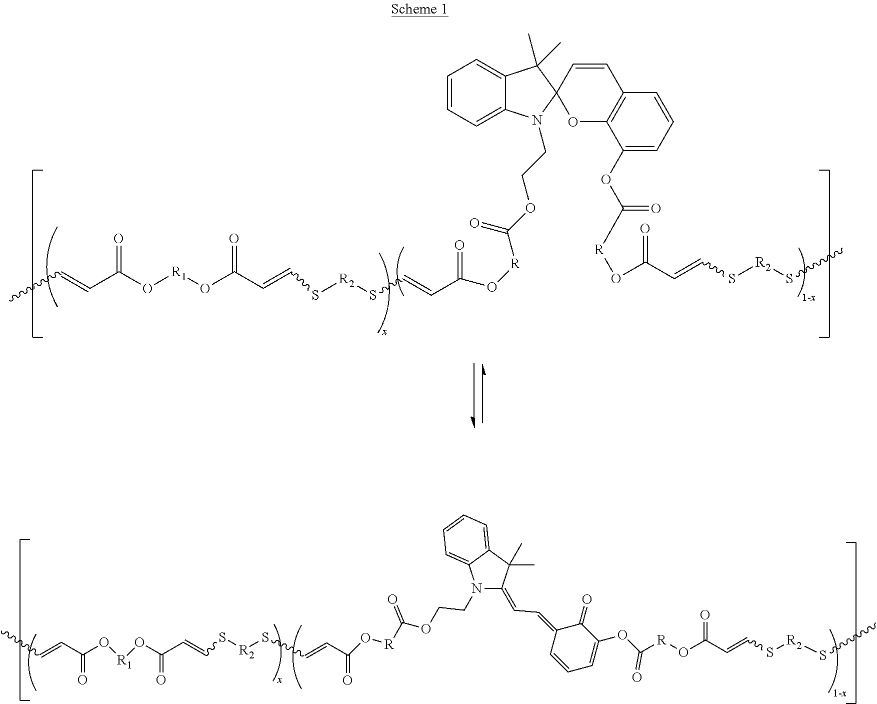

In addition, because these linear thiol-yne-derived stereoelastomers are not chemically crosslinked, they have been found to have the same properties even after melt recycling.

In a third aspect, the present invention is directed to a method for making the dipropiolate-derivatized SP mechanophore described above. In various embodiments, the dipropiolate-derivatized SP mechanophore described above may be made as shown in Scheme 2, below.

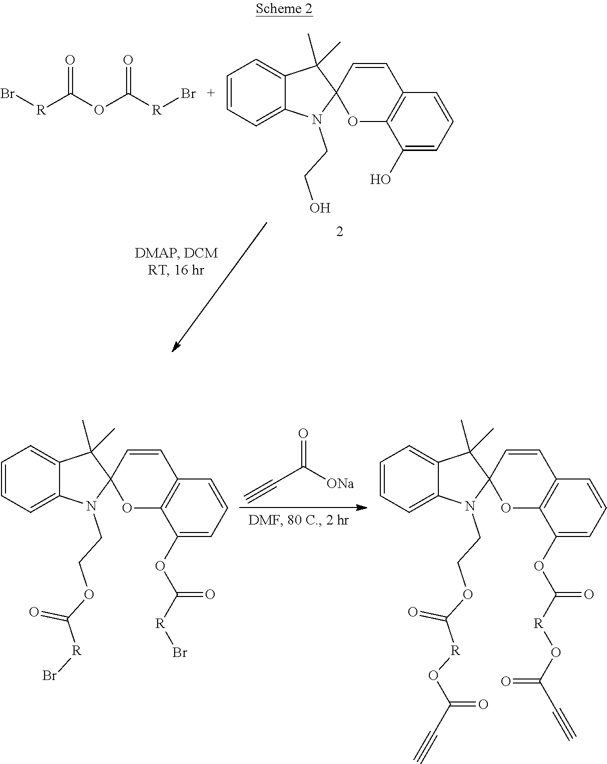

where R is a C₂-C₁₀ alkyl group.

In one or more of these embodiments, the method for making the dipropiolate-derivatized SP mechanophore described above comprises the steps of: dissolving a SP diol and an organic base in a suitable solvent; adding a quantity of 6-bromohexanoic anhydride to the solution of step A and stirring for from about 6 to about 18 hours to produce 2-(8-((6-bromohexanoyl)oxy)-3′,3′-dimethylspiro [chromene-2,2′-indolin]-1′-yl) ethyl 6-bromohexanoate; placing the 2-(8-((6-bromohexanoyl)oxy)-3′,3′-dimethylspiro [chromene-2,2′-indolin]-1′-yl) ethyl 6-bromohexanoate in a suitable reaction vessel in a dark location and adding sodium propiolate; sealing the reaction vessel and heating it to the curing temperature of the polymer for sufficient time to cure the polymer; collecting and purifying the reaction product to produce the 3′,3′-dimethyl-1′-(2-((6-(propioloyloxy)hexanoyl) oxy)ethyl) spiro[chromene-2,2′-indolin]-8-yl 6-(propioloyloxy)hexanoate.

In one or more embodiments, the spiropyran (SP) diol has the formula:

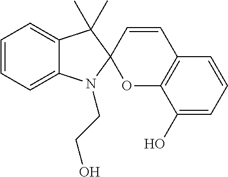 [SMILES: CC1(C)c2ccccc2N(CCO)C12C=Cc1cccc(O)c1O2]

The organic base used is not particularly limited and any suitable organic base of sufficient strength may be used provided that the solvent chosen does not damage the reagents or inhibit the reaction. In one or more of these embodiments, the organic base is N,N′-dimethylamino pyridine (DMAP). Similarly, the solvent used to dissolve the SP diol and the organic base is not particularly limited provided that the solvent chosen does not damage the reagents or inhibit the reaction. In some embodiments, the solvent is dichloromethane.

In some embodiments, the step of adding a quantity of 6-bromohexanoic anhydride further comprises: concentrating and then redissolving the 2-(8-((6-bromohexanoyl)oxy)-3′,3′-dimethylspiro [chromene-2,2′-indolin]-1′-yl) ethyl 6-bromohexanoate in ethyl acetate; washing the solution with water, sodium bicarbonate, and brine and then drying it on sodium sulfate; and concentrating the product onto neutral aluminum oxide and then purifying it via column chromatography.

In some embodiments, the step of collecting and purifying comprises: transferring the reaction product into saturated NH₄Cl (200 mL) and stirring; extracting the crude product with ethyl acetate and then washing it in water and brine and drying it over Na₂SO₄; filtering and concentrated the crude product onto silica gel, and then drying it under vacuum; and purifying the product by flash chromatography.

In a fourth aspect, the present invention is directed to a method for making the linear thiol-yne-derived stereoelastomers described above. In one or more embodiments, these linear thiol-yne-derived stereoelastomers may be made as set forth in Scheme 3, below.

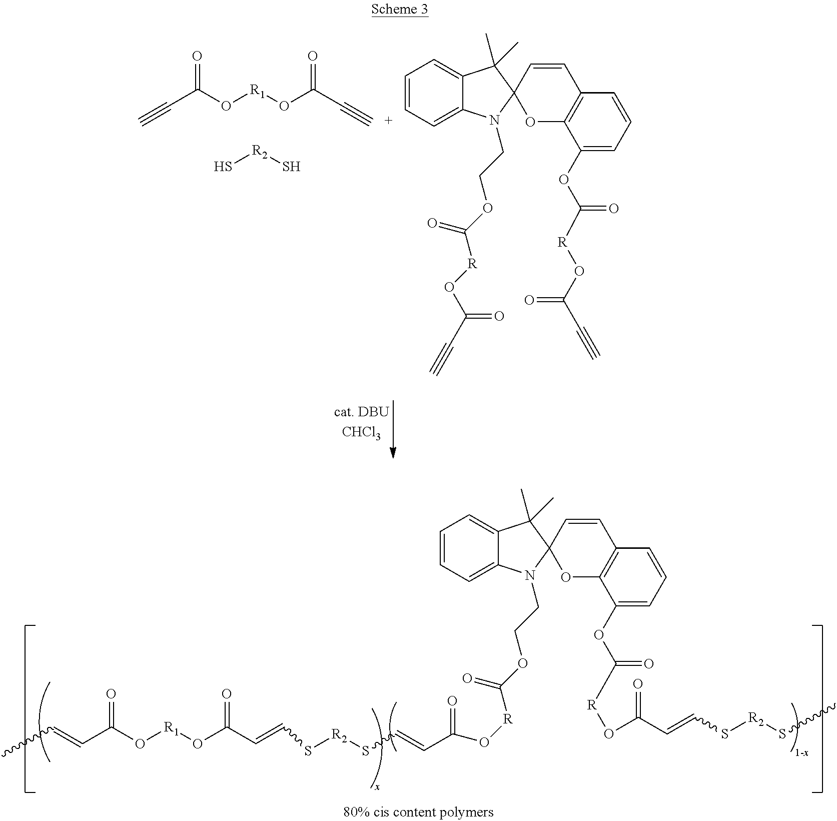

where x is a mole fraction from about 0.95 to about 0.999, preferably from about 0.980 to about 0.999, and more preferably from about 0.992 to about 0.996; R is a C₂-C₁₀ alkyl chain; R₁ is a C₃ alkyl group; and R₂ is a C₆ alkyl group; and [symbol] indicates a stereoactive bond.

In some embodiments, the method for making the thiol-yne-derived stereoelastomer comprises: combining a dipropiolate-derivatized SP mechanophore as described above, a C₂-C₂₀ dithiol such as 1,6-hexanedithiol, and a first quantity of a C₂-C₈ dipropiolate such as propane-1,3-diyl dipropiolate in a suitable reaction vessel; cooling the reaction vessel to a temperature of from about −20° C. to about −5° C. and stirring for from about 15 min to about 60 min; separately combining 1,8-diazabicyclo[5.4.0]undec-7-ene (DBU) and a polar solvent or solvent combination as set forth above and slowly adding the combination to the reaction vessel; allowing the reaction vessel to warm to room temperature and after from about 15 to about 45 min, dissolving a second quantity of the C₂-C₈ dipropiolate in a suitable solvent; adding the dipropiolate solution to the reaction vessel to end-cap any unreacted thiol groups; adding a radical scavenger to the reaction vessel to prevent crosslinking; and collecting a purifying the reaction product to produce the thiol-yne-derived stereoelastomer described above.

In these embodiments, the dithiol and dipropriolate may be any of those described above. In some of these embodiments, dithiol is 1,6-hexanedithiol and the dipropiolate is propane-1,3-diyl dipropiolate. In some embodiments, the step of slowly adding comprises adding to the DBU solution dropwise over a period of about 20 min. The radical scavenger used is not particularly limited and any suitable radical scavenger may be used. In some embodiments, the radical scavenger is butylated hydroxytoluene (BHT)

In some of these embodiments, the step of collecting and purifying further comprises: precipitating the product into Et₂O; collecting the thiol-yne-derived stereoelastomer by decanting the supernatant; and drying the product of step 2 under vacuum for from about 12 to about 36 hours to produce the purified polymer thiol-yne-derived stereoelastomer of the present invention.

In another aspect, the present invention is directed to a film comprising the linear thiol-yne-derived stereoelastomer described above. In some embodiments, these linear thiol-yne-derived stereoelastomer may be fabricated using a melt-compression machine. It has been found that the combination of melting, adding intense pressure, then slowly cooling under confinement resets the thermal history, resulting in consistent crystallinity within the elastomer film. In some of these embodiments, the melt-press machine is pre-heated to 10° C. above the polymer melting point (Tₘ), and the bulk polymer is added into a 50×50×1 mm mold and placed on the compression machine under ambient air and pressure. After 15 min of melting, the polymer is then compressed under about 100 psi of pressure for about 20 min. The film is then slowly cooled to room temperature over about 20 h, while under 100 psi of pressure to prevent wrinkling of the film's surface.

### EXPERIMENTAL

In order to better described and further reduce them to practice, examples of the dipropiolate-derivatized spiropyran (SP) mechanophore and the SP doped thiol-yne-derived stereoelastomers of the present invention were synthesized and tested to explore their mechanical and mechanochromophoric properties and suitability for sensor, strain-sensing and shape-memory applications.

### 1.1. Synthesis

SP monomer 4 was synthesized according to Scheme 4, below. Commercially available 6-bromohexanoic acid was used to synthesize the 6-bromohexanoic anhydride 1 in quantitative yield, in a manner analogous to a previously reported synthesis using N,N′-diisopropylcarbodiimide. (See, R. K. Pathak, S. Dhar, Chem. Eur. J. 2016, 22, 3029, the disclsoure of which is incorporated herein by reference in its entirety). The SP diol 2 was synthesized using a previously published procedure. (See, M. H. Barbee, T. Kouznetsova, S. L. Barrett, G. R. Gossweiler, Y. Lin, S. K. Rastogi, W. J. Brittain, S. L. Craig, J. Am. Chem. Soc. 2018, 140, 12746, the disclsoure of which is incorporated herein by reference in its entirety).The functional 6-bromohexanoic anhydride 1 was then coupled onto the SP diol 2 using a catalytic amount of N,N′-dimethylamino pyridine (DMAP), to yield intermediate molecule 3 (68%). Sodium propiolate was synthesized according to procedures published previously, (see, J. Yang, K. Hong, P. V. Bonnesen, Journal of Labelled Compounds and Radiopharmaceuticals 2011, 54, 743, the disclsoure of which is incorporated herein by reference in its entirety) and then used in excess with intermediate molecule 3 in anhydrous DMF to yield the dipropiolate-functionalized SP monomer 4 via nucleophilic substitution (36%).

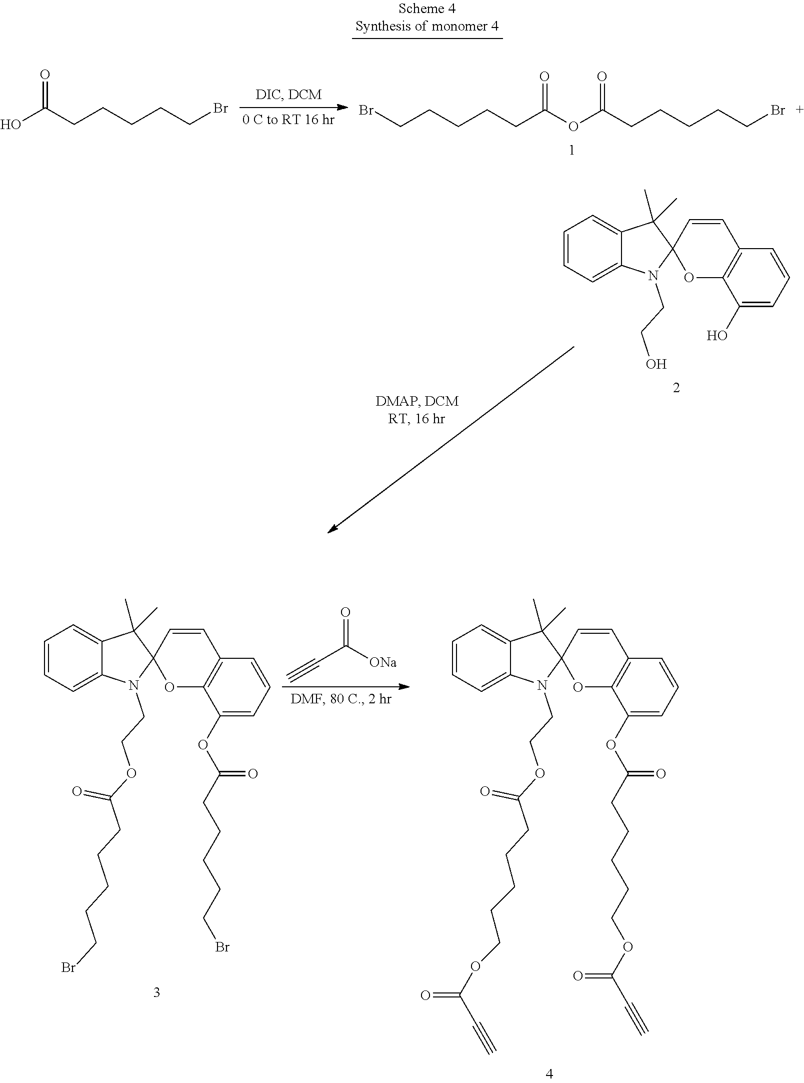

Thiol-yne polymers were synthesized analogously to previous reports, through a base-directed thiol-yne step growth polymerization using dithiol and dipropiolate monomers 5 and 6, and a small mole fraction of the dipropiolate-functionalized SP monomer 4 (See, Scheme 5, below.). (See, e.g., C. A. Bell, J. Yu, I. A. Barker, V. X. Truong, Z. Cao, A. V. Dobrynin, M. L. Becker, A. P. Dove, Angew. Chem. Int. Ed. 2016, 55, 13076, J. C. Worch, A. C. Weems, J. Yu, M. C. Arno, T. R. Wilks, R. T. R. Huckstepp, R. K. O'Reilly, M. L. Becker, A. P. Dove, Nat Commun 2020, 11, 3250, and M. B. Wandel, C. A. Bell, J. Yu, M. C. Arno, N. Z. Dreger, Y. H. Hsu, A. Pitto-Barry, J. C. Worch, A. P. Dove, M. L. Becker, Nat Commun 2021, 12, 446, the disclsoures of which are incorporated herein by reference in their entirety).

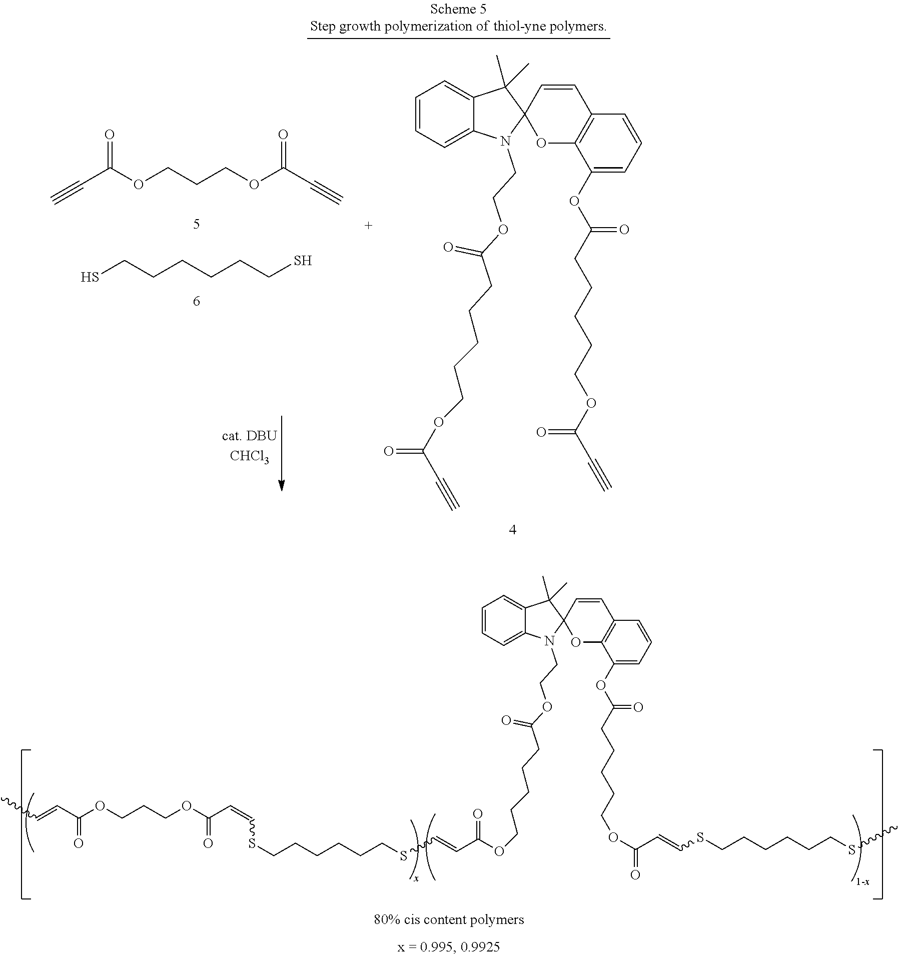

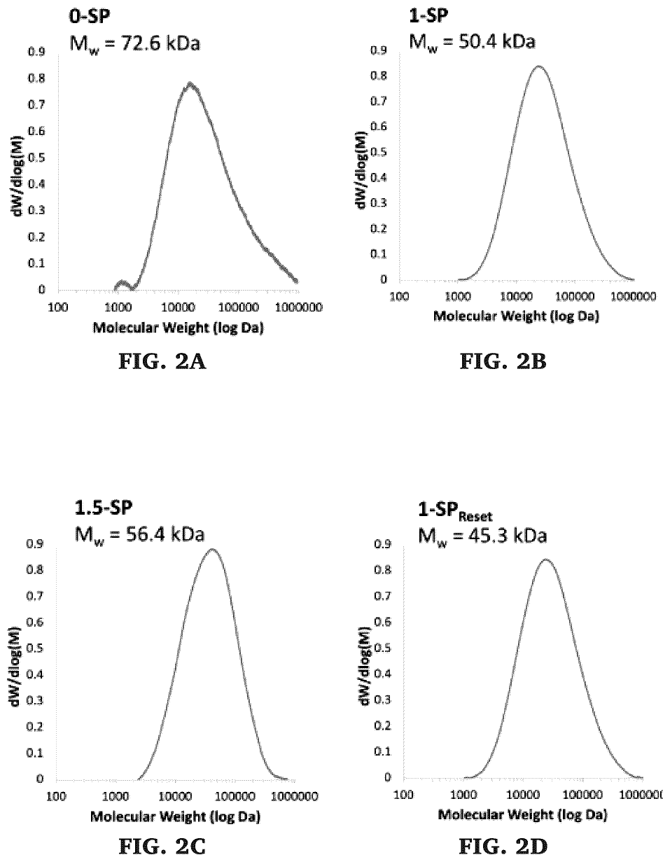

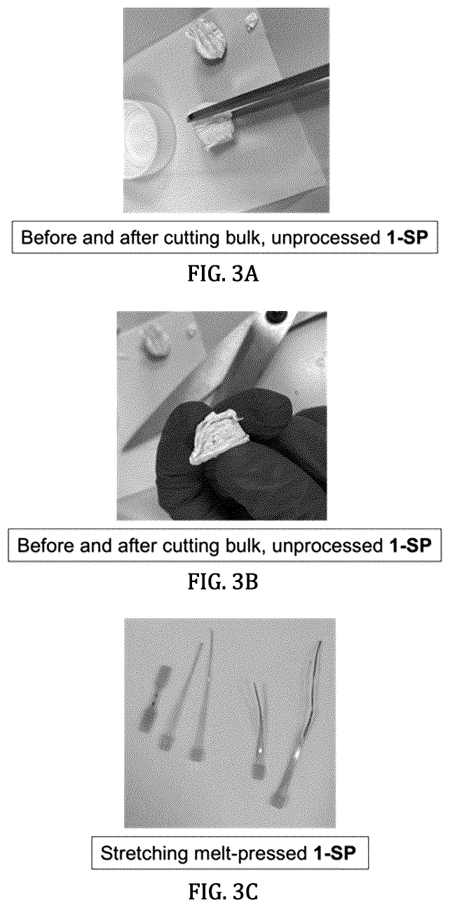

Monomer stoichiometry was controlled precisely so that the molar equivalences of propiolate monomer were in a 1:1 ratio to the added dithiol. The amount of added 4 spanned 0, 1, and 1.5 wt % of the bulk, resulting in polymers named 0-SP, 1-SP, and 1.5-SP, respectively (Table 1); for 1-SP and 1.5-SP, this corresponds to 0.25 and 0.38 mol % of 4. Under these synthetic conditions, polymers of 40-70 kDa (weight-averaged molecular weight, M_{w}) (see, FIGS. 2A-D) and 79.9±0.3% cis content (average±1 standard deviation, n=3; calculated from the integrations of the cis and trans ¹H NMR resonances) along the polymer backbone were obtained consistently. (See, e.g., C. A. Bell, J. Yu, I. A. Barker, V. X. Truong, Z. Cao, A. V. Dobrynin, M. L. Becker, A. P. Dove, Angew. Chem. Int. Ed. 2016, 55, 13076, the disclsoure of which is incorporated herein by reference in its entirety). 1-SP and 1.5-SP are mechanochromic in their bulk unprocessed state, and dipropiolate-functionalized SP monomer 4 is activated under several forms of applied force (FIG. 3A-C). Notably, these polymers may be recycled multiple times by melt-pressing them into new films, as they are not cross-linked.

TABLE 1
Polymer nomenclature and characterization.

| | SP content | | M_{w} | T_{g} | \dot{ε} | | Ε | break |
|---|---|---|---|---|---|---|---|---|
| Polymer | [wt %] | [mol %] | [kg mol⁻¹] | [° C.] | [mm min⁻¹] | [s⁻¹]* | [MPa] | [%] |
| 0-SP | — | — | 72.6 | −0.8 | 10 | 0.02 | 128.6 ± 7.1 | 599 ± 21 |
| | | | | | 1 | 0.002 | 115.6 ± 3.4 | 683 ± 61 |
| | | | | | 0.1 | 0.0002 | 96.9 ± 5.7 | 751 ± 51 |
| 1-SP | 1.0 | 0.25 | 50.4 | 0.1 | 10 | 0.02 | 130.0 ± 9.0 | 781 ± 58 |
| | | | | | 1 | 0.002 | 104.3 ± 5.8 | 826 ± 64 |
| | | | | | 0.1 | 0.0002 | 97.9 ± 2.3 | 880 ± 38 |
| 1.5-SP | 1.5 | 0.38 | 56.4 | 2.3 | 10 | 0.02 | 106.6 ± 4.0 | 967 ± 18 |
| | | | | | 1 | 0.002 | 89.3 ± 3.1 | 995 ± 52 |
| | | | | | 0.1 | 0.0002 | 82.3 ± 4.9 | 974 ± 39 |

*Approximate values; see Table 4 (Appendix) for averages and associated gauge lengths; see also FIGS. 2A-D.

### 1.2. Film Fabrication

Films (0.75-1.00 mm) of each elastomer were fabricated using a melt-compression machine. The combination of melting, adding intense pressure, then slowly cooling under confinement resets the thermal history, resulting in consistent crystallinity within the elastomer film. The melt-press machine was pre-heated to 10° C. above the polymer melting point (Tₘ), then bulk polymer was added into a 50×50×1 mm mold and placed on the compression machine under ambient air and pressure. After 15 min of melting, the polymer was compressed under 100 psi of pressure for 20 min. Subsequently, each film was slowly cooled to room temperature over 20 h, while under 100 psi of pressure to prevent wrinkling of the film's surface. The films were visually inspected to ensure that no bubbles were present in the films, then dog-bone tensile bars were cut using a custom ASTM Die D-638 Type V die punch (neck dimensions 9.53 mm×1.50 mm). Two black ink dots were placed on the surface of each sample to assist in image processing, but they were not used for any measurements. All films were pressed to a similar thickness (0.75-1.00 mm) to facilitate direct comparison of all image sets.

### 1.3. Tensile Tests at Different Strain Rates

Samples were tested at 3 different strain rates (\dot{ε}, s⁻¹), corresponding to crosshead speeds of 10, 1, and 0.1 mm min⁻¹ (herein, referred as \dot{ε}₁₀, \dot{ε}₁, and \dot{ε}_{0.1}). The large deformations and heterogeneous response (e.g., necking) observed in these samples complicates any evaluation of intrinsic true stress/true strain properties, and it is important to regard the reported stress/strain behavior in this context. As discussed below, however, the \dot{ε}-dependent behavior at large deformations that is the focus here is well characterized by governing equations of uncross-linked polymeric materials, even across variations in sample size and initial strain rate. The sample gauge lengths (mm) and the corresponding initial strain rates (2×10⁻⁴−2×10⁻² s⁻¹) are listed in Table 4 (Appendix). All tests were done in triplicate to ensure reproducibility, and results are reported as average±1 standard deviation (n=3), unless otherwise noted.

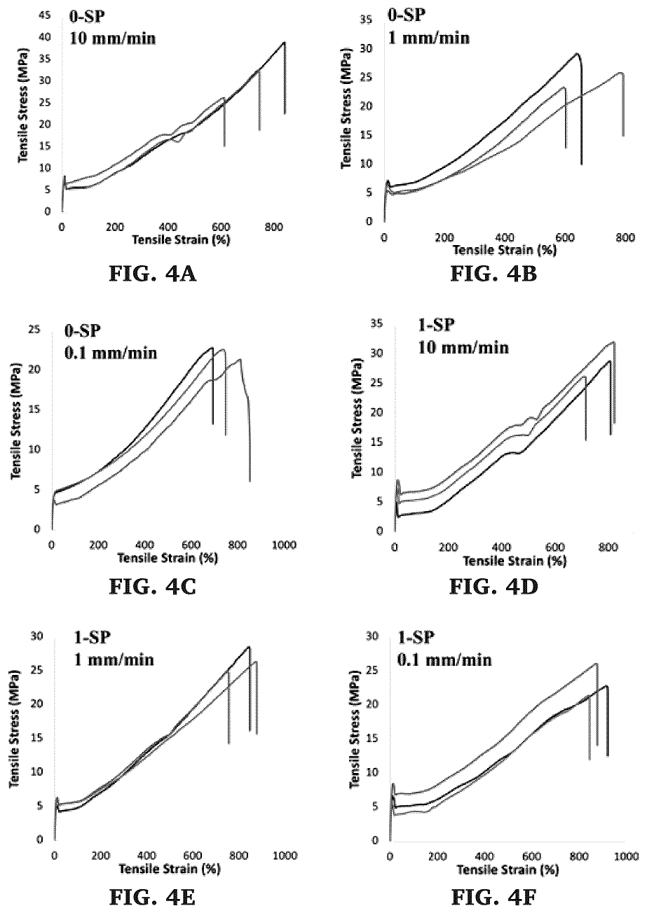

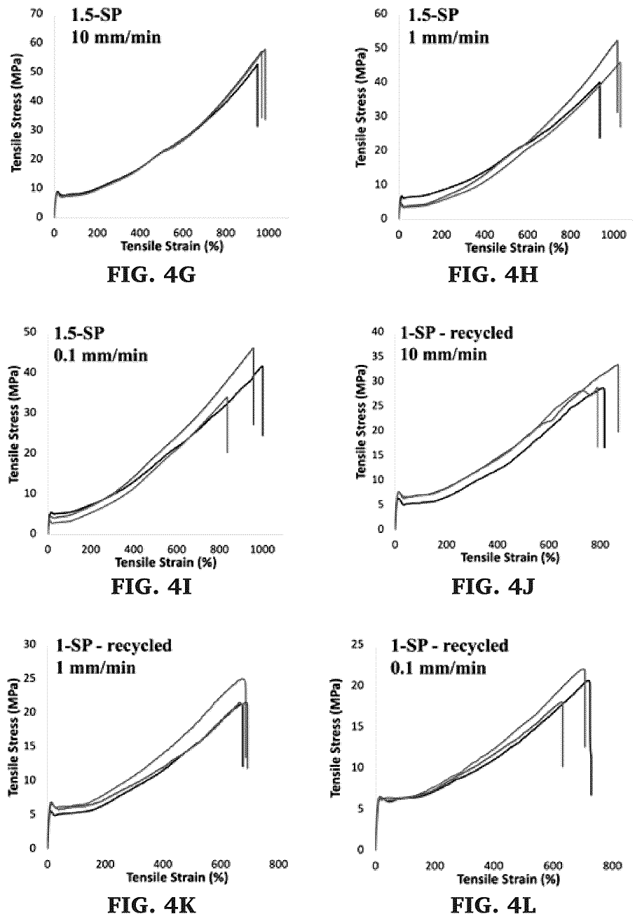

Tensile mechanical data (Table 2 (Appendix) and FIGS. 4A-L) for these polymers typically show a short interval of elastic deformation before yielding at around 5-9 MPa and 10-15% elongation (λ). This is followed by a regime of strain-softening and plastic deformation. Within the ranges examined here, higher \dot{ε} results in larger moduli (E), and, in most cases, lower λ at break (Table 1). When stretched to approximately λ=200%, polymers enter SH regimes, then subsequently fail around λ=600-1000% and 30-50 MPa stress. Typically, tensile testing data display two SH regimes that are separated by a brief interval of strain-softening. This interval is most pronounced for \dot{ε}₁₀-tested samples; when stretched slowly, there is a more gradual progression through the SH regimes.

### 1.3.1. Effect of Strain Rate

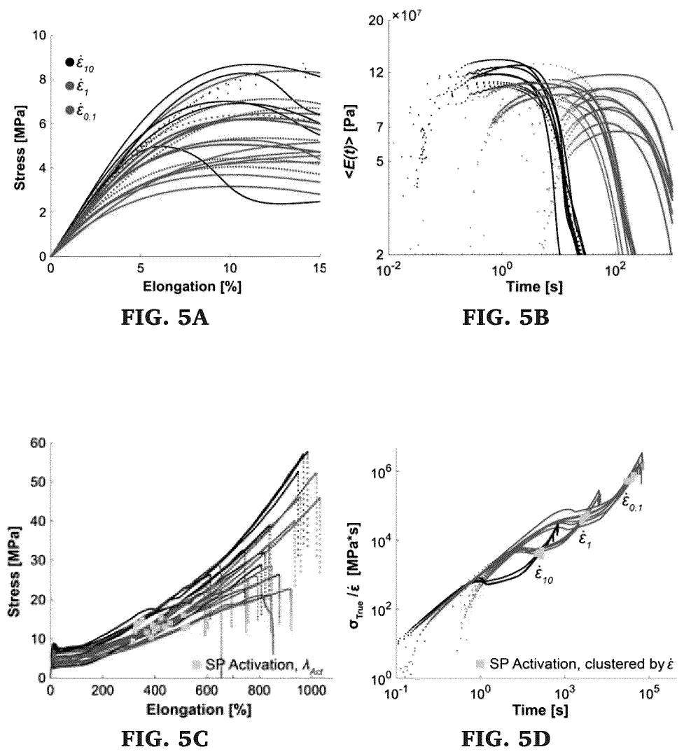

In FIG. 5A, stress is plotted as a function of λ at early deformations for 0-SP, 1-SP, and 1.5-SP, across all rates. In general, stress at a given strain decreases with \dot{ε}, which is expected for networks without chemical cross-links. (See, e.g., Walter Kauzmann, H. Eyring, J. Am. Chem. Soc. 1940, 62, 3113 and X. Hu, J. Zhou, W. F. M. Daniel, M. Vatankhah-Varnoosfaderani, A. V. Dobrynin, S. S. Sheiko, Macromolecules 2017, 50, 652, the disclosures of which are incorporated herein by reference in their entirety). Consequently, the time-average network Young's modulus, [symbol] E(t) [symbol] , was calculated to examine the scaling effects of the applied \dot{ε} on mechanical behavior. At a given \dot{ε}, the tensile stress (σ) decays as a function of time according to Equation (1):

σ(t)=∫₀^{t}E(t−t′)dλ(t′)=λ(t) [symbol] E(t) [symbol] (1)

where λ(t) is the strain at any given time (λ(t)=\dot{ε}t) . Algebraic manipulation of Equation (1) reveals that [symbol] E(t) [symbol] may be calculated by plotting σ(t)/λ(t) as a function of time, and these plots are pictured in FIG. 5B. At small deformations (λ≈10%) these data sets collapse to an apparent plateau at [symbol] E(t) [symbol] ≈100 MPa, although there is more variability in this value for samples that are stretched slowly. After stretching past yield points, all curves display a drop in slope, indicating elastomers have entered a new stress-relaxation regime, which is consistent with scaling predictions in the literature. (See, e.g., X. Hu, J. Zhou, W. F. M. Daniel, M. Vatankhah-Varnoosfaderani, A. V. Dobrynin, S. S. Sheiko, Macromolecules 2017, 50, 652, the disclosure of which is incorporated herein by reference in its entirety).

### 1.3.2. Effect of SP Content

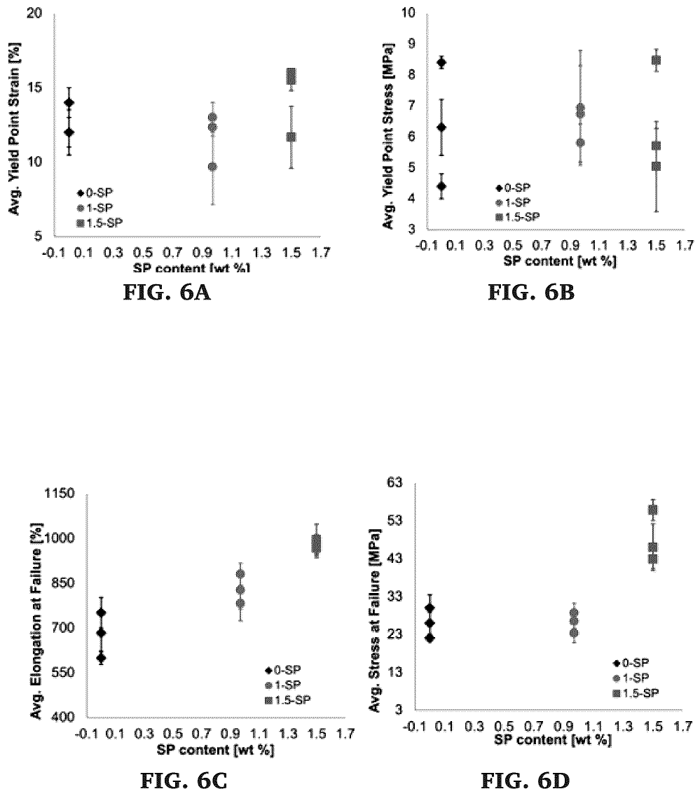

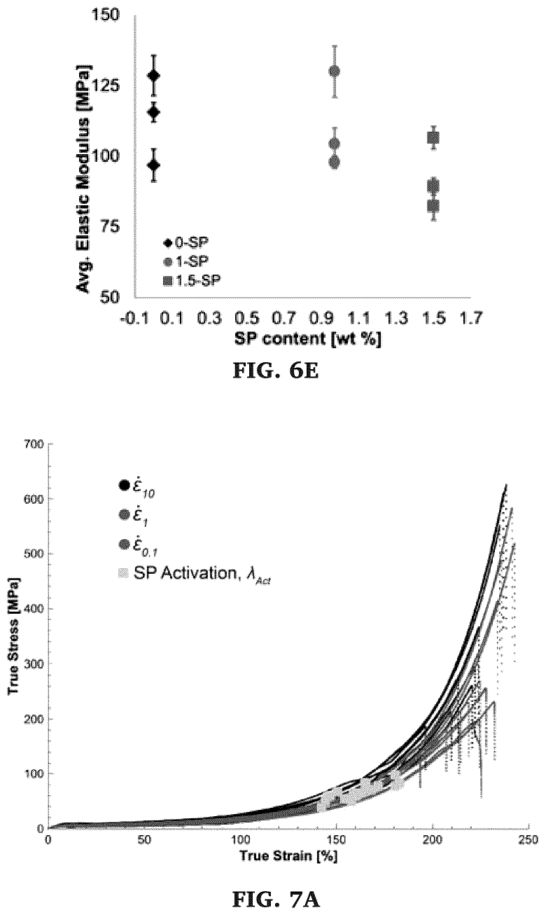

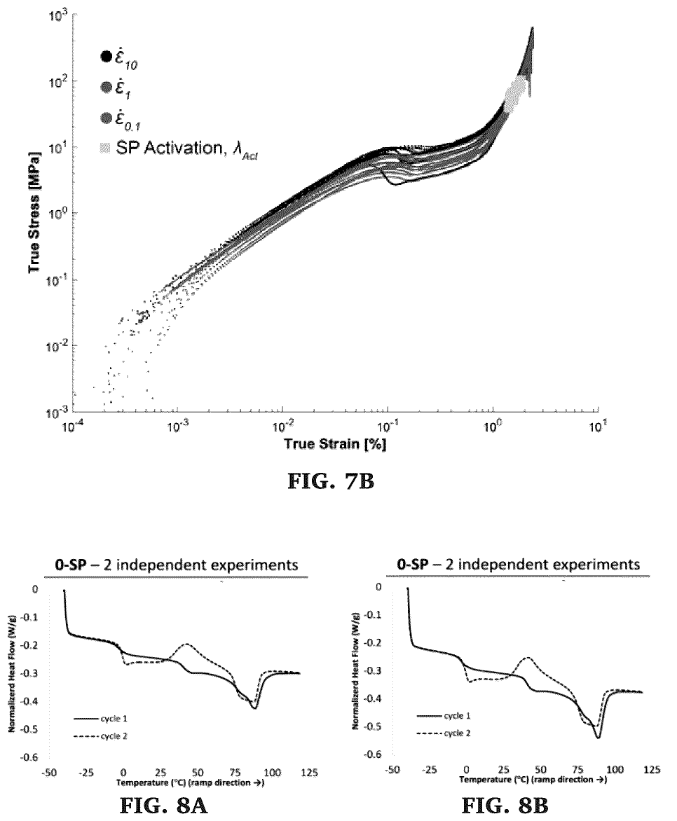

Stress vs. λ curves are pictured in FIG. 5C over the entire deformation range for all elastomers. (See also, FIGS. 6A-E). The corrected true stress (σ_{True}=σ(1+λ)) was calculated to examine the effect of the added SP on material properties. These curves were normalized by dividing by their respective \dot{ε}(s⁻¹), then plotted as a function of time in FIG. 5D. All curves for each \dot{ε} set collapse onto one another, indicating that SP-doped elastomer properties are indistinguishable from those of undoped controls. Similarly, when σ_{True} is plotted as a function of true elongation (λ_{True}=ln (1+λ)), all tensile curves, for all \dot{ε}, collapse onto one another (FIGS. 7A-B). This demonstrates that the SP mechanophore is acting as a true reporter of the mechanical state of the nascent thiol-yne elastomer.

At small deformations, these curves collapse into a straight line, which confirms that within this range, the mechanical properties of the elastomers are independent of the applied \dot{ε}; importantly, this behavior is consistent with other reports of thiol-yne elastomers. (See, e.g., C. A. Bell, J. Yu, I. A. Barker, V. X. Truong, Z. Cao, A. V. Dobrynin, M. L. Becker, A. P. Dove, Angew. Chem. Int. Ed. 2016, 55, 13076, the disclosure of which is incorporated herein by reference in its entirety). As samples are stretched past their yield points, all curves show a corresponding characteristic plateau; at this point, curves of each \dot{ε} set shift horizontally from one another, which suggests that elastomers have entered regimes that are \dot{ε}-dependent. Finally, the slopes increase again as elastomers are stretched to a λ associated with SH.

The molecular behavior that correlates with these macroscopic observables was probed by monitoring the extent of SP activation. All mechanochromic samples underwent a transition from a gradual to an abrupt increase in color, with further strain. The extension at which this transition occurs (λ_{Act}) is defined by the extrapolated intersection of the two color vs. λ regimes; the details of this procedure are discussed in Section 1.7, below. When the points corresponding to λ_{Act} are added to the macroscopic mechanical plots, they are somewhat scattered in the raw data of FIG. 5C but cluster tightly according to \dot{ε} in the scaled plot of FIG. 5D.

### 1.4. Differential Scanning Calorimetry

### 1.4.1. Thermal Properties of Pristine Elastomers

Differential scanning calorimetry (DSC) was performed on a series of samples (5-10 mg) using temperature ramps of 10° C. min⁻¹ (heating and cooling) over the range −20° C. to 140° C. The glass transition temperature (T_{g}), crystallization temperature (T_{c}), Tₘ, and enthalpy of melting (ΔHₘ) were determined from the second heating cycle of DSC, unless otherwise noted. All tests were completed in duplicate, and all results were averaged from 2 independent trials.

Stereoelastomer thermal properties, listed in Table 2 (Appendix), are consistent with those previously reported for 0-SP. (See, e.g., C. A. Bell, J. Yu, I. A. Barker, V. X. Truong, Z. Cao, A. V. Dobrynin, M. L. Becker, A. P. Dove, Angew. Chem. Int. Ed. 2016, 55, 13076, the disclosure of which is incorporated herein by reference in its entirety). These thiol-yne stereoelastomers possess an average T_{g} between of −0.8 and 2.3° C., and all samples displayed crystallization exotherms in their 2^{nd} heating cycles, at T_{c} of 41.6, 38.9, and 40.7° C., for 0-SP, 1-SP, and 1.5-SP, respectively. Additionally, all polymers displayed a distinct Tₘ in both the 1^{st} and 2^{nd} heating cycles, which is also consistent with previous reports. (See, e.g., C. A. Bell, J. Yu, I. A. Barker, V. X. Truong, Z. Cao, A. V. Dobrynin, M. L. Becker, A. P. Dove, Angew. Chem. Int. Ed. 2016, 55, 13076, the disclosure of which is incorporated herein by reference in its entirety). The calculated average normalized ΔHₘ were 14.8, 15.8, and 14.6 J g⁻¹, for 0-SP, 1-SP, and 1.5-SP, respectively.

### 1.4.2. DSC to Confirm Strain Rate-Dependent SIC

It has been reported previously that unstretched thiol-yne polymers of 80% cis content are approximately 22% crystalline; this inherent crystallinity is endowed by ordering of the alkene backbone and is independent of polymer molecular mass. Within these materials, increased crystallinity is correlated with an increased melting peak area in DSC thermograms, which was confirmed with x-ray diffraction. Additionally, previous reports confirmed that, after stretching at 20 mm min⁻¹, crystallinity doubles to more than 50% due to SIC. (See, e.g., C. A. Bell, J. Yu, I. A. Barker, V. X. Truong, Z. Cao, A. V. Dobrynin, M. L. Becker, A. P. Dove, Angew. Chem. Int. Ed. 2016, 55, 13076, the disclosure of which is incorporated herein by reference in its entirety). Because these elastomers exhibit \dot{ε}-dependent SH behavior, it is of interest to confirm the \dot{ε}-dependence of the extent of SIC.

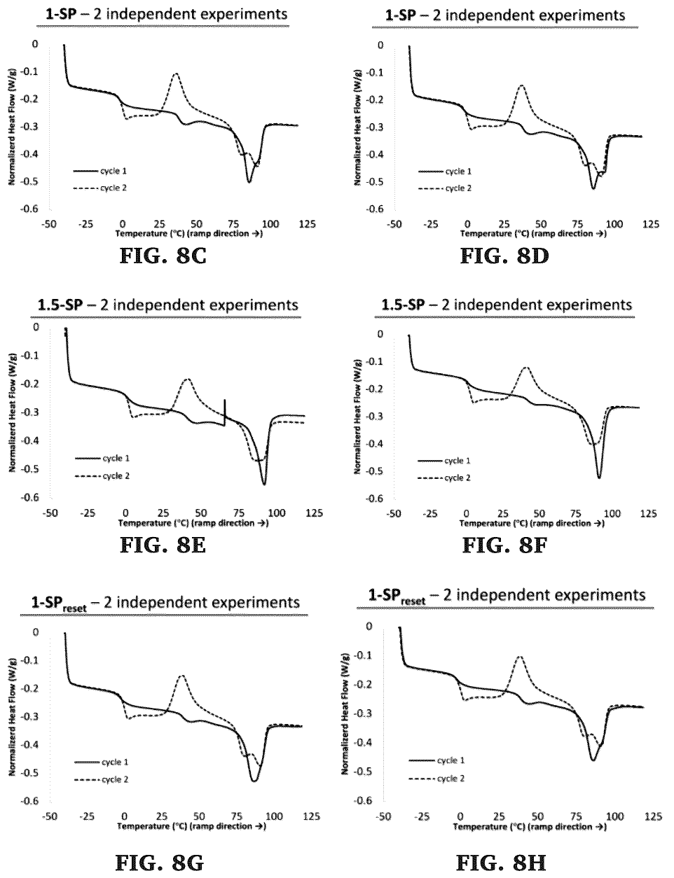

Tensile tests with subsequent DSC analyses were carried out on 0-SP to demonstrate the extent of SIC for the different \dot{ε}, quantified by the increase in enthalpy. Several measures were taken to ensure that all samples possessed similar thermal histories. First, all tensile samples were cut from the same film, which had been melt-pressed as described in the previous sections. Samples strained at \dot{ε}₁₀ and \dot{ε}₁ were stretched until failure, then immediately stored at −78° C. before DSC analysis. Films were handled only with forceps, as opposed to fingers, which prevented any SIC melting from body heat. An unstretched control film was stored under the exact same conditions, and DSC was performed on the same day. Samples of 5-10 mg were cut using a razor blade, and samples for stretched films were always taken from the fractured ends. All samples were heated and cooled for two DSC cycles under the method mentioned above, and this experiment was repeated for 2 independent sets. (See, FIGS. 8A-F) Data are reported as the average percent increase in ΔHₘ, relative to the respective control values.

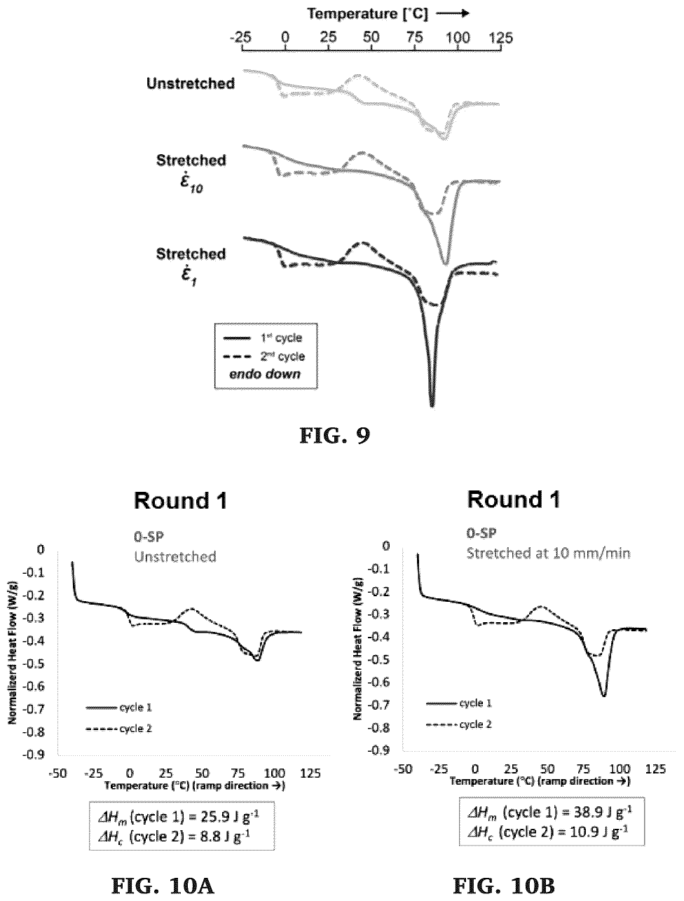

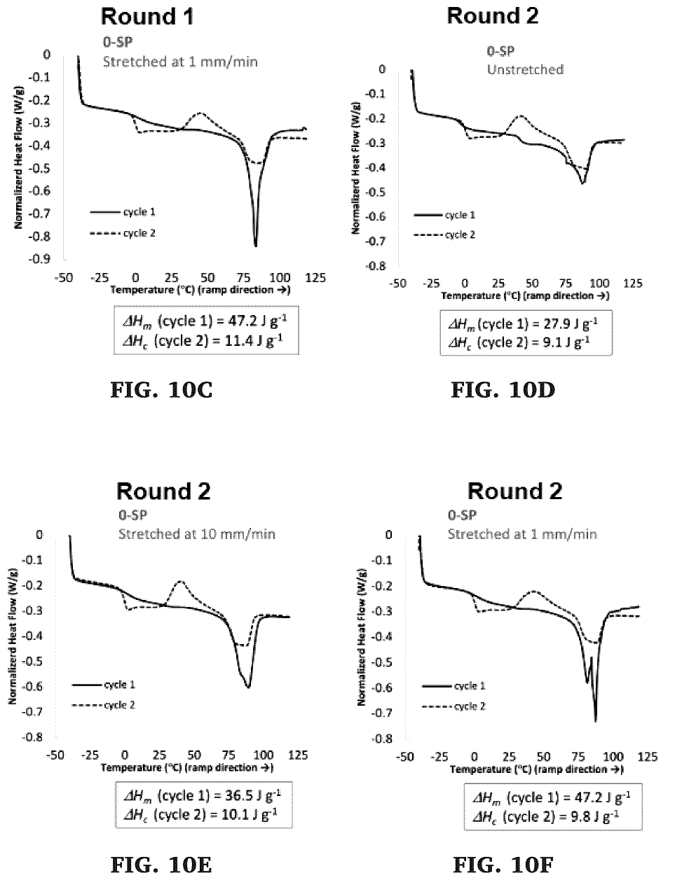

These results are listed in Table 6 (Appendix), and heating curves from one experimental set are pictured in FIG. 9 (See also, FIGS. 8A-H, 10A-F). As expected, in the 1^{st} cycle, stretched samples displayed larger Tₘ peak areas when compared to the unstretched control. For \dot{ε}₁₀-tested sample, there is a 48% average increase in ΔHₘ (cycle 1); when tested at \dot{ε}₁, ΔHₘ increased by 86%, relative to unstretched controls. Importantly, this \dot{ε}-dependence is not observed in the 2^{nd} cycle data; there is no significant increase from control values, indicating that any strain-induced crystallites had melted. Some samples depict small shifts in Tₘ to lower temperatures after stretching, however further experiments must be conducted before making any quantitative conclusions about their significance. These results confirm that the extent of SIC is indeed dependent on the applied \dot{ε}: SIC is observed within the 1^{st} cycle, then the 2^{nd} cycle is reflective of the polymer's intrinsic (non-SIC) crystallinity.

### 1.5. Image Analysis

Pixel data from digital images captured during each tensile test served as the raw data to quantify SP activation and subsequent relaxation. These images were captured in regular intervals using an intervalometer, such that each image represented either 1 mm or 2 mm displacement by the crosshead beam; the corresponding λ associated with each image was calculated from this displacement interval and the original sample gauge lengths.

Image processing was done using Fiji (Image J), according to methods described previously in the literature. (See, e.g., G. R. Gossweiler, G. B. Hewage, G. Soriano, Q. Wang, G. W. Welshofer, X. Zhao, S. L. Craig, ACS Macro Letters 2014, 3, 216 and M. H. Barbee, K. Mondal, J. Z. Deng, V. Bharambe, T. V. Neumann, J. J. Adams, N. Boechler, M. D. Dickey, S. L. Craig, ACS Appl. Mater. Interfaces 2018, 10, 29918, the disclosures of which are incorporated herein by reference in their entirety). Images within each data set were split into their red, green, and blue color channels. The mean pixel intensities of each channel, R, G, and B, were measured within a small rectangular region of interest (ROI). This process was completed for all images within each tensile test data set, taking extra care to ensure the ROI was always within the center of the tensile sample with no overlap at the sample edge.

The chromatic change of the blue channel was used to measure SP activation, and it was calculated as described in a previous publication. (See, Y. Chen, C. J. Yeh, Y. Qi, R. Long, C. Creton, Sci. Adv. 2020, 6, eaaz5093, the disclosure of which is incorporated herein in its entirety). First, the total chromaticity, S, was calculated for each image as the sum of R, G, and B. Next, the chromaticity of the blue channel was defined as the ratio B S⁻¹, which describes how the blue signal intensity compares to the other color channels. Finally, B S⁻¹ values for each set of images were normalized to values at λ=0%, giving the chromatic change of the blue channel, ΔS_{blue}, as shown in Equation (2):

$$Δ {S}_{blue} = \frac{B \cdot {S}^{- 1}}{{( B \cdot {S}^{- 1} )}_{λ = 0}} \qquad ( 2 )$$

### 1.6. Chromaticity Results

For all samples, including 0-SP, R, G, and B increase dramatically for the first 50-80% displacement, which is attributed to a shadow effect from the top gripper on the Instron (FIG. 5A). As the gripper moves out of the camera field of view, more photons can reach the camera lens, increasing mean pixel intensities for all color channels. The corresponding ΔS_{blue} behavior within this region is variable, and, in some cases, ΔS_{blue} decreases initially from its value at λ=0%. For this reason, subsequent data fittings were completed only for images obtained after gripper shadow effects had dissipated, as confirmed with image histograms in Adobe Lightroom.

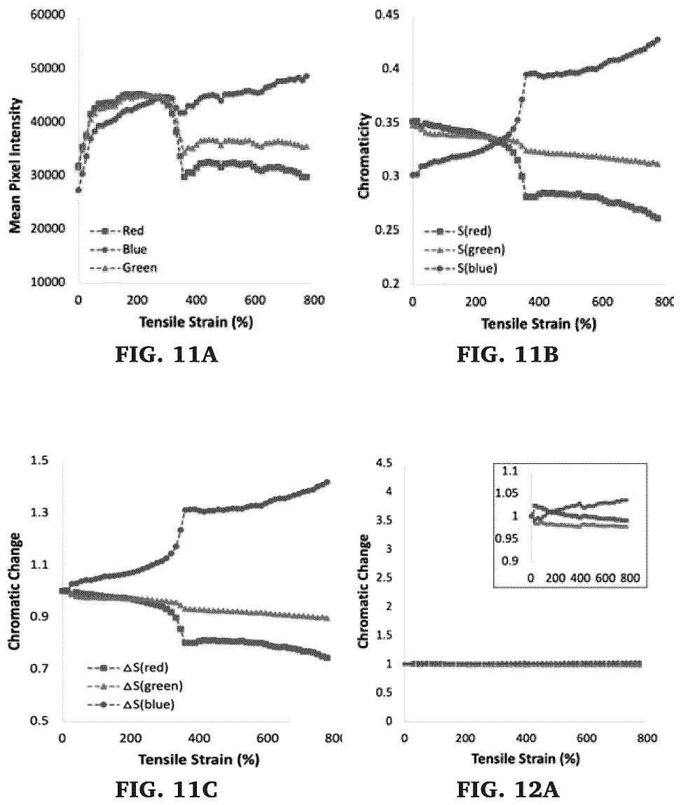

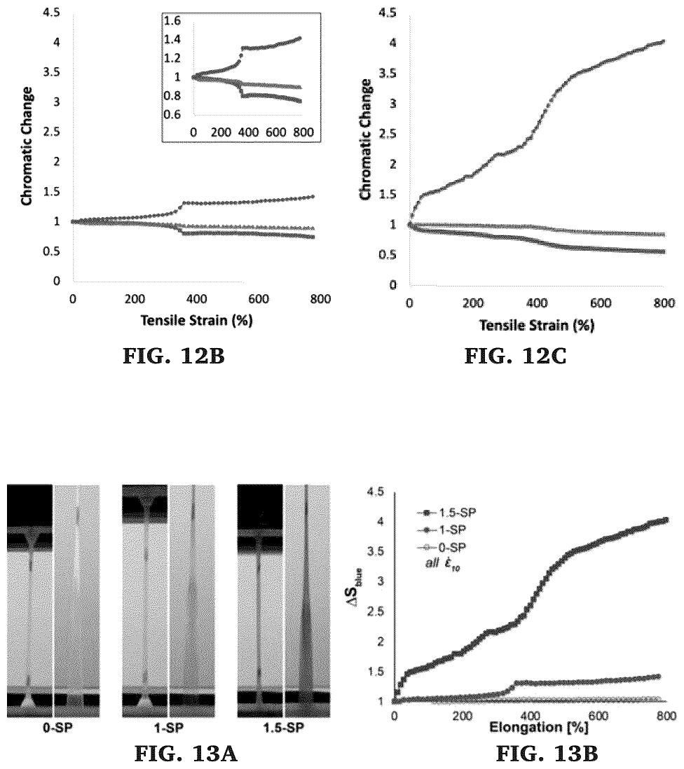

At sufficiently high strains, polymers 1-SP and 1.5-SP exhibit a blue color that is visible to the naked eye. The color change is observed only after polymers have entered SH, and this behavior is consistent with literature precedent. (See e.g., D. A. Davis, A. Hamilton, J. Yang, L. D. Cremar, D. Van Gough, S. L. Potisek, M. T. Ong, P. V. Braun, T. J. Martinez, S. R. White, J. S. Moore, N. R. Sottos, Nature 2009, 459, 68, G. R. Gossweiler, G. B. Hewage, G. Soriano, Q. Wang, G. W. Welshofer, X. Zhao, S. L. Craig, ACS Macro Letters 2014, 3, 216, and Y. Lin, M. H. Barbee, C. C. Chang, S. L. Craig, J. Am. Chem. Soc. 2018, 140, 15969, the disclosures of which are incorporated herein by reference in their entirety). Mechanochromism is manifested within pixel data as a dramatic increase in ΔS_{blue}. See, FIGS. 11A-B, 12A-C), At the point of failure, ΔS_{blue} values for 1-SP and 1.5-SP have increased by approximately 50-400% of their initial values, depending on the applied \dot{ε}. In contrast, the maximum ΔS_{blue} for non-mechanochromic 0-SP never increases by more than 5-10% of the original pixel intensities (FIGS. 13A-B). As a result, any observed increase in ΔS_{blue} beyond this 5-10% threshold is attributed to SP activation, rather than deviations in lighting conditions or sample thickness.

Typically, ΔS_{blue} curves for thiol-yne polymers display multiple regimes, and, when plotted as a function of λ, these regimes correlate with the corresponding tensile data. Importantly, this multi-regime behavior differs somewhat from other reports of SP activation. In prior reports, ΔS_{blue} remains mostly constant until polymers reach SH, increases linearly as SP is activated, and finally, may plateau just before material failure. (See, e.g., D. A. Davis, A. Hamilton, J. Yang, L. D. Cremar, D. Van Gough, S. L. Potisek, M. T. Ong, P. V. Braun, T. J. Martinez, S. R. White, J. S. Moore, N. R. Sottos, Nature 2009, 459, 68; G. R. Gossweiler, G. B. Hewage, G. Soriano, Q. Wang, G. W. Welshofer, X. Zhao, S. L. Craig, ACS Macro Letters 2014, 3, 216; Y. Chen, C. J. Yeh, Y. Qi, R. Long, C. Creton, Sci. Adv. 2020, 6, eaaz5093; and Y. Lin, M. H. Barbee, C. C. Chang, S. L. Craig, J. Am. Chem. Soc. 2018, 140, 15969, the disclosures of which are incorporated herein by reference in their entirety). In contrast, ΔS_{blue} curves for 1-SP and 1.5-SP display additional regimes, as evidenced in the multiple inflections of ΔS_{blue} curves. Of particular interest to us is the onset of rapid increase in mechanochromism, where the population of activated chains begins to grow abruptly. We denote this transition point as λ_{Act} and its corresponding tensile stress as s_{Act}, and the details of these calculations are discussed in Section 1.7.

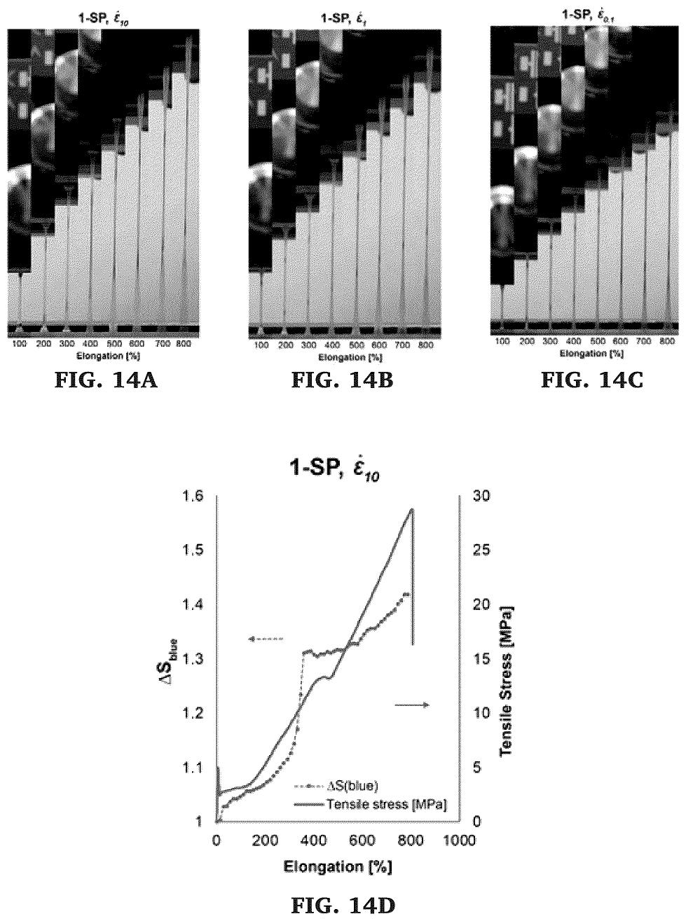

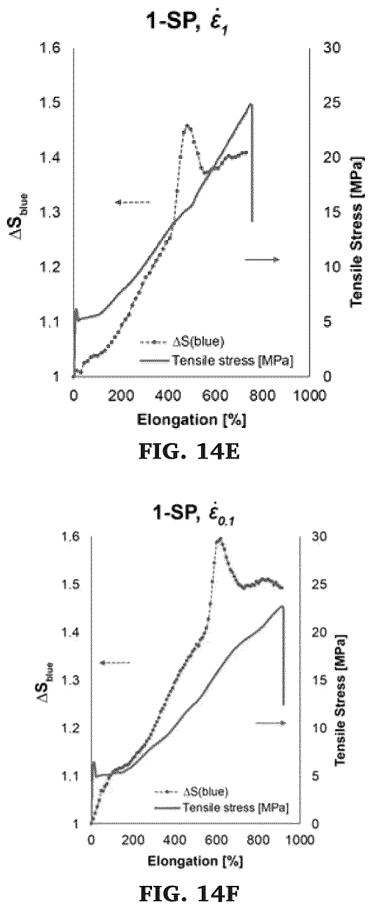

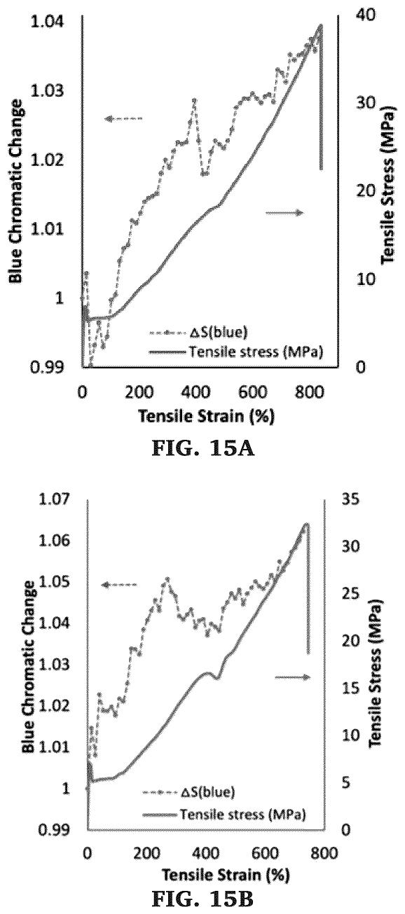

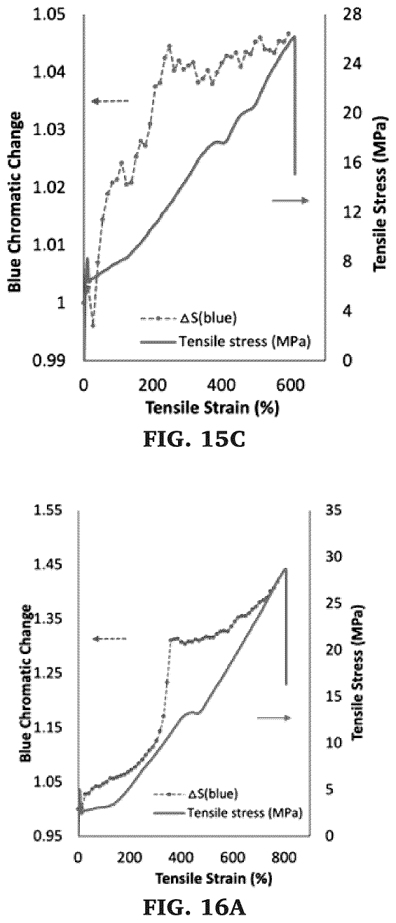

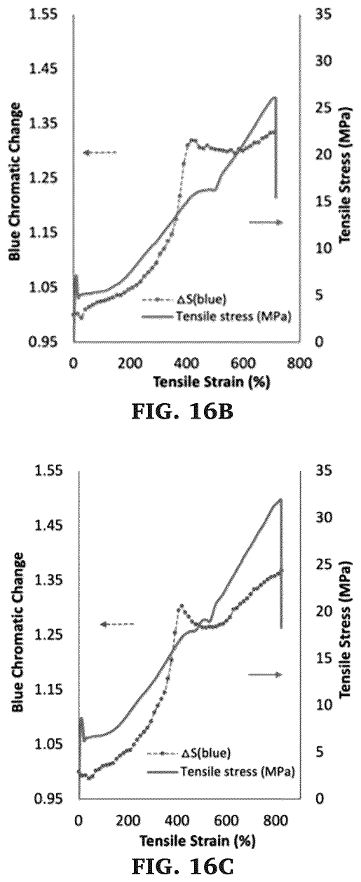

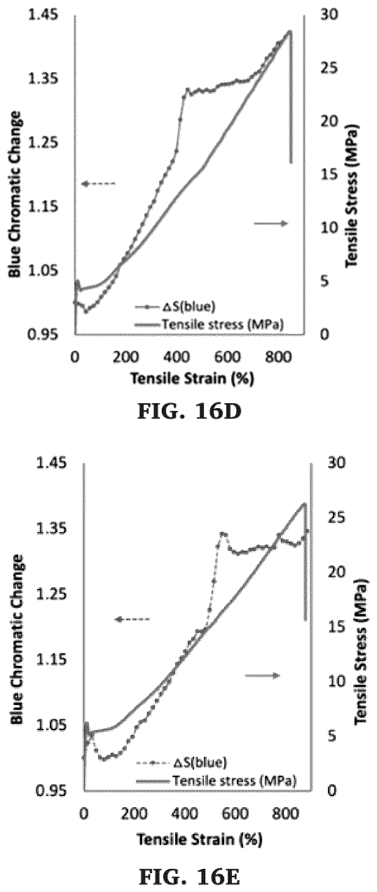

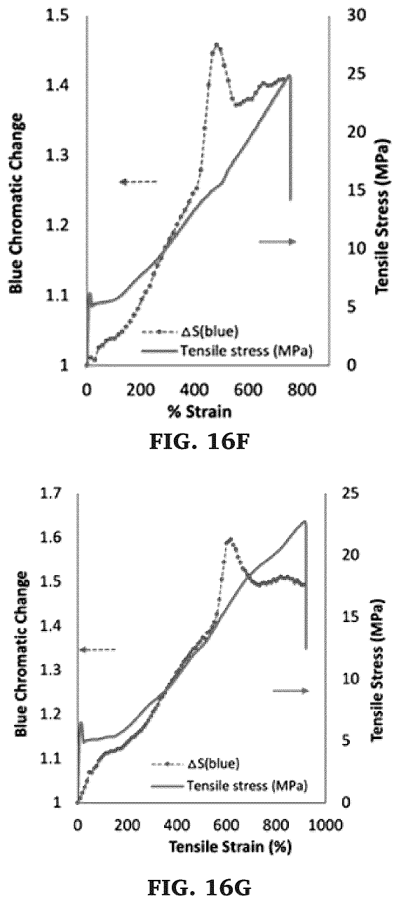

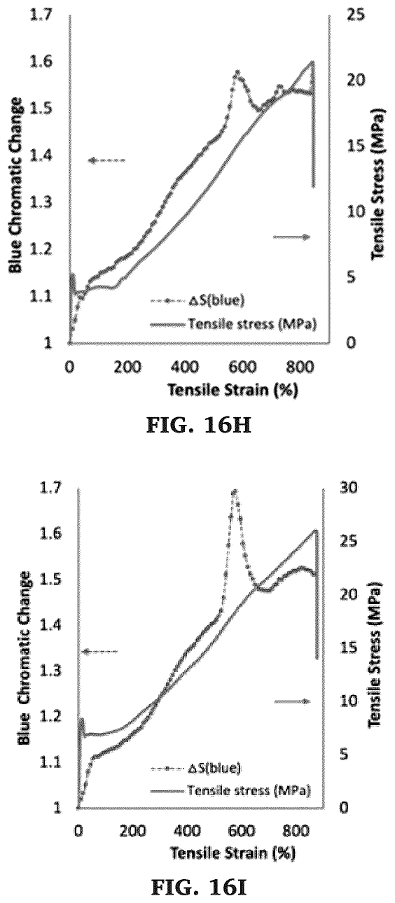

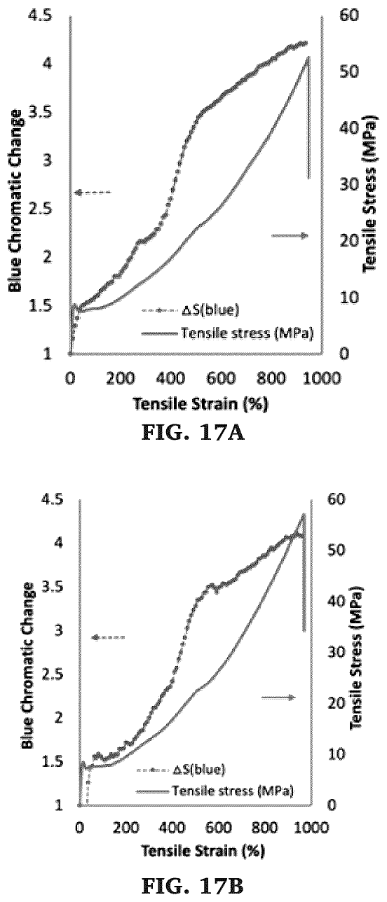

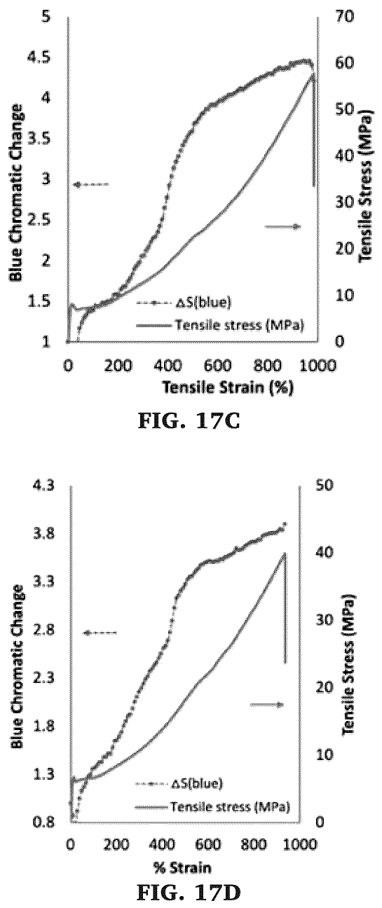

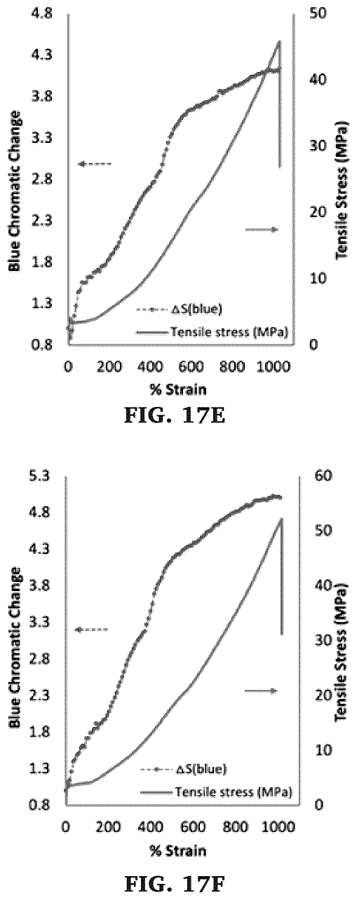

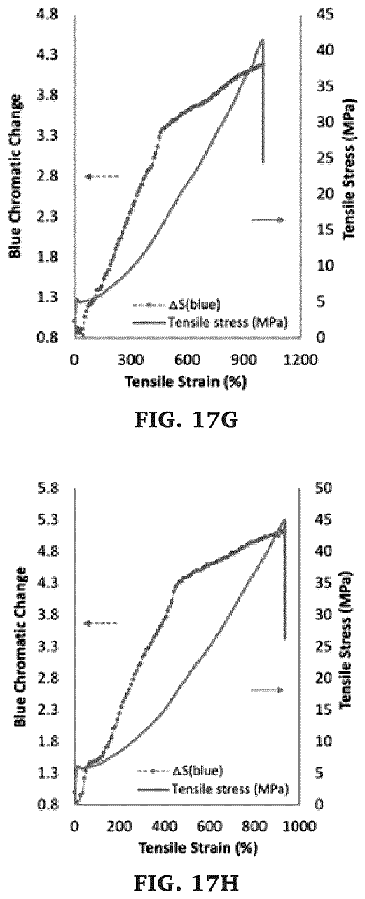

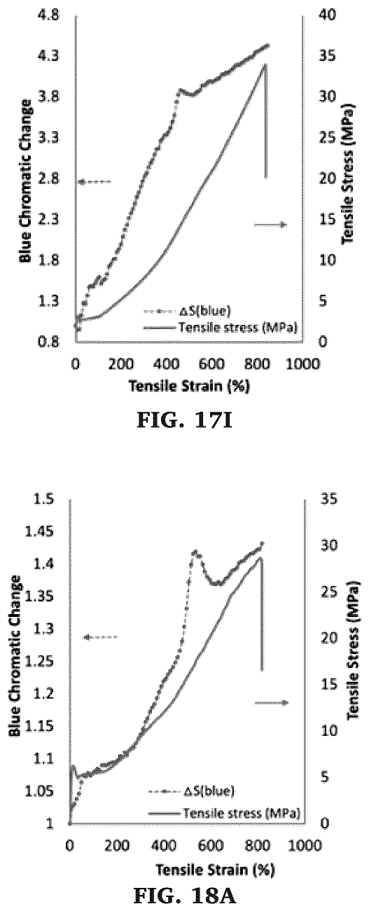

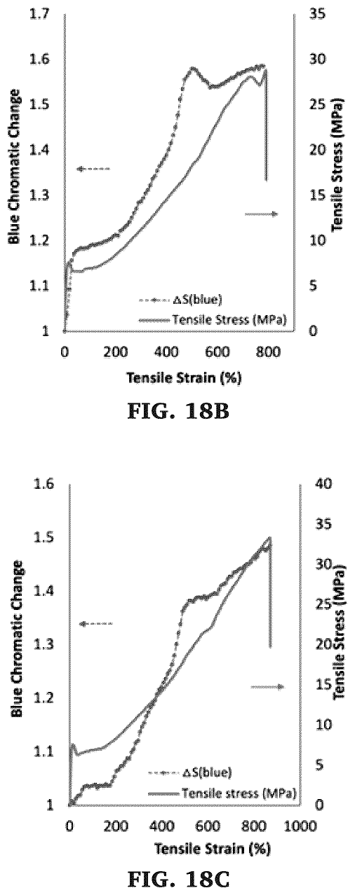

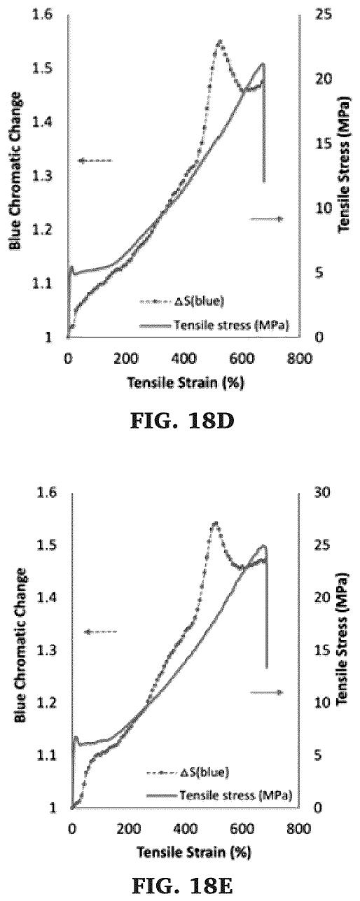

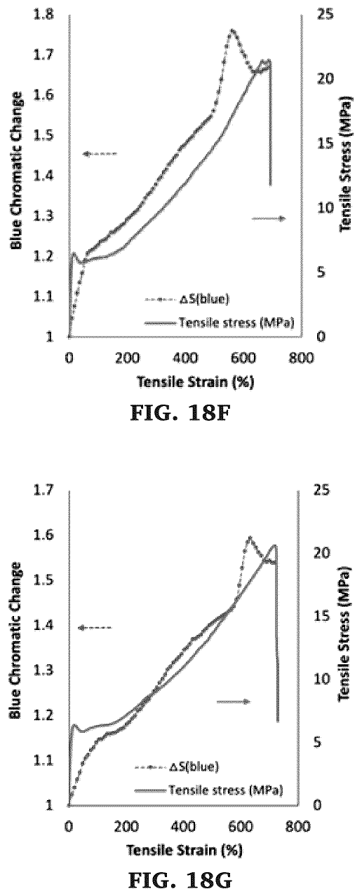

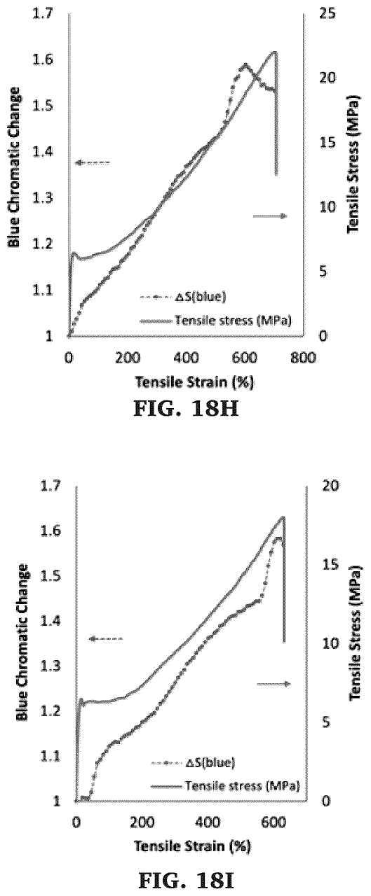

Example ΔS_{blue} and tensile data have been plotted together in FIGS. 14A-F for 1-SP at each \dot{ε}; overlays of data from all triplicate experiments are pictured in FIGS. 15-18. ΔS_{blue} begins to increase linearly at the start of tensile region SH, which usually occurs around λ=140-200%. In some cases, the first ΔS_{blue} regime occurs before SP activation is obvious to the naked eye (see, e.g., FIGS. 14A-C); one example can be seen in FIG. 14B in the image obtained at λ=200%. As polymers are stretched further within early SH, the absolute slope of the ΔS_{blue} vs. λ plot increases dramatically, and images confirm that blue color becomes much more intense. (see, e.g., FIGS. 14D-F). Subsequently, the absolute slope of ΔS_{blue} decreases as polymers transition through later SH tensile regimes; usually this point is marked by a brief period of strain-softening in the tensile data. Finally, just prior to failure, the ΔS_{blue} slope increases again to levels similar to those observed at the beginning of SH. The overall increase in color, and thus ΔS_{blue}, is dependent on the applied \dot{ε}; elastomers stretched slowly become more intensely blue, which is also consistent with results reported in previous publications. (See, e.g., Y. Chen, C. J. Yeh, Y. Qi, R. Long, C. Creton, Sci. Adv. 2020, 6, eaaz5093; M. H. Barbee, T. Kouznetsova, S. L. Barrett, G. R. Gossweiler, Y. Lin, S. K. Rastogi, W. J. Brittain, S. L. Craig, J. Am. Chem. Soc. 2018, 140, 12746; and Y. Lin, Y. Lin, M. H. Barbee, C. C. Chang, S. L. Craig, J. Am. Chem. Soc. 2018, 140, 15969, the disclosures of which are incorporated herein by reference in their entirety).

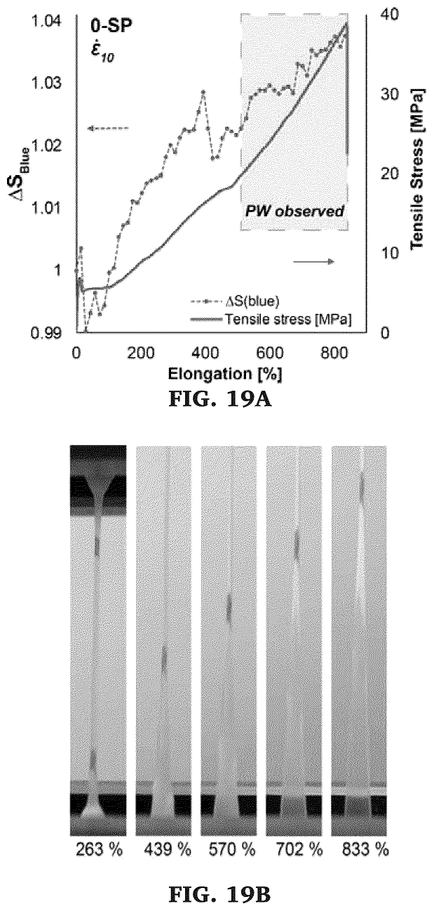

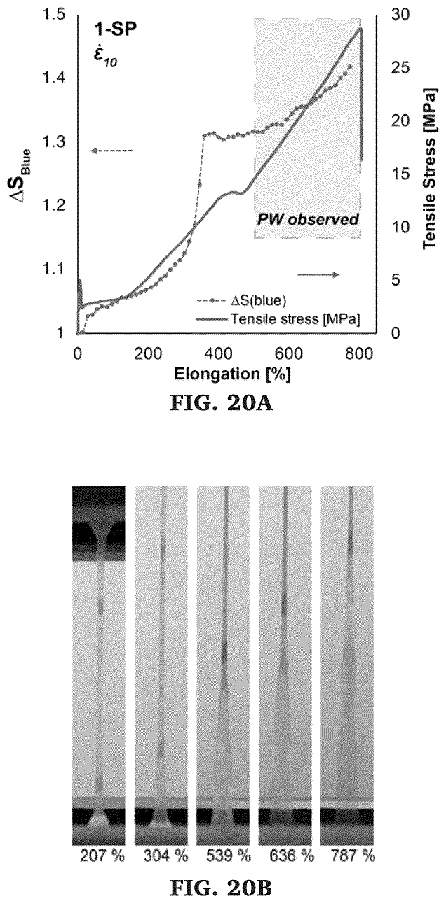

All elastomers exhibit diffraction-based polymer whitening (PW) effects, and these effects also correlate with SH and SIC. PW is marked by an increased opacity that is observed easily by naked eye, and this effect is attributed to underlying changes in morphology. (See, Y. Zhang, P. Y. Ben Jar, S. Xue, L. Li, Journal of Materials Science 2018, 54, 62, the disclosure of which is incorporated herein by reference in its entirety). Due to this increase in scattering, PW effects are also captured within ΔS_{blue} data (FIGS. 19A-B, 20A-B). For these stereoelastomers, data and images for 0-SP most readily illustrate these connections, but it is important to note that these effects are also observed for 1-SP and 1.5-SP. Although 0-SP is not mechanochromic, there are still 2 observable regimes within the noisy ΔS_{blue} curves (FIG. 19A-B), and these regimes correlate with tensile SH regimes. As polymers are stretched through the beginning of SH, ΔS_{blue} increases, most likely because of a decrease in sample thickness. Images of 0-SP captured within early SH (λ≈200-400%) also confirm that samples are still moderately translucent. When samples reach elongations associated with later SH (λ≈550%), there is a marked inflection in ΔS_{blue}, which marks the start of PW; at this point, all materials become noticeably opaque. When comparing to mechanochromic 1-SP, as shown in FIGS. 20A-B, it becomes clear that PW also correlates with multi-regime mechanochromism.

### 1.7. Linear Extrapolation to Define λ_{Act}

In all mechanochromic samples, SP activation is slow and subtle at first, then becomes rapid and intense. The transition from slow to abrupt is indicative of a change in polymer chain behavior that occurs at some critical extension. We name this point of transitional mechanochromic behavior λ_{Act}, and it is defined as this inflection point, which is calculated by the intersection of linear extrapolations of ΔS_{blue} vs. λ from each of these regimes.

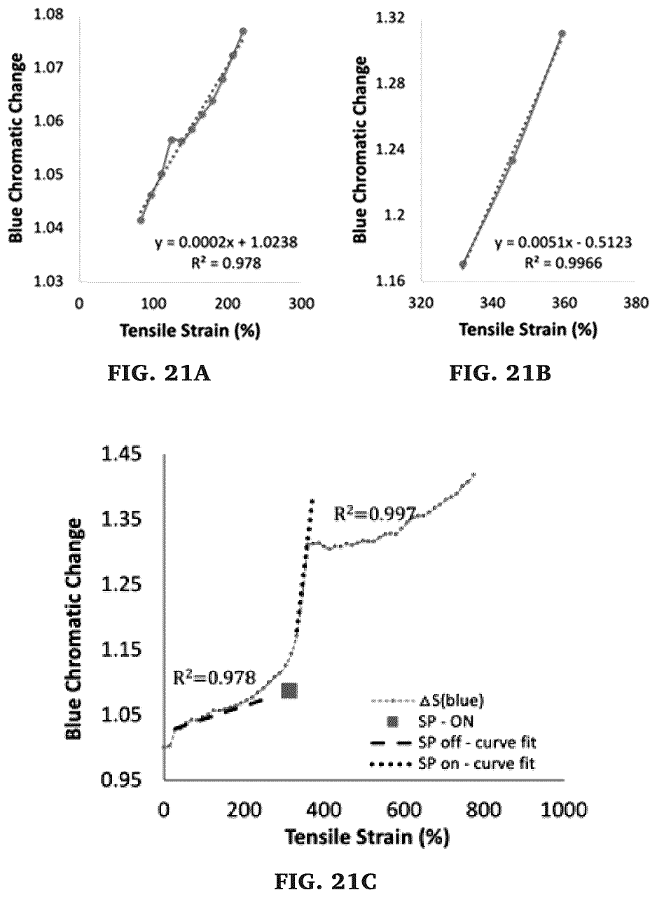

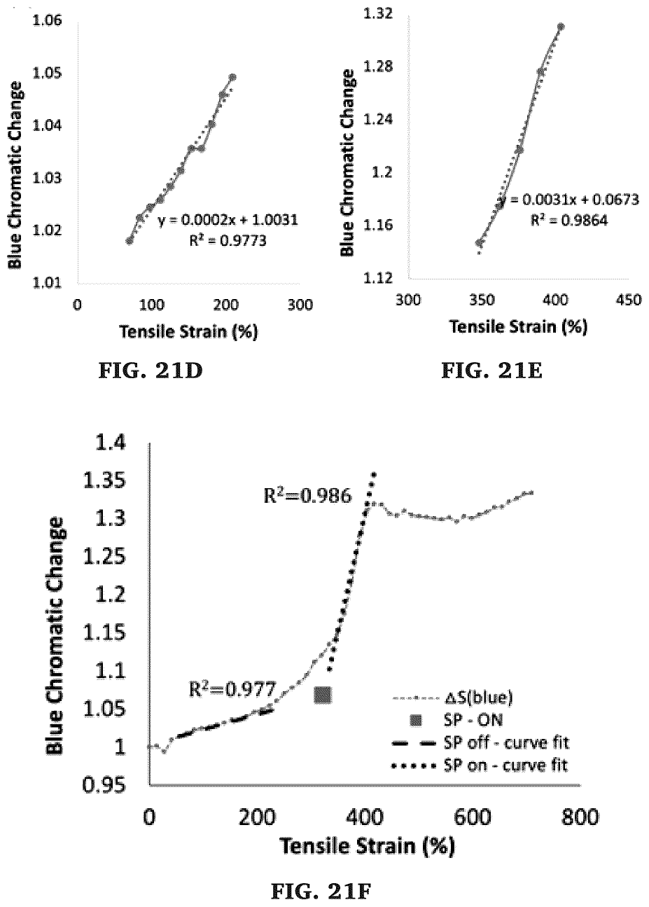

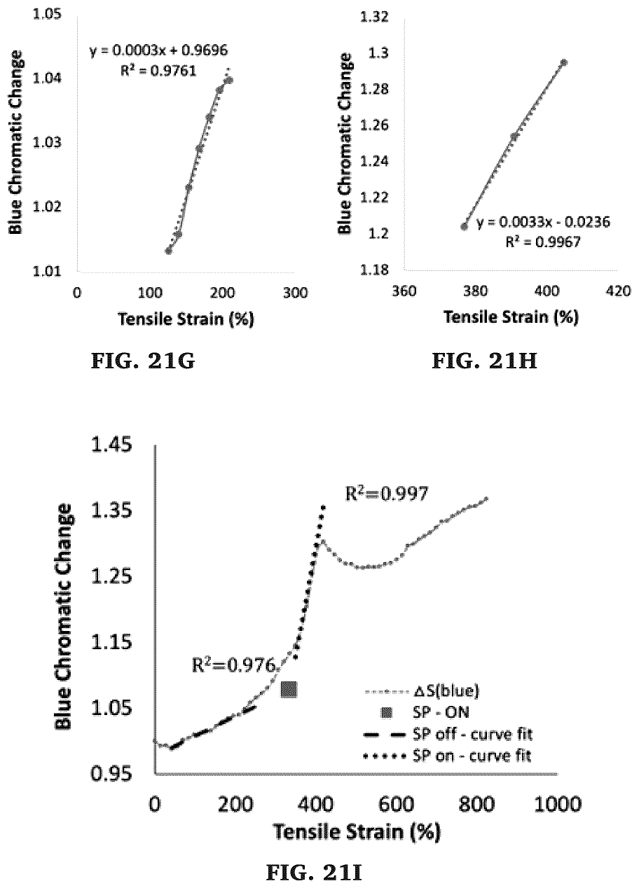

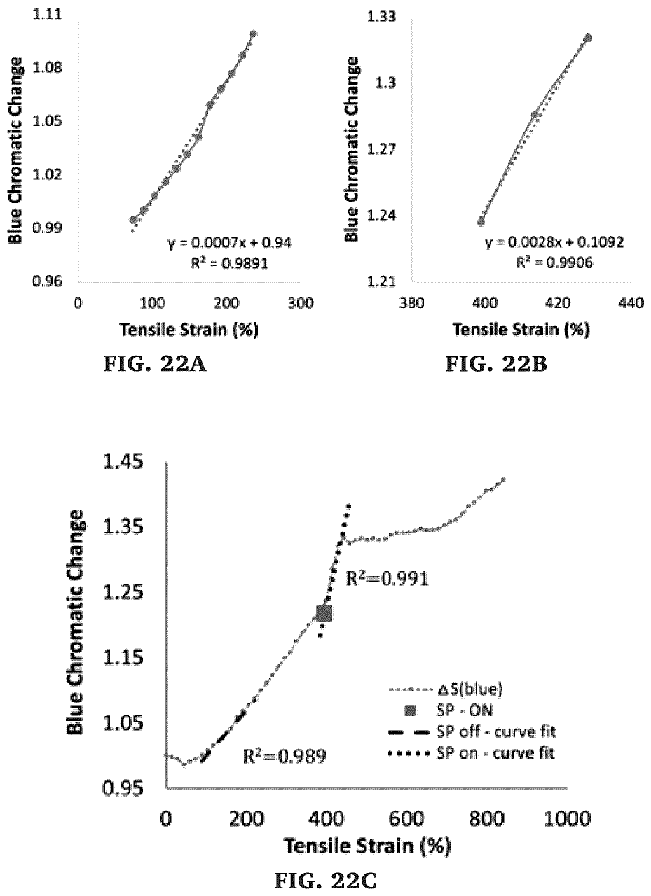

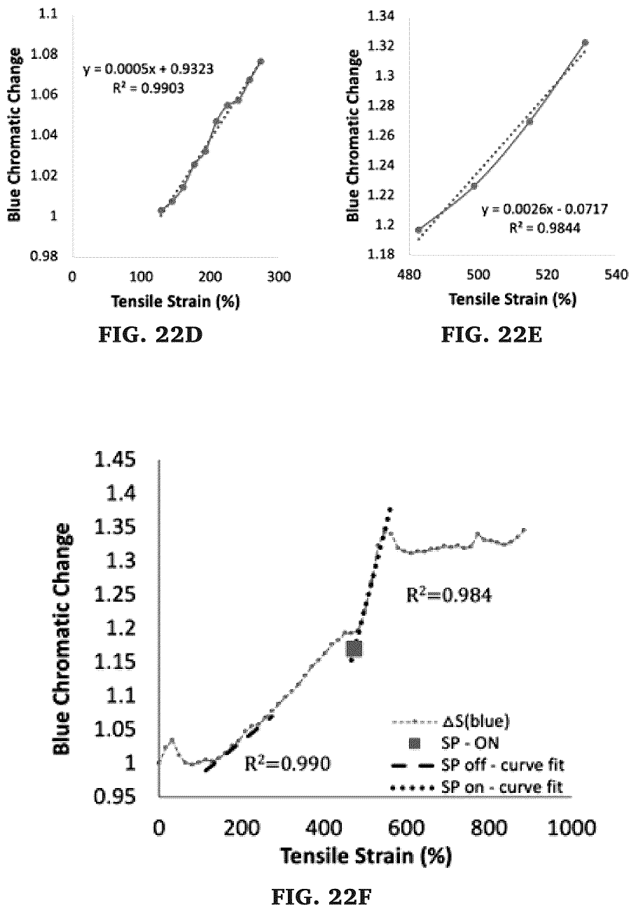

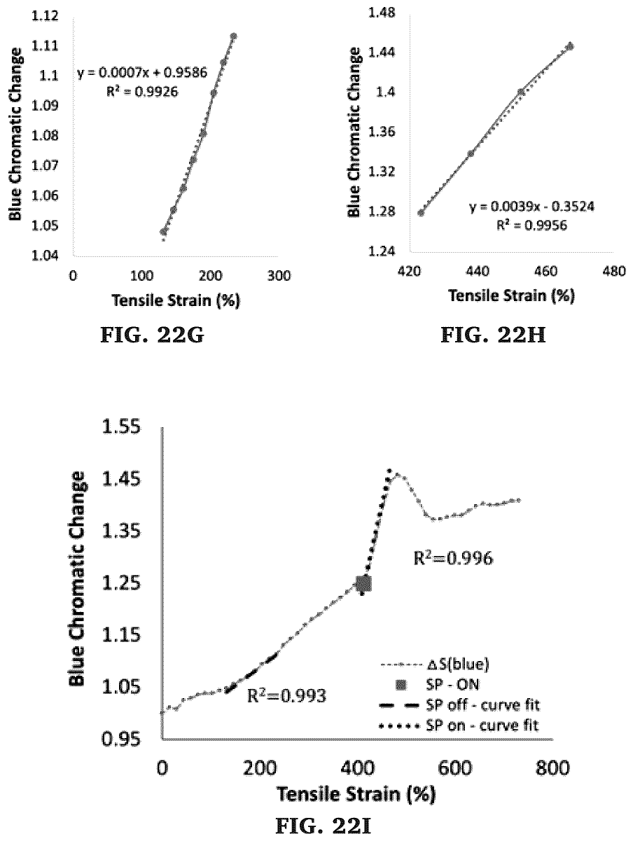

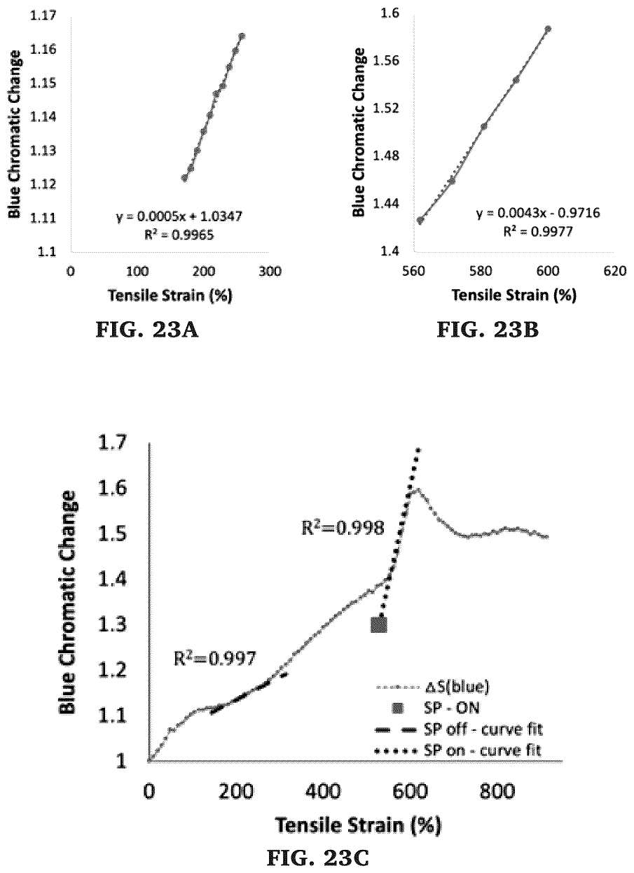

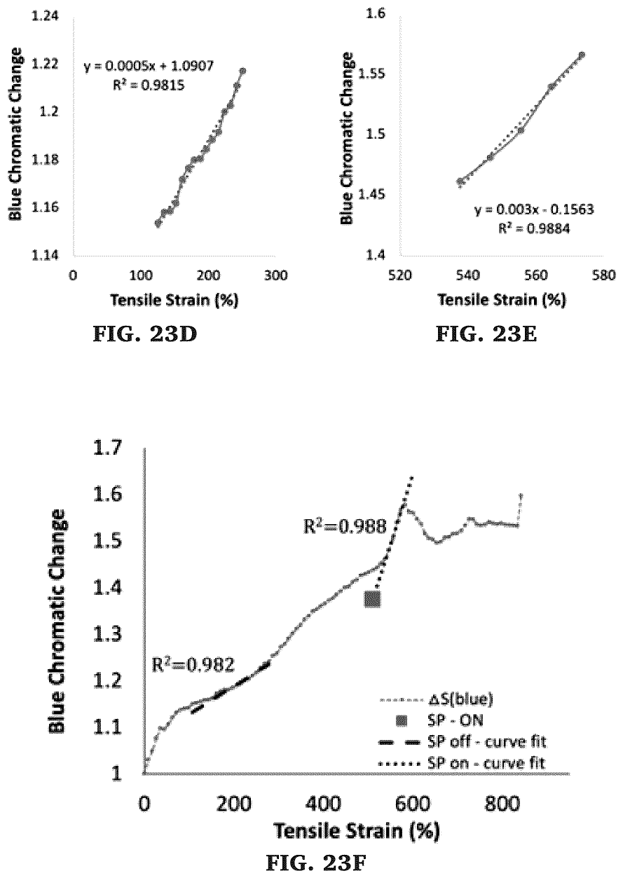

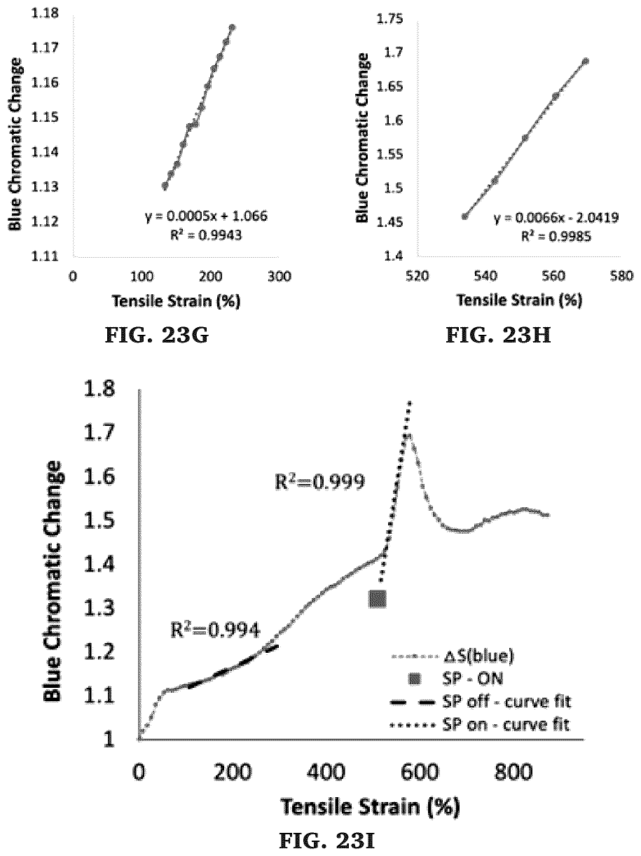

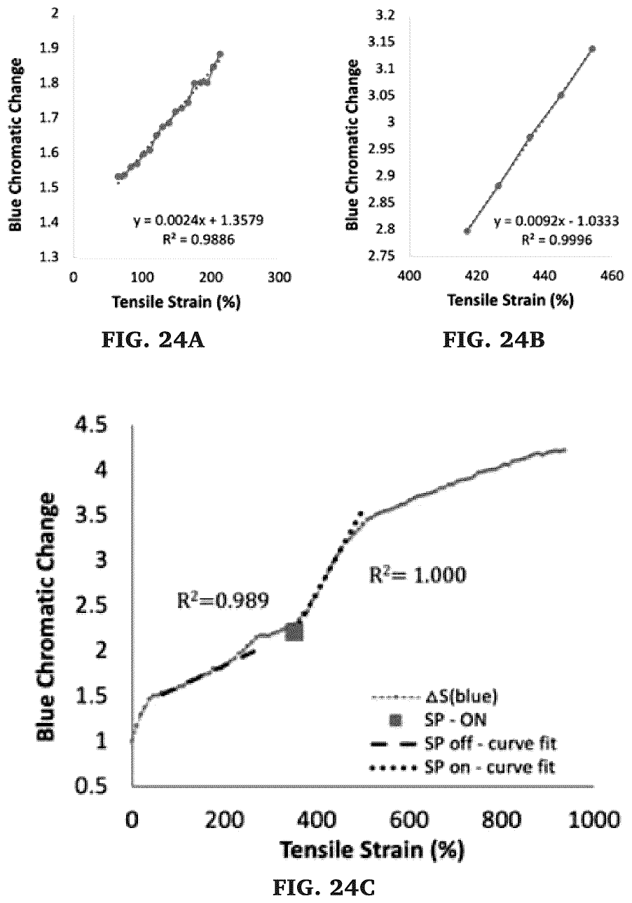

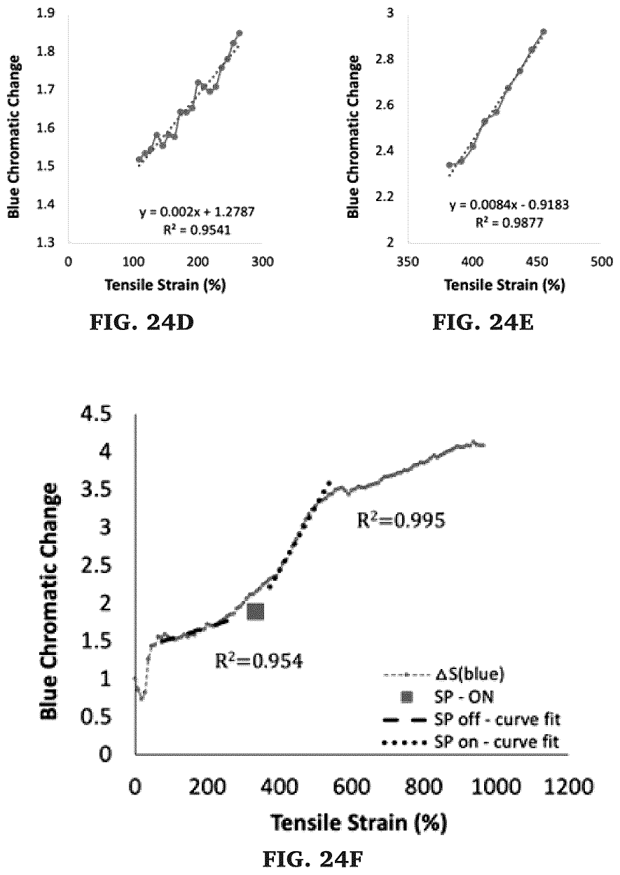

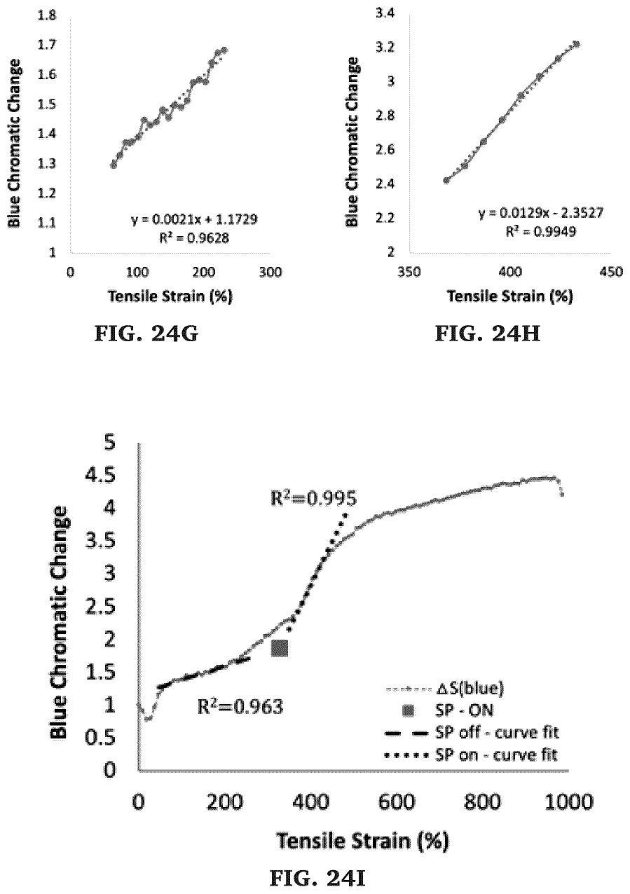

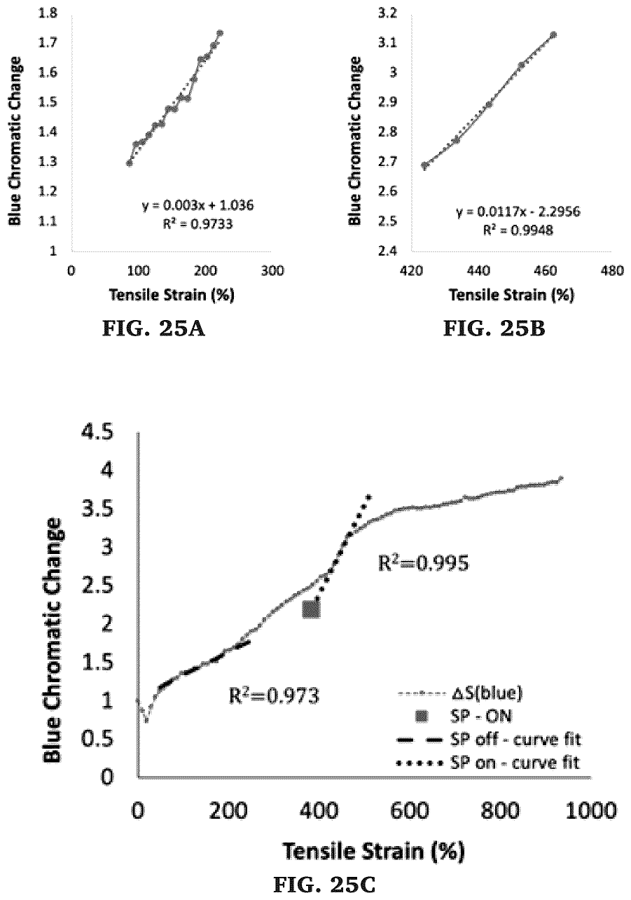

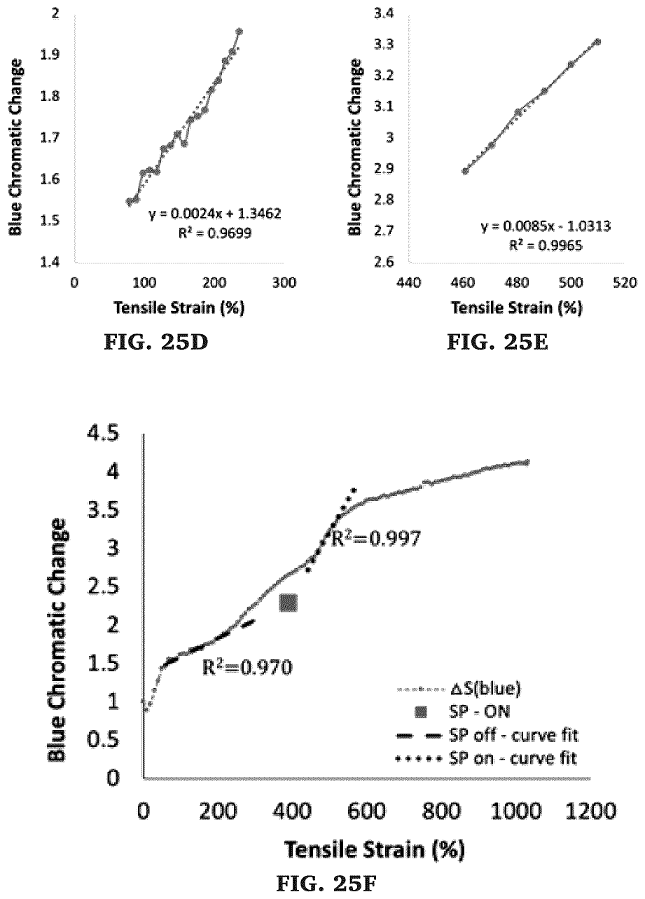

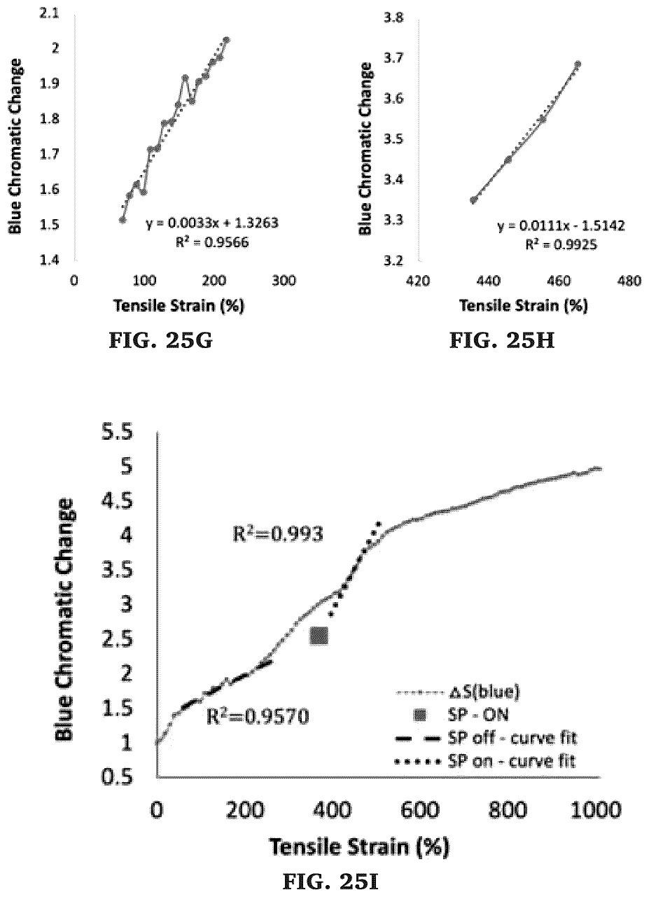

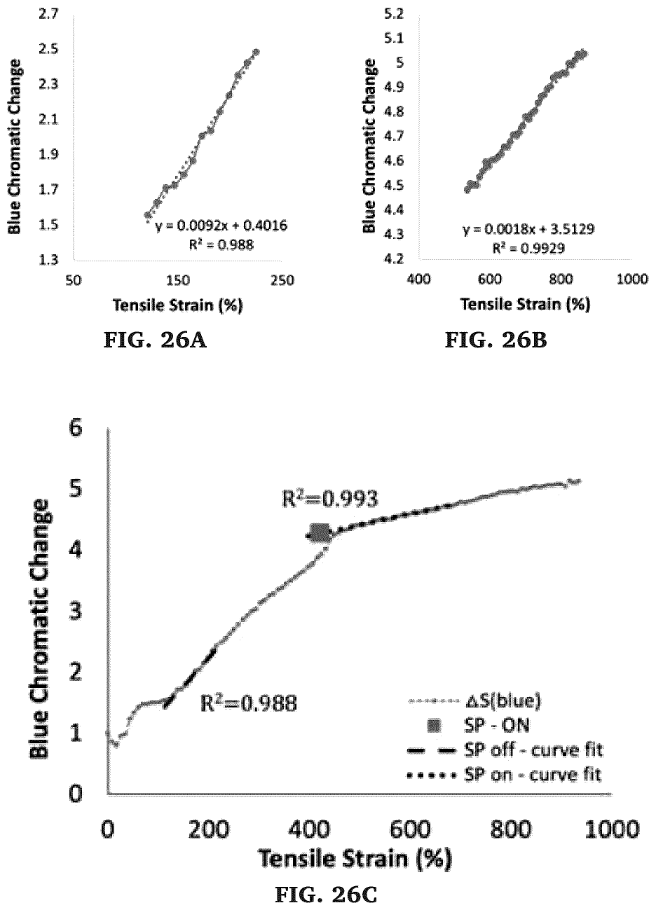

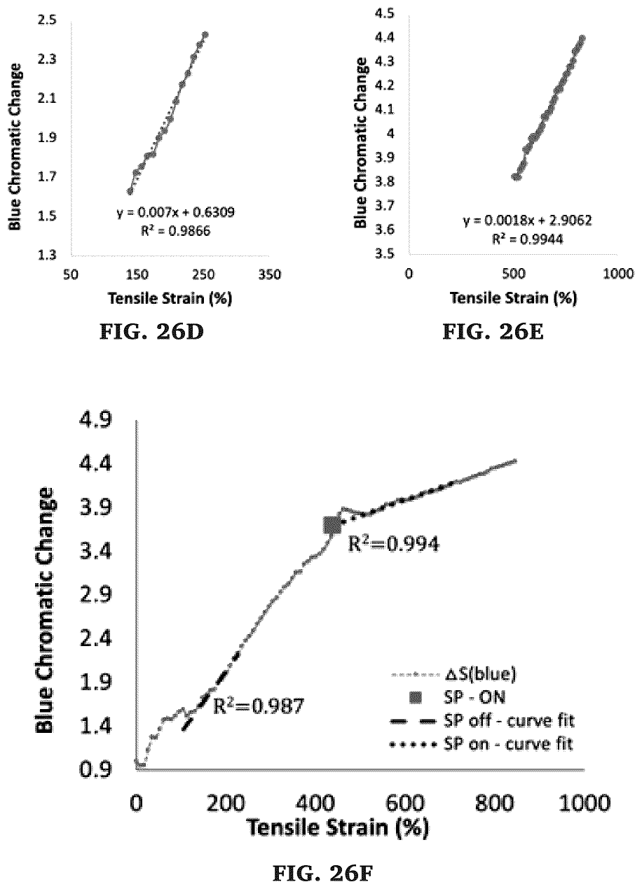

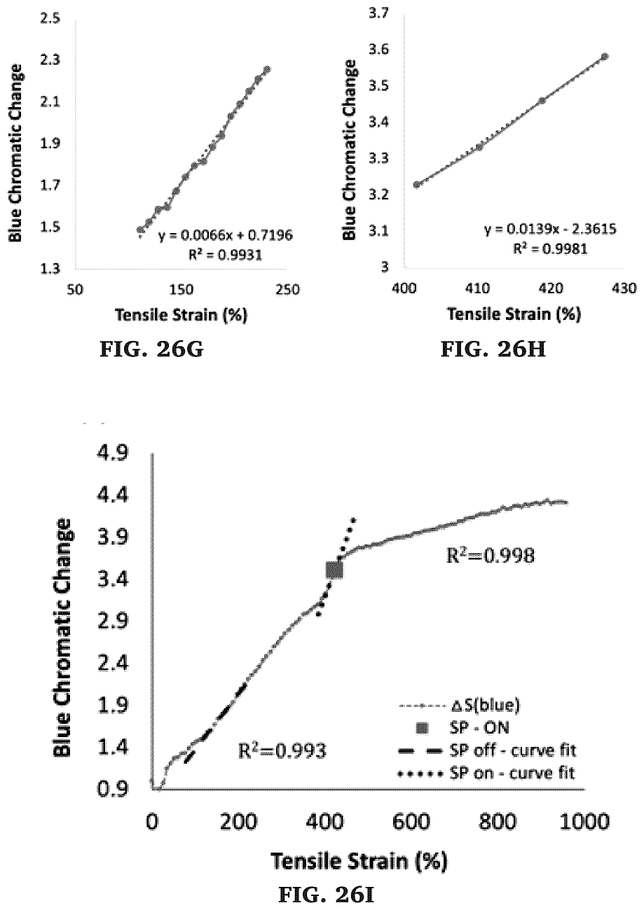

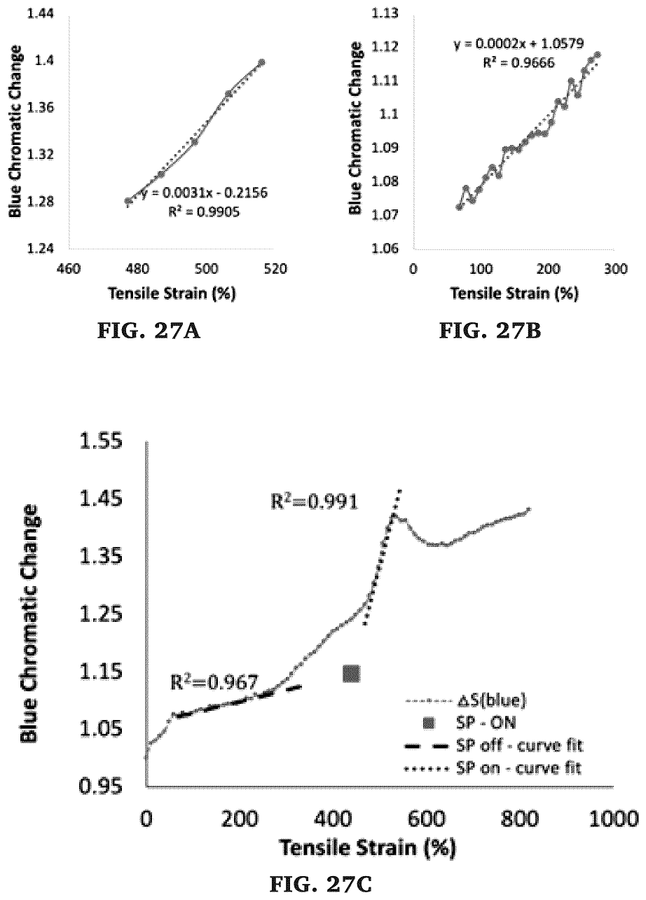

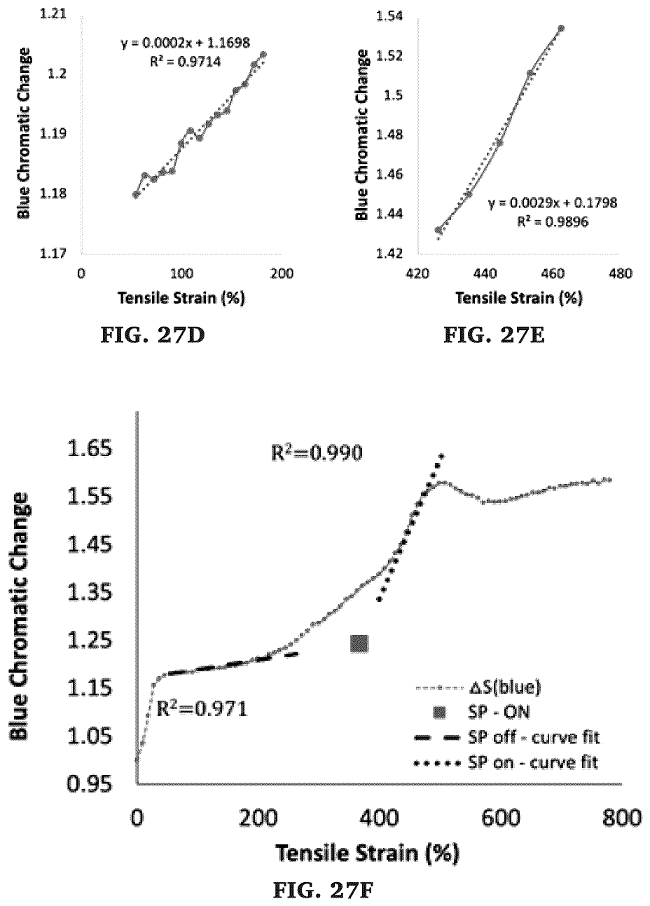

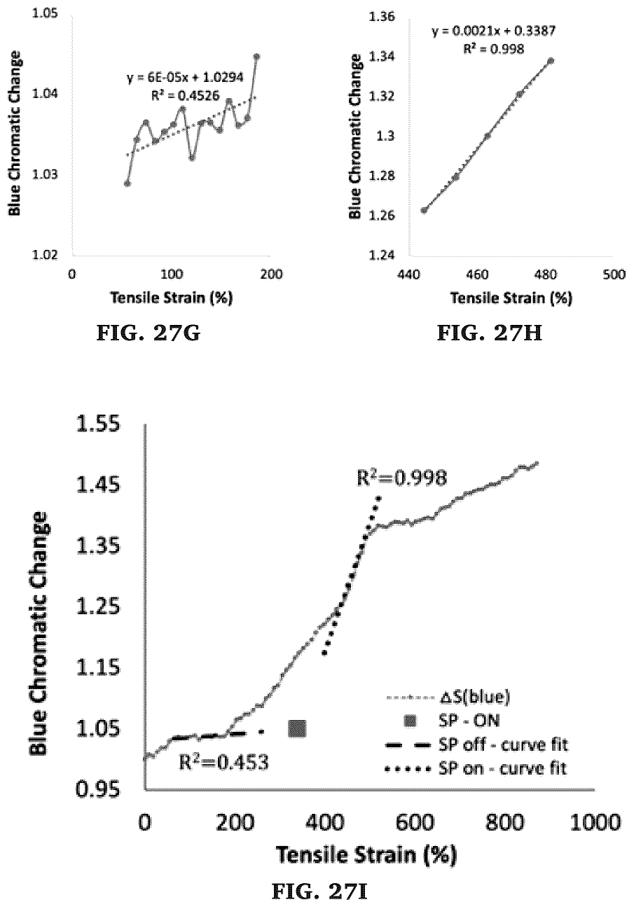

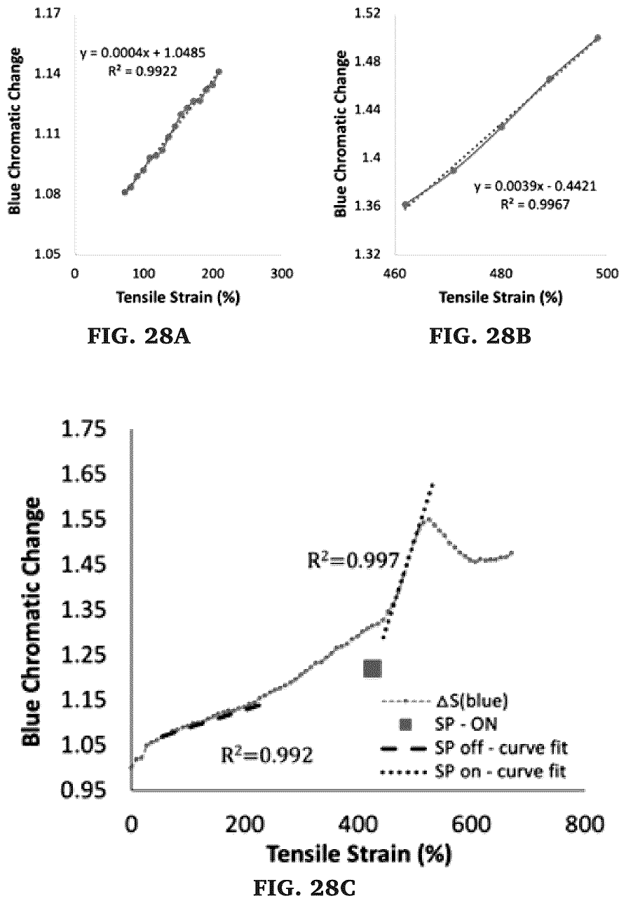

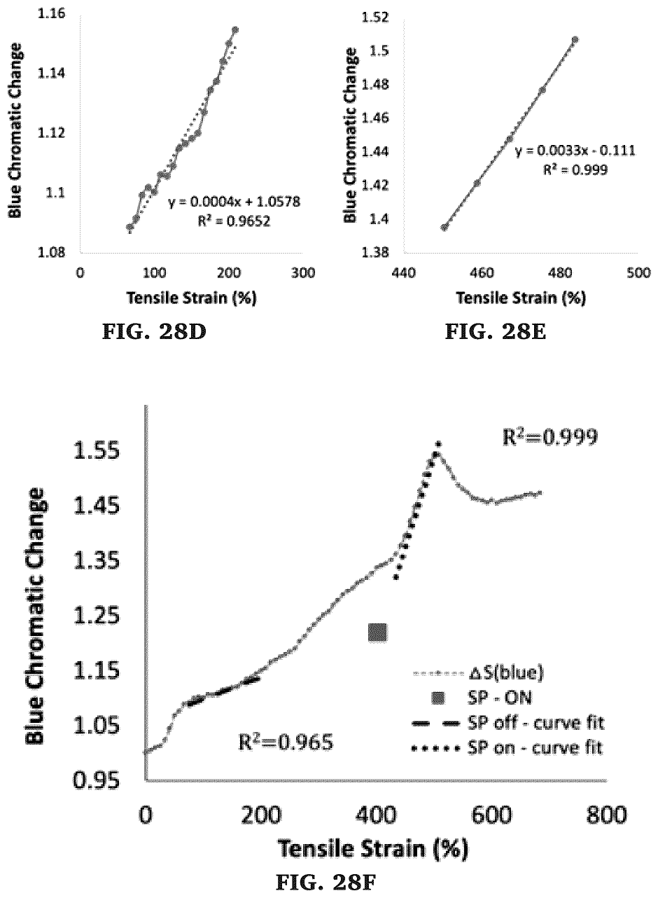

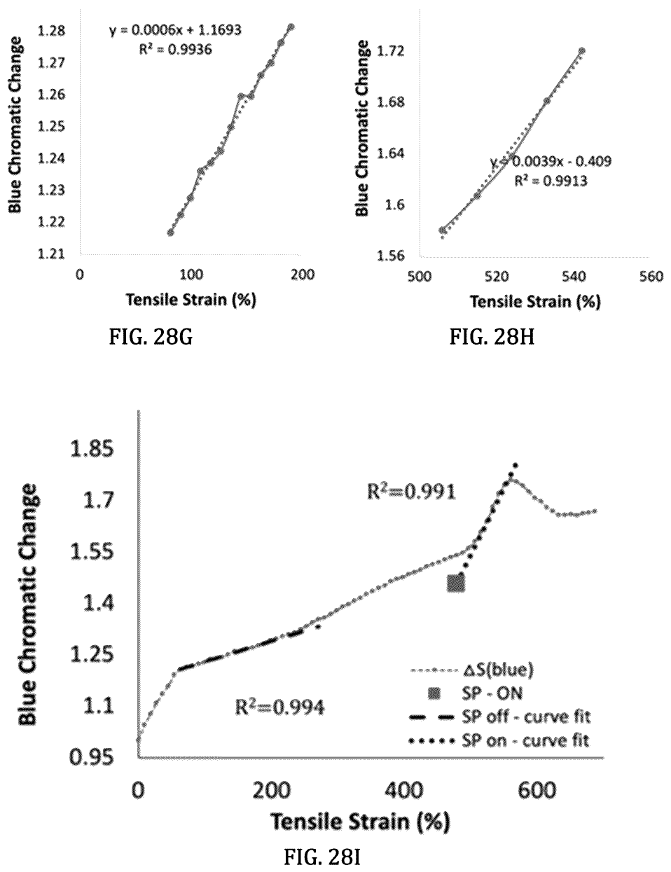

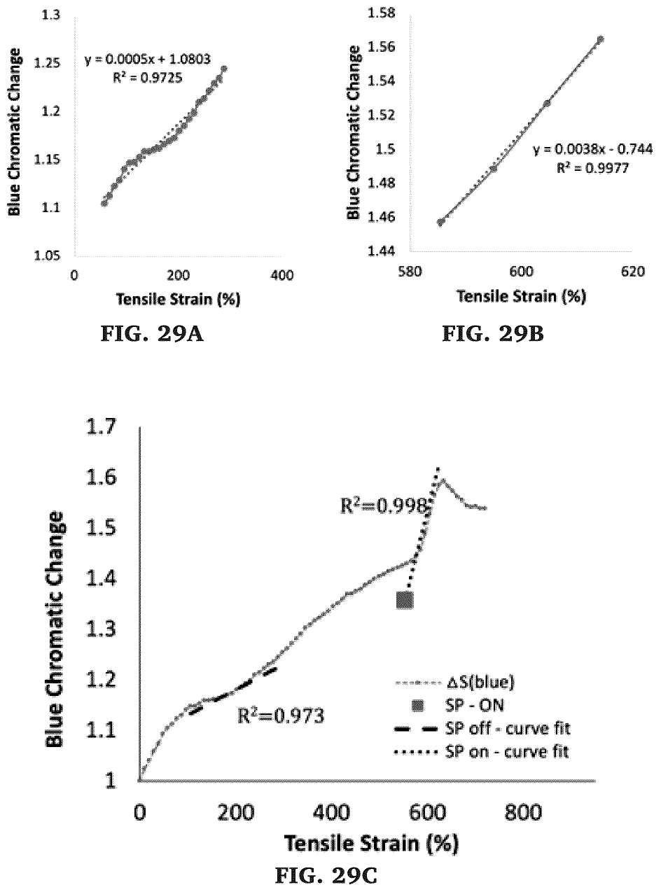

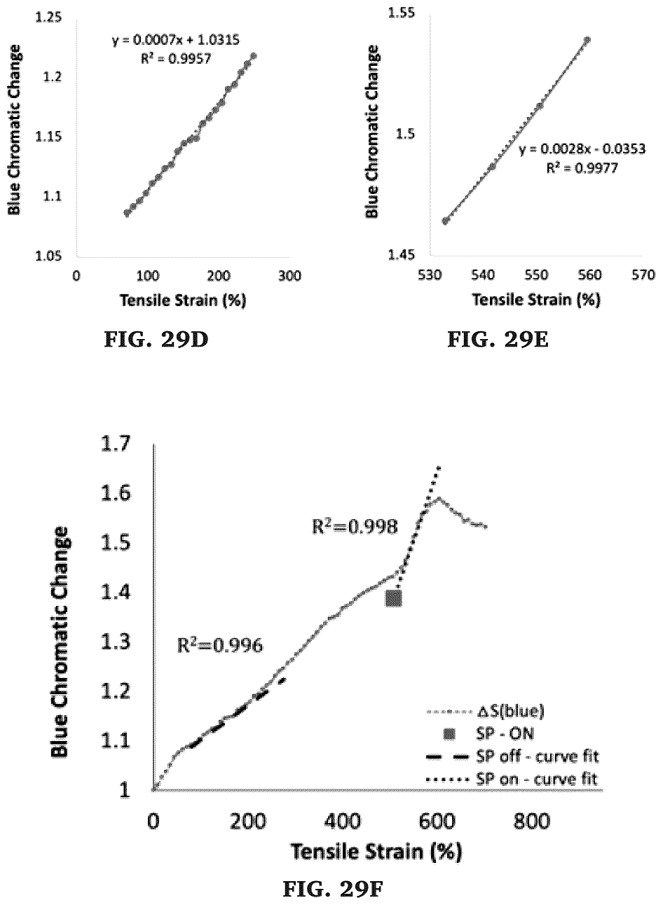

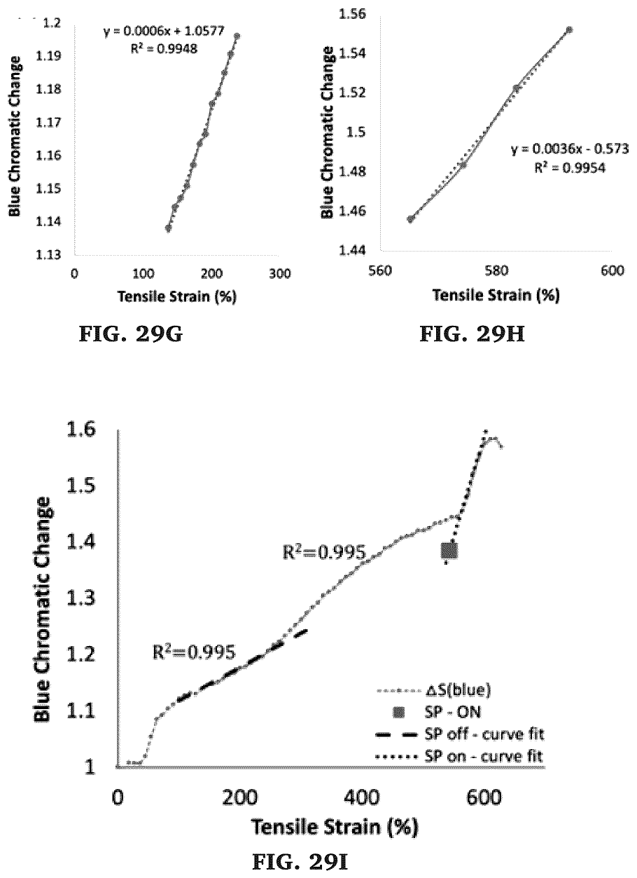

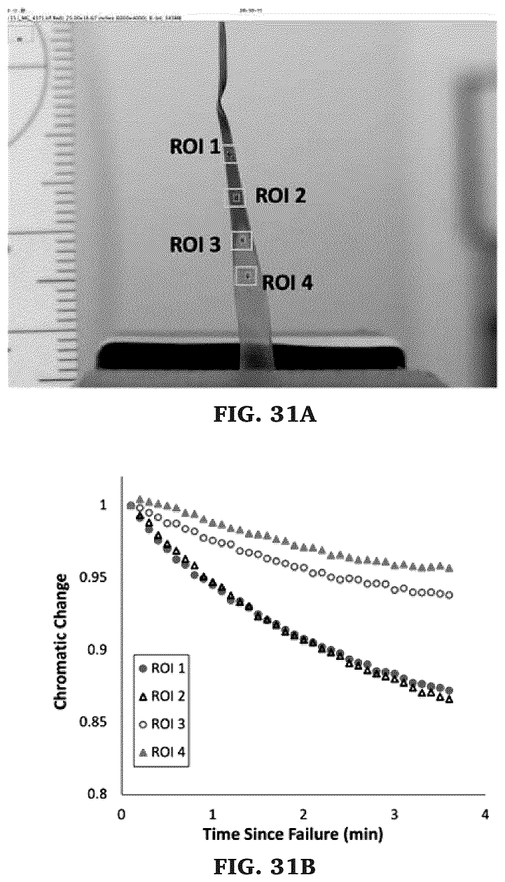

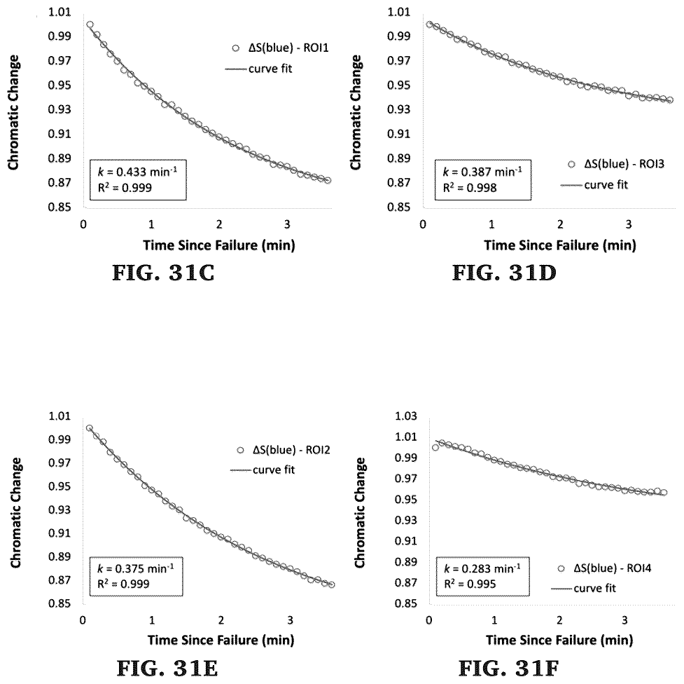

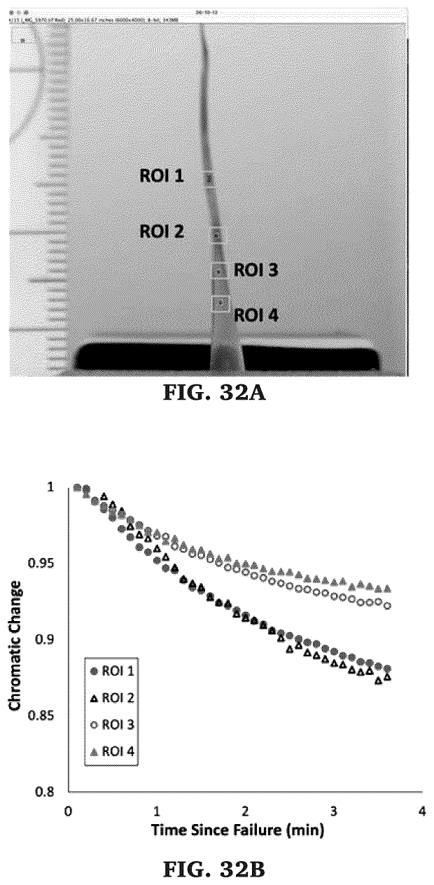

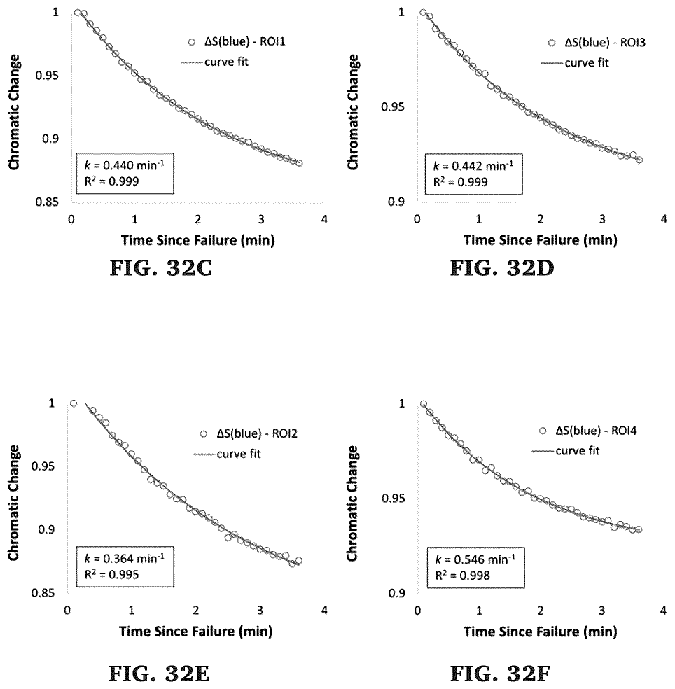

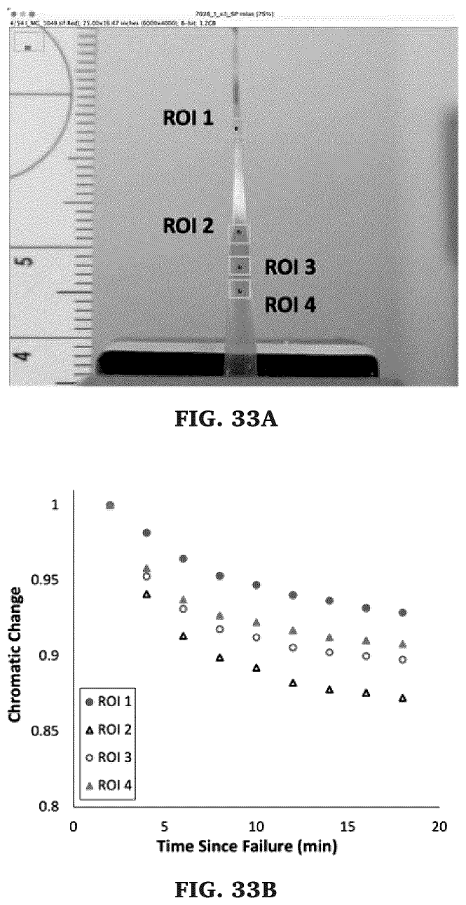

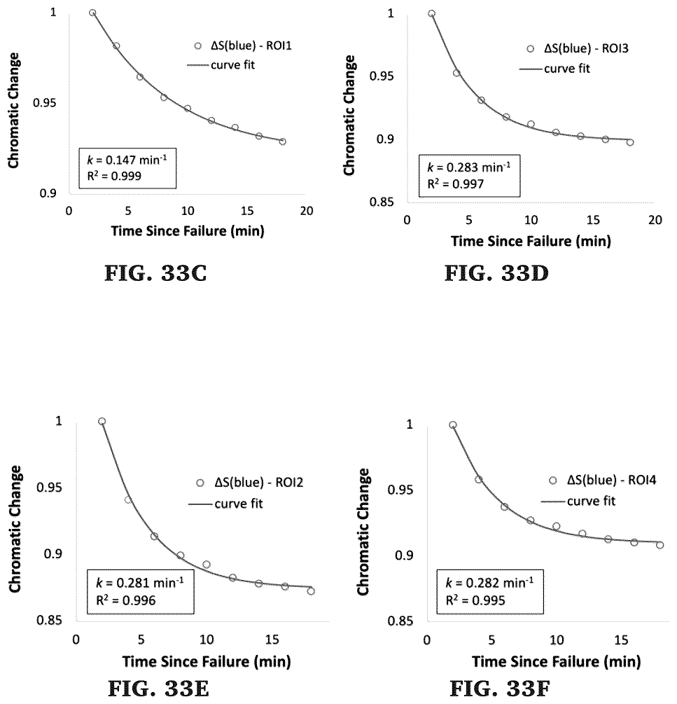

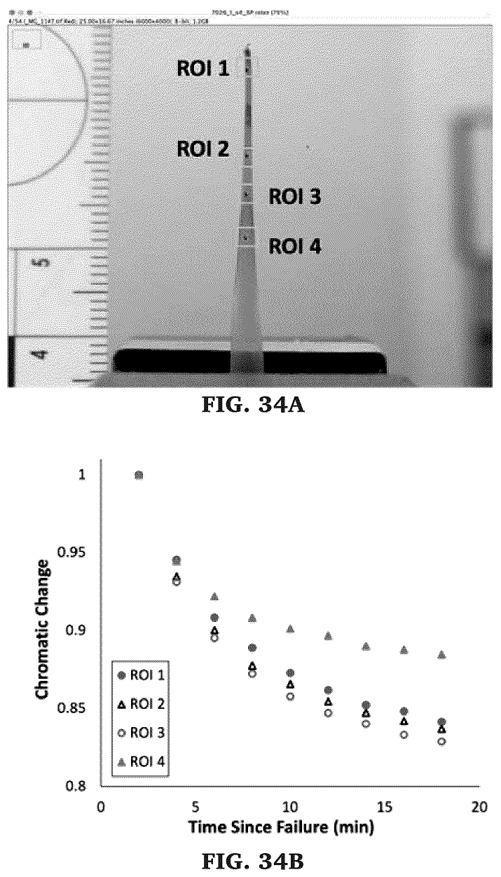

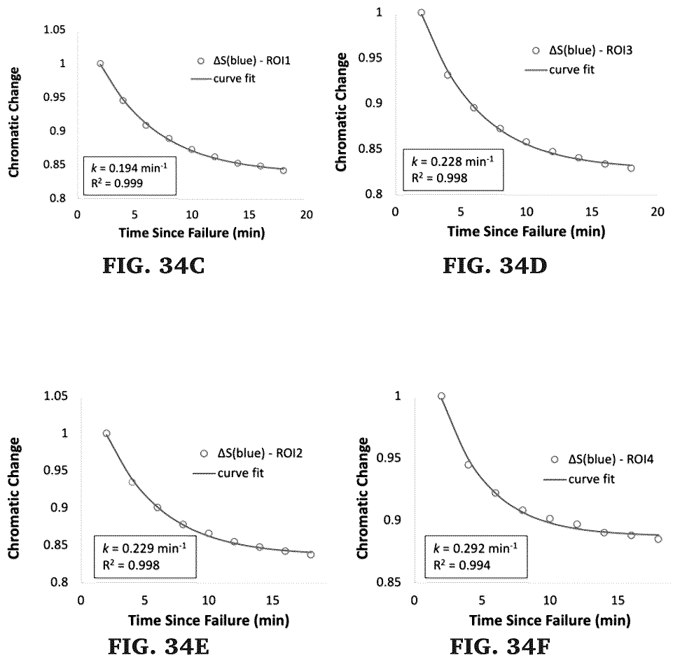

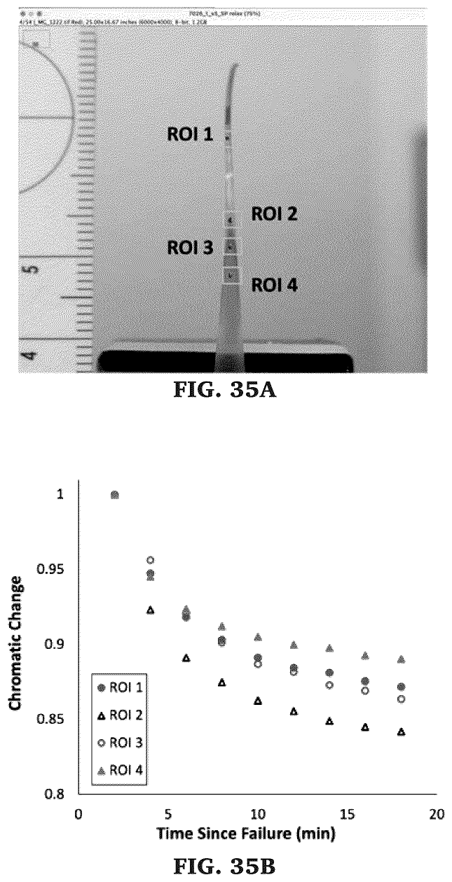

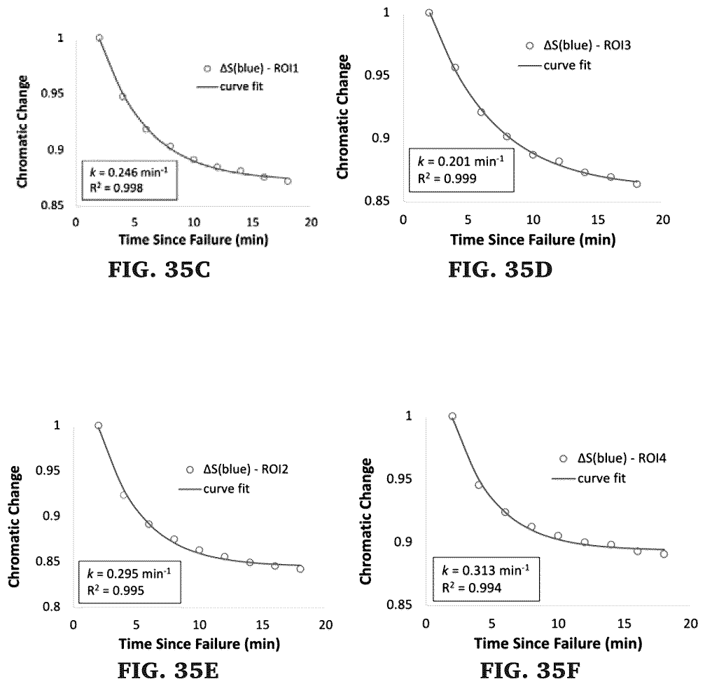

Before performing any regressions, however, all images within each experimental set were carefully inspected, and the image that displayed a significant increase in blue intensity was identified. The two activation regimes are observed readily in images of 1-SP (FIGS. 14A-C and FIG. 20A-B); all samples display an initial low level of SP activation (λ=300%), followed by intense color change (around λ=400-600%). All curve fittings are pictured in FIGS. 21-35. These FIGS show λ_{Act} increases for decreasing \dot{ε}, for both 1-SP and 1.5-SP (see, Table 3, Appendix), but λ_{Act} always lies within the first SH regime. The corresponding σ_{Act} typically occurs between 12 and 20 MPa (see, Table 3, Appendix), with a negligible \dot{ε}-dependence, as observed in other reports of SP-doped materials. (See, Y. Chen, C. J. Yeh, Y. Qi, R. Long, C. Creton, Sci. Adv. 2020, 6, eaaz5093; and Y. Lin, M. H. Barbee, C. C. Chang, S. L. Craig, J. Am. Chem. Soc. 2018, 140, 15969, the disclosures of which are incorporated herein by reference in their entirety). As noted above, the presence of necking and ultimate large deformations in these systems complicates a precise measure of true stress/true strain behavior. The true strains in the gauge region (where transitions in mechanochromism are monitored), however, can be obtained with good accuracy (albeit modest precision) by measuring the relative displacement in the images of marks placed on the gauge with a marker. Using this method, we find that the range of λ=300-500% (within which λ_{Act} is observed) corresponds to gauge strains of 400 to 500% (obtained with pixel analysis).

Most importantly, when the λ_{Act} extrapolations are plotted with σ_{True}(t)/\dot{ε} data in FIG. 5D, each of these points cluster on the same position of their respective curves, which confirms that this method of identifying λ_{Act} is valid for all elastomers at all examined \dot{ε}.

### 1.8. SP Recovery Kinetics

### 1.8.1. Color Persistence

The SP derivative employed in these systems is open only as a critical force is applied; typically, when force is removed, the activated merocyanine isomer relaxes immediately (in less than 0.1 s). The proton substituent para to the pyran oxygen offers little stabilization for the MC isomer, and this shifts the equilibrium towards SP. (See, e.g., M. H. Barbee, T. Kouznetsova, S. L. Barrett, G. R. Gossweiler, Y. Lin, S. K. Rastogi, W. J. Brittain, S. L. Craig, J. Am. Chem. Soc. 2018, 140, 12746, the disclosure of which is incorporated by reference in its entirety). In contrast, a more widely-used SP derivative has a nitro substituent that provides resonance stabilization, and the activated MC may persist for up to 1 h after removing force, depending on the polarity of the medium. (See, e.g., C. K. Lee, D. A. Davis, S. R. White, J. S. Moore, N. R. Sottos, P. V. Braun, J. Am. Chem. Soc. 2010, 132, 16107, the disclosure of which is incorporated by reference in its entirety).

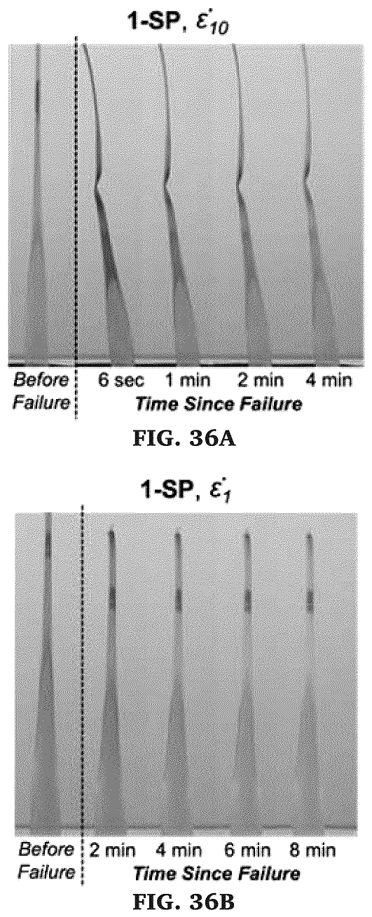

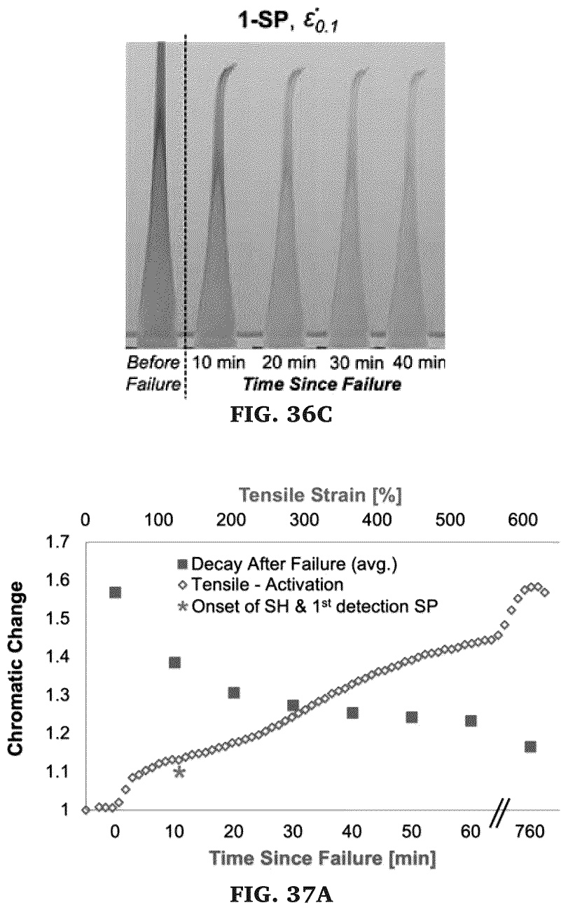

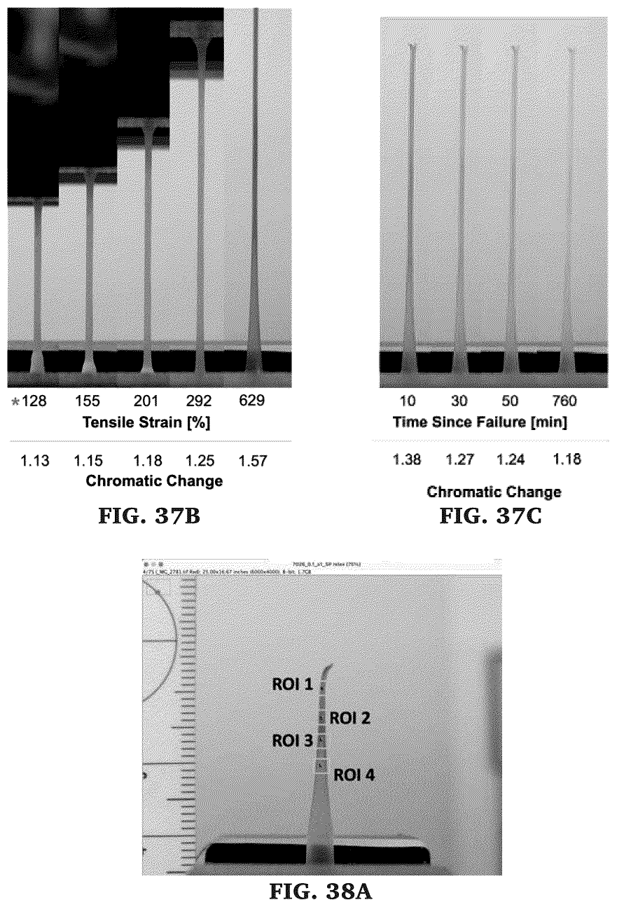

It is therefore notable that SP remains activated in these materials well after material failure releases the applied stress, apparently suspended in a force-activated, extended state. Moreover, the time needed for the mechanophore to relax back to its colorless SP isomer depends on \dot{ε} (FIG. 36A-C). Indeed, after stretching at \dot{ε}_{0.1}, the majority of the blue color fades over the course of several hours, and a small fraction (≈1-3%) is still visible even 12 h later (FIG. 37A-C). Similar behavior is observed after stretching at \dot{ε}₁₀, but the fading occurs over the timescale of minutes. The slow relaxation of SP in the thiol-yne elastomer after material failure indicates there are underlying changes in polymer morphology that are governing the SP elastomer stress relaxation kinetics.

### 1.8.2. Dependence of SP Recovery Time on Strain Rate of Activation

Images, which were captured at regular intervals with the intervalometer, also provided the raw data to study SP elastomer relaxation kinetics. ΔS_{blue} intensity was calculated in images after material failure to determine the decay kinetics of the blue signal. For these tests, 4 different ROI of identical size and shape were selected for each sample to account for any error associated with camera focus and differences in localized stress due to polymer necking. Then each set was normalized such that ΔS_{blue}=1 for the first image acquired after material failure. Data for each ROI were fit to an exponential decay curve in Matlab, according to Equation (3):

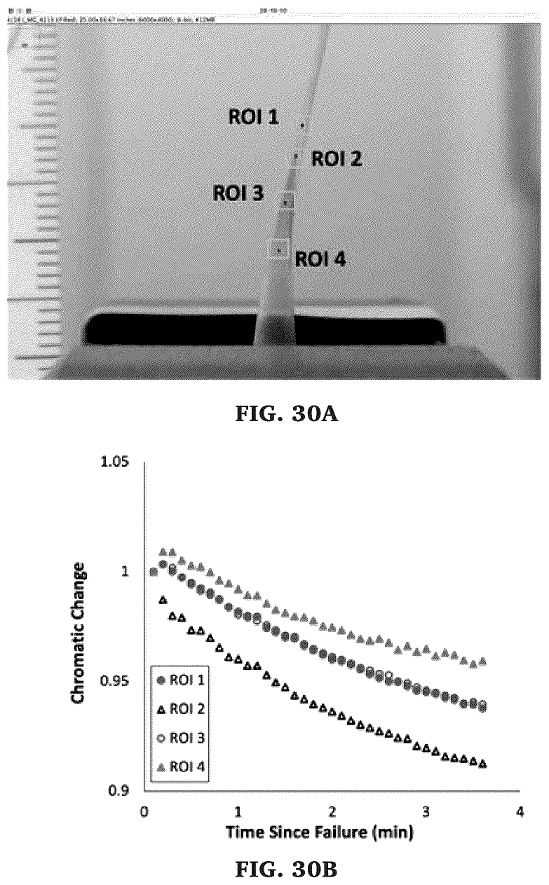

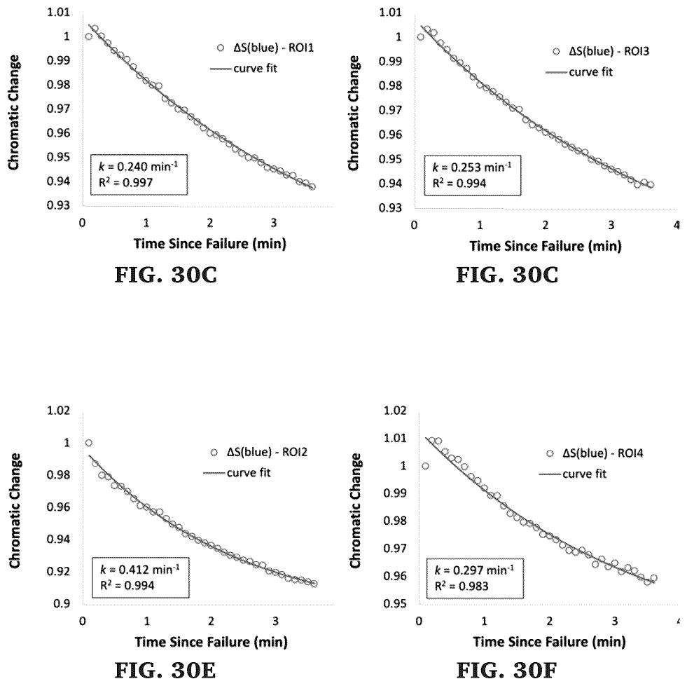

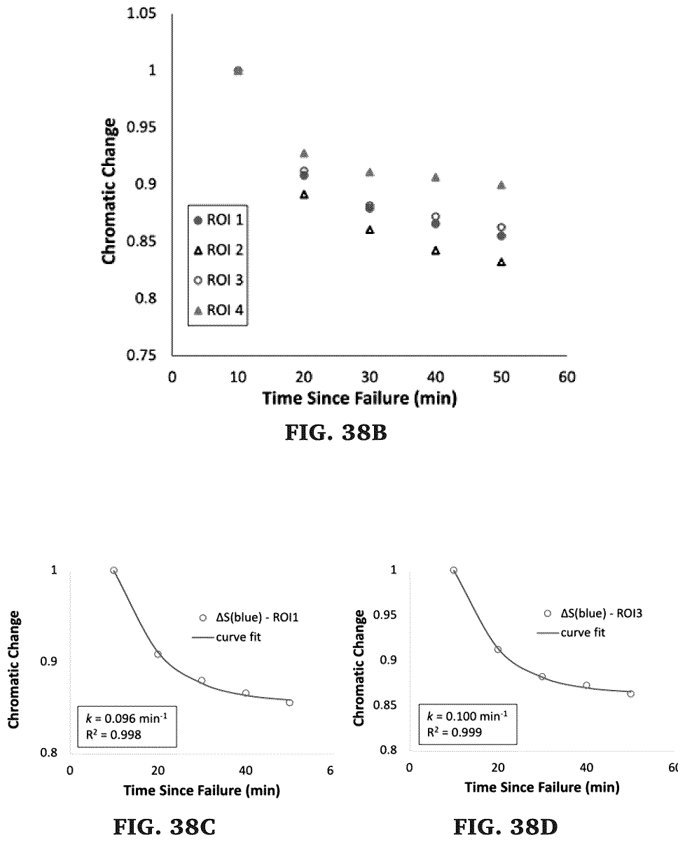

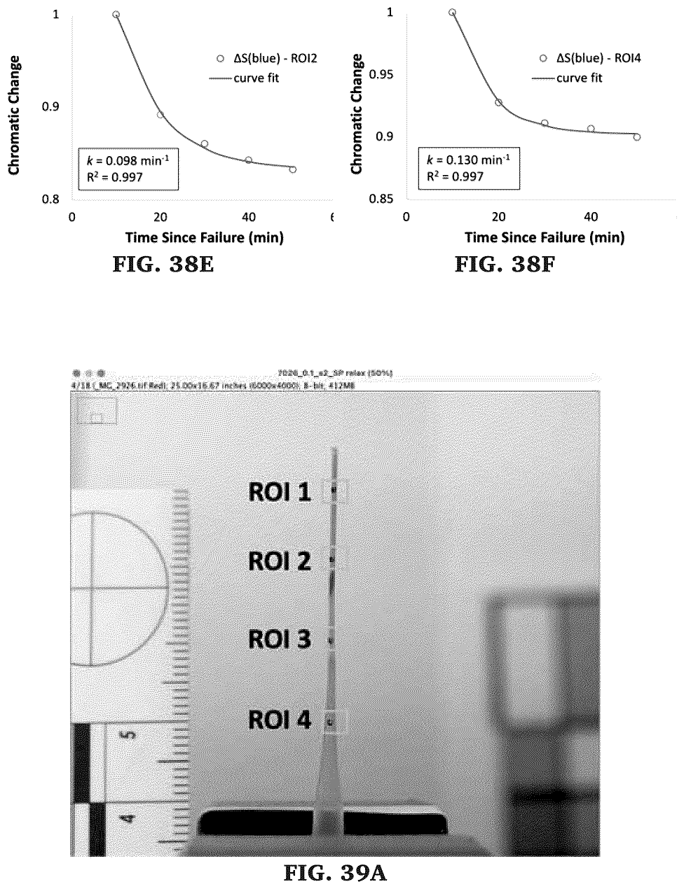

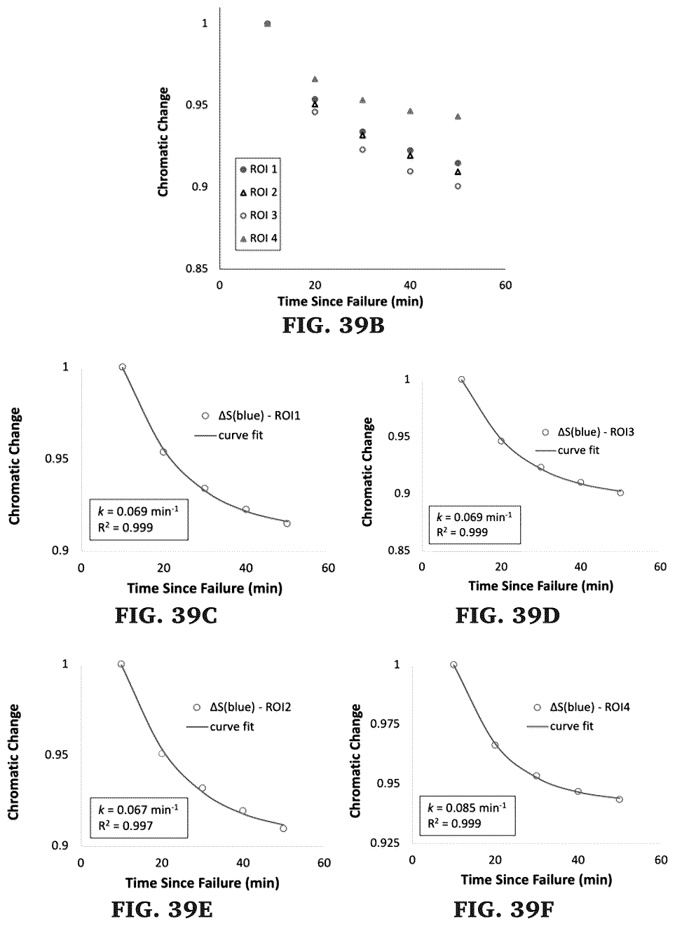

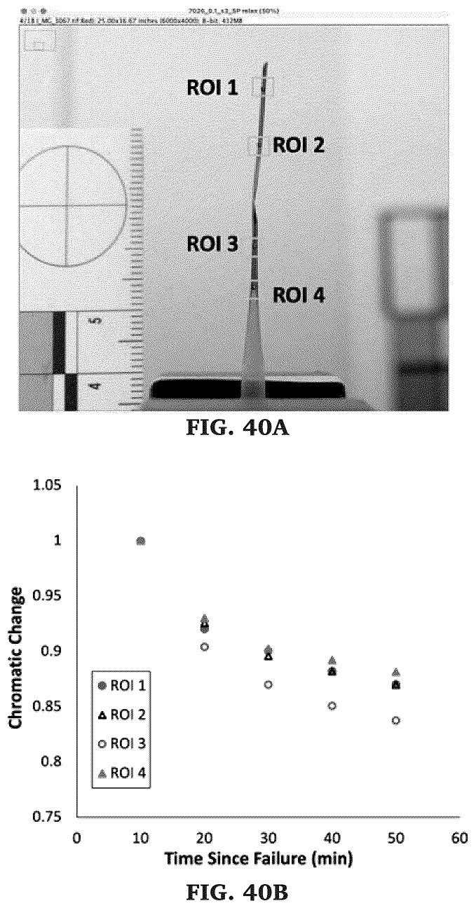

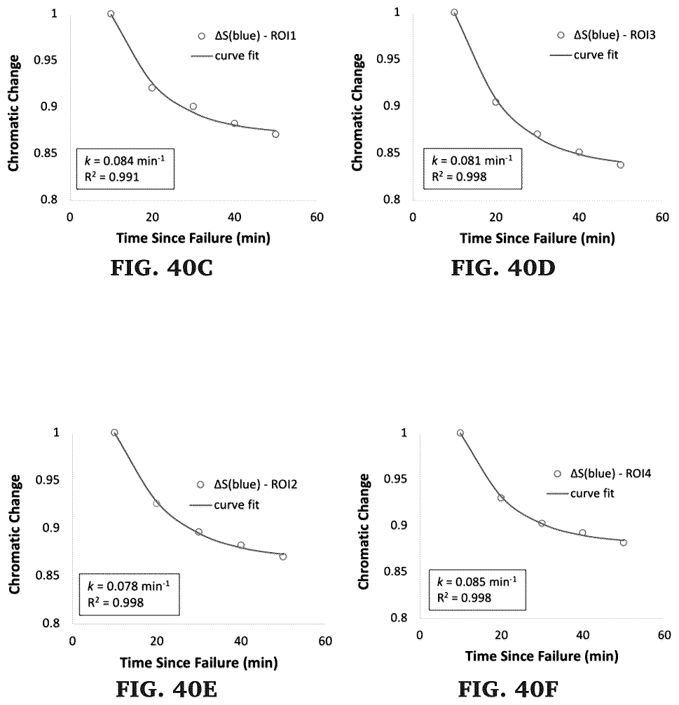

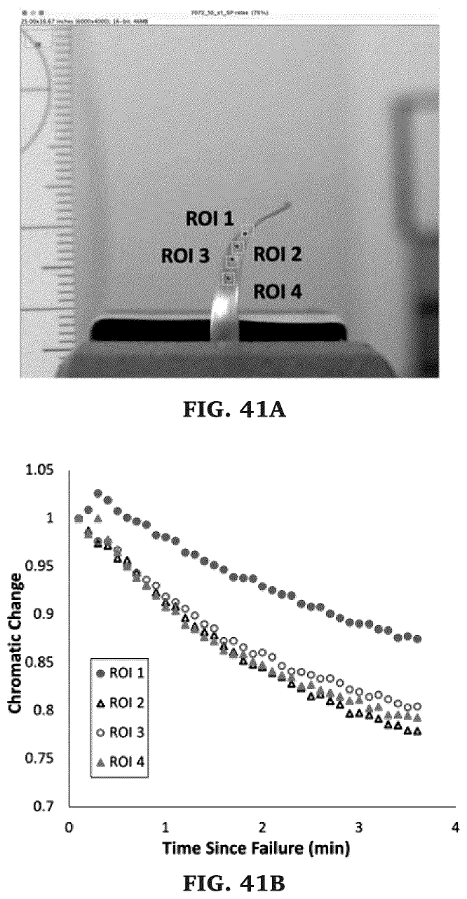

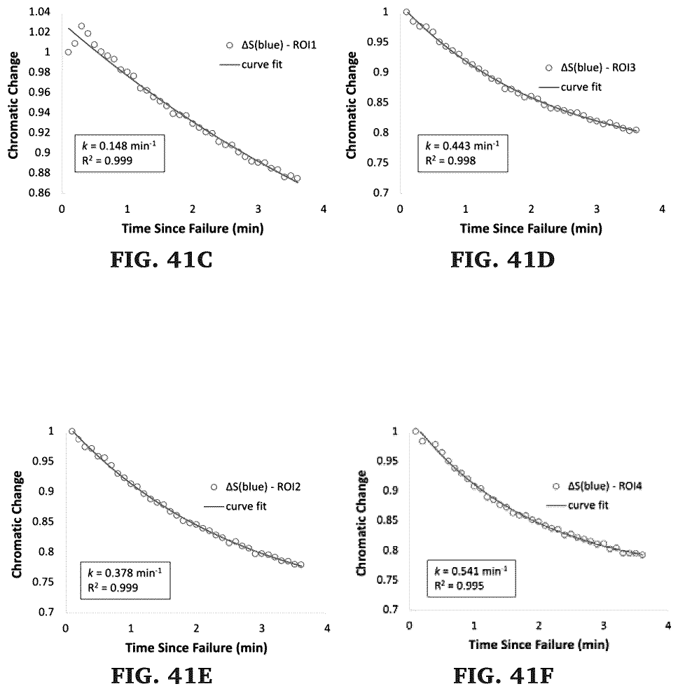

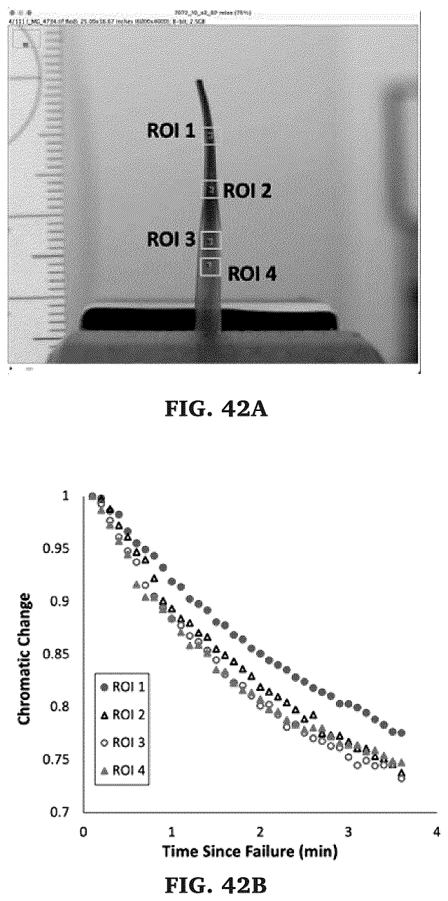

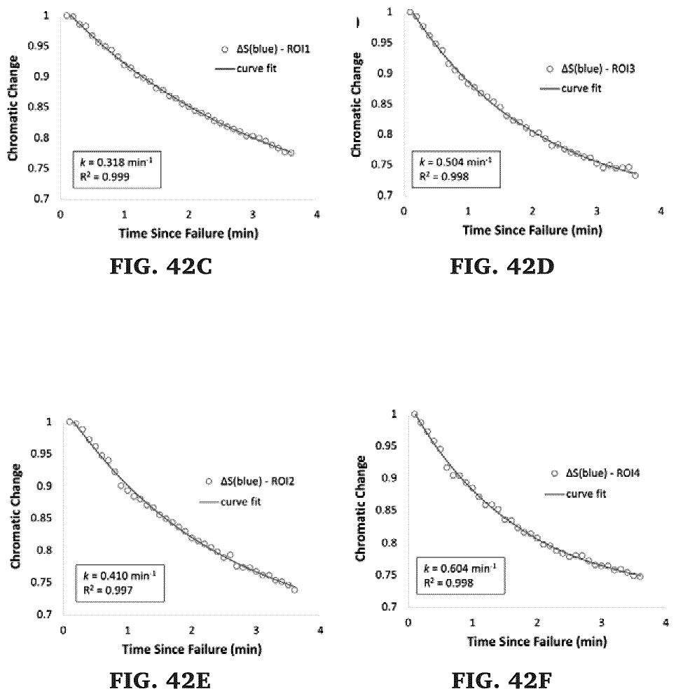

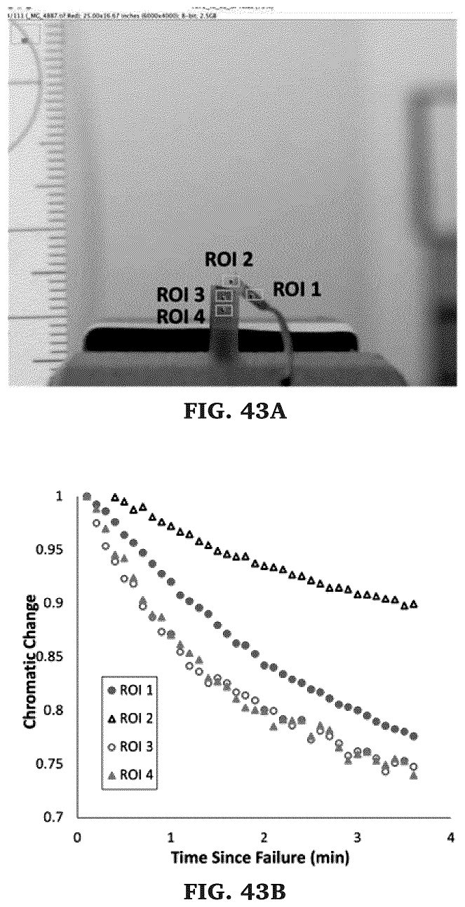

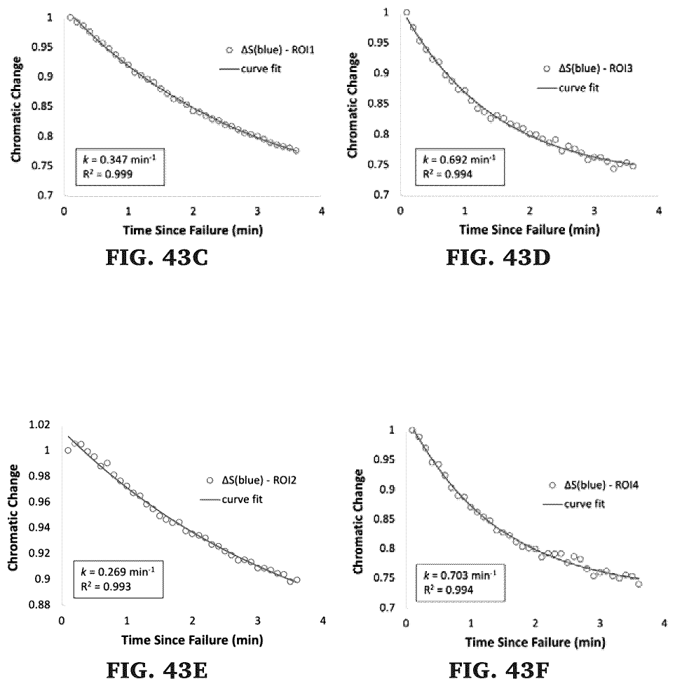

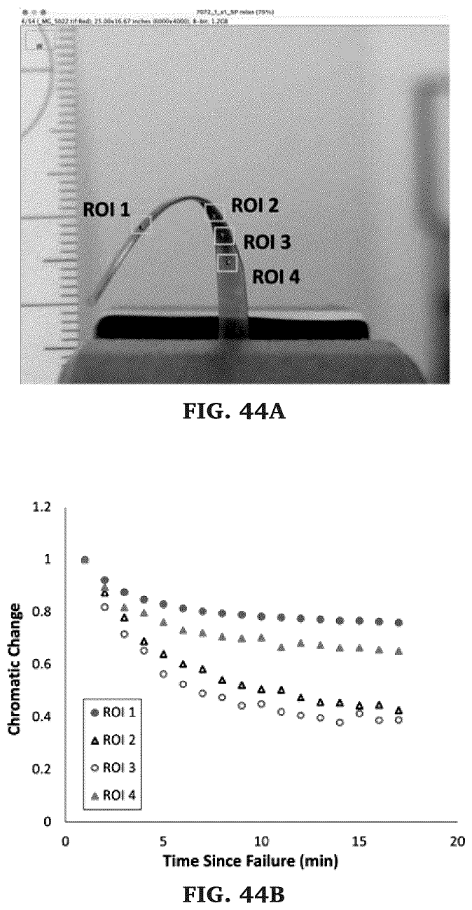

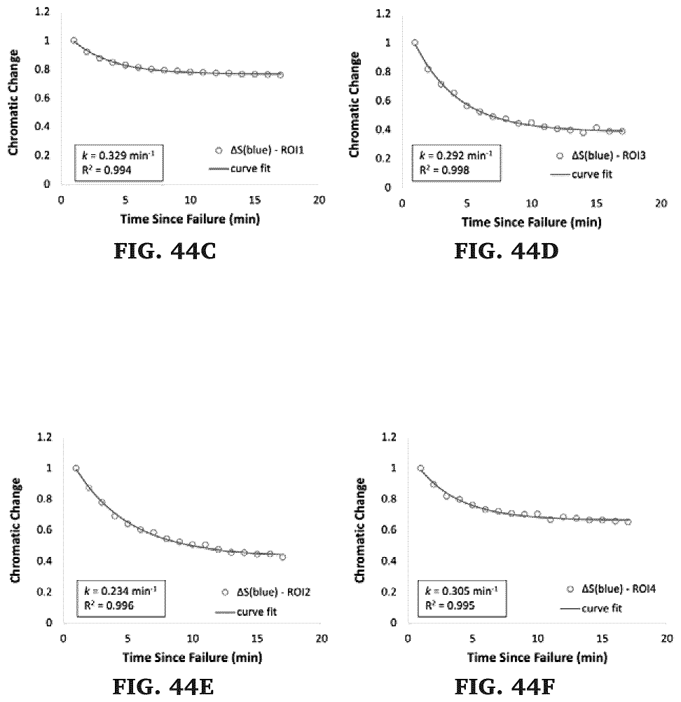

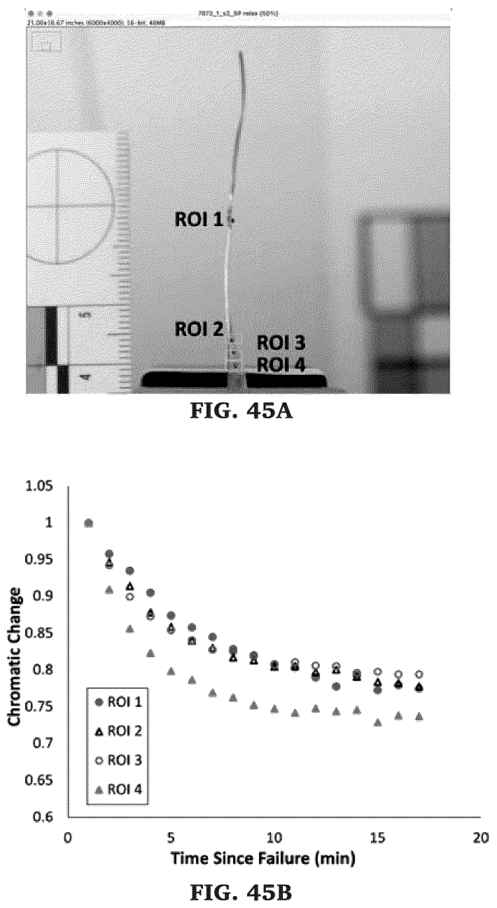

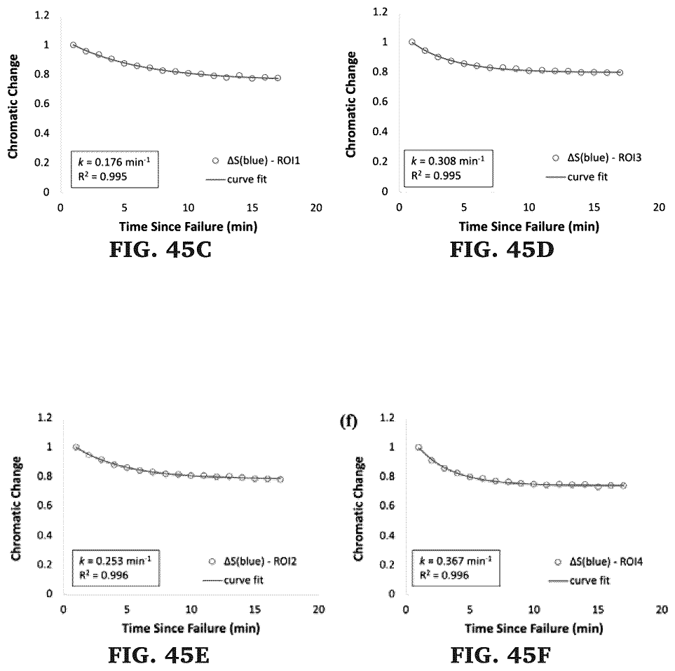

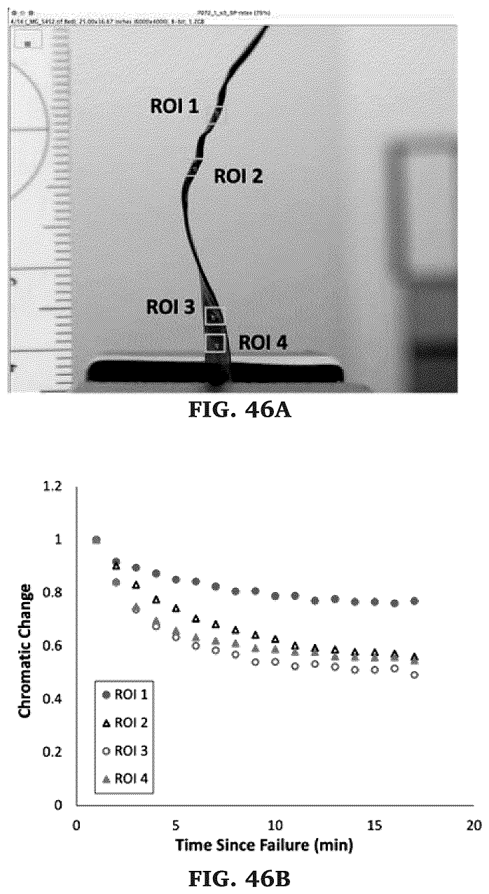

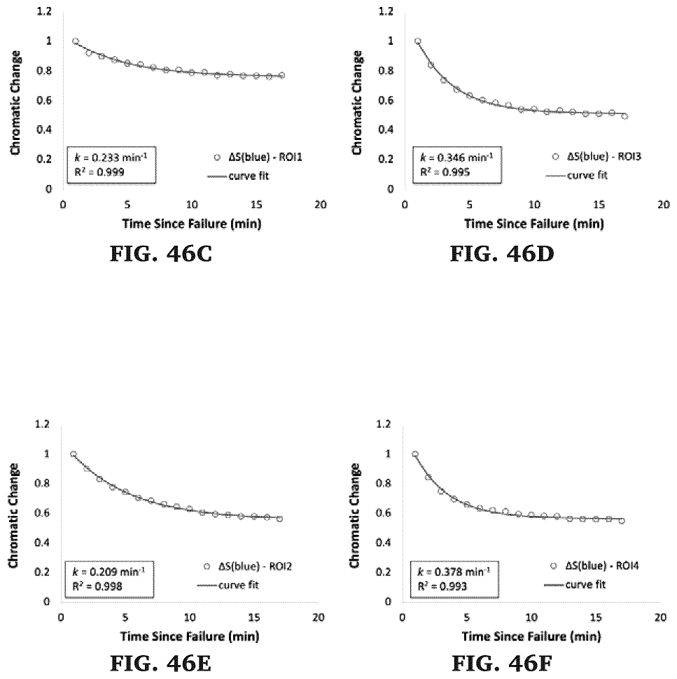

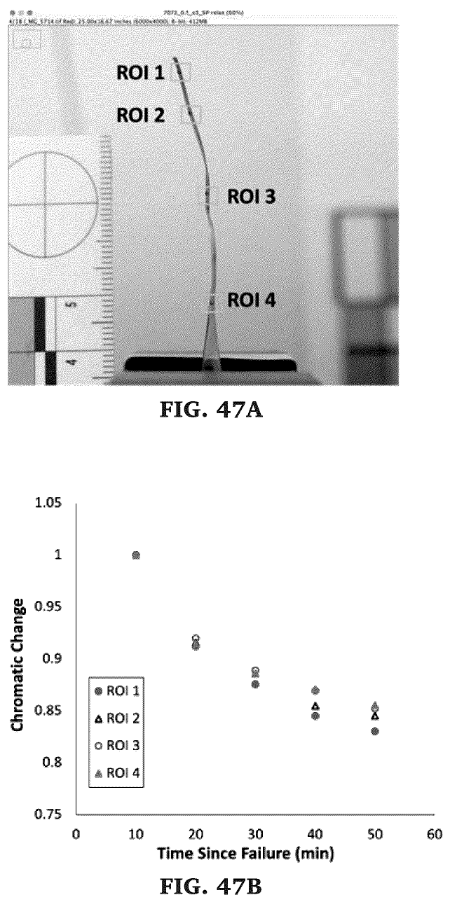

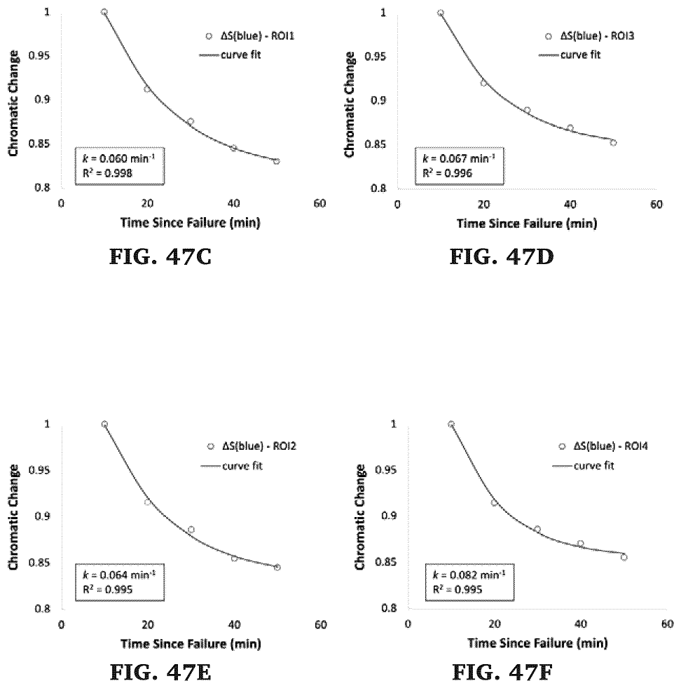

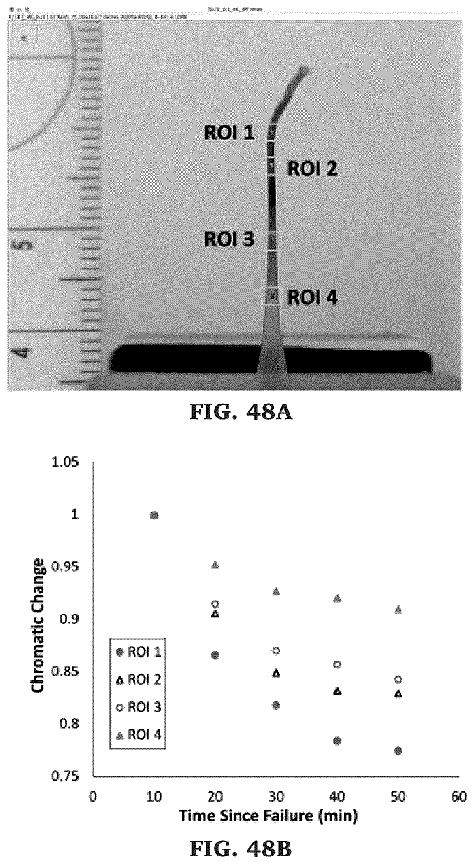

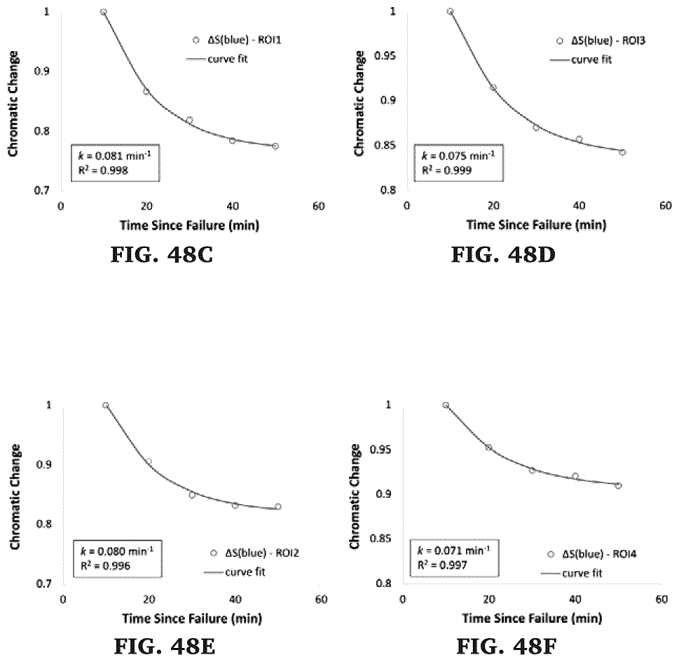

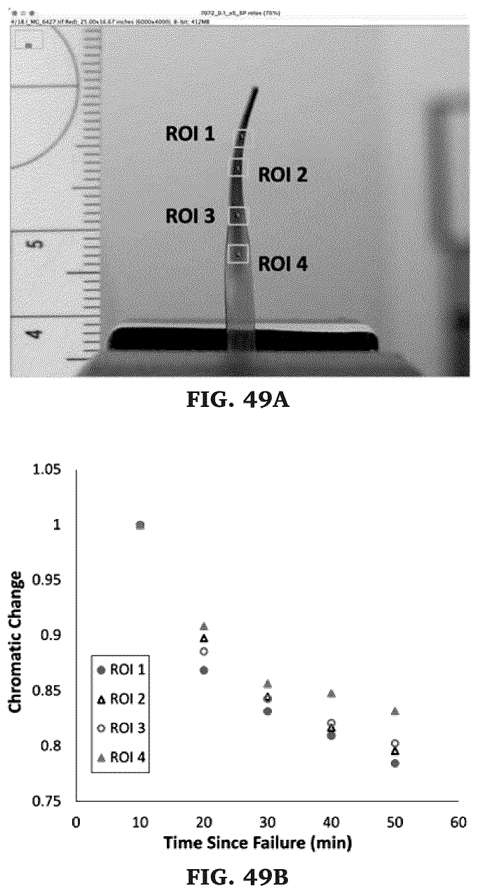

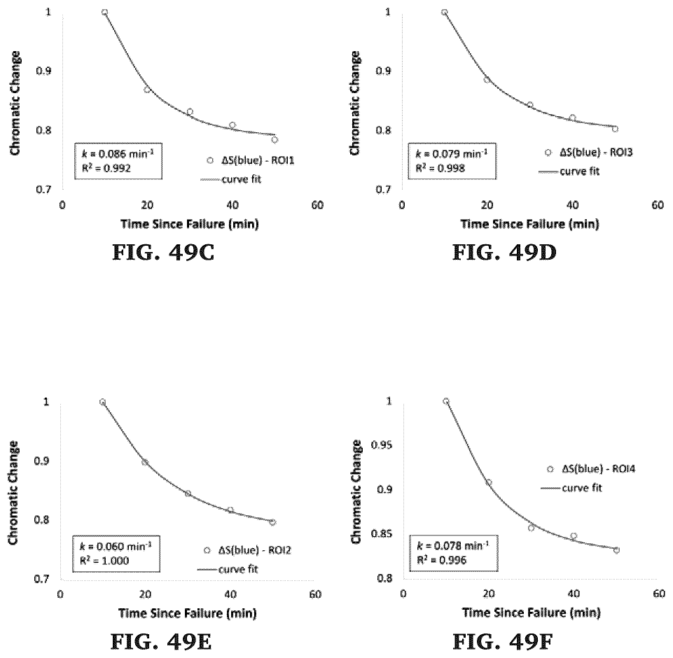

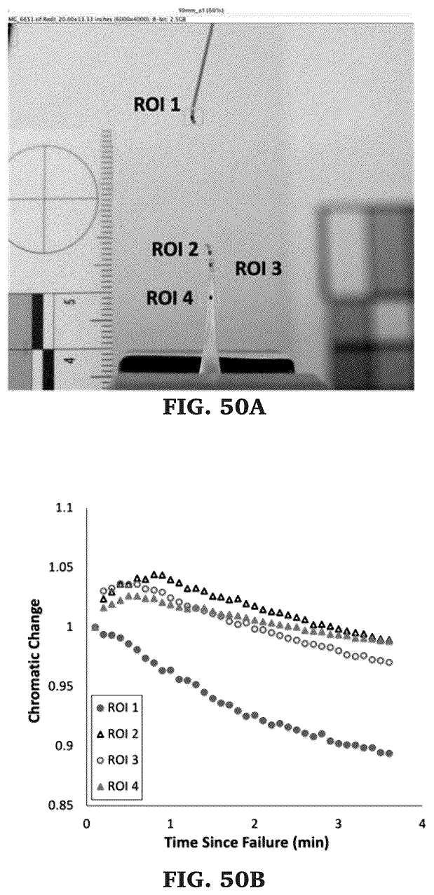

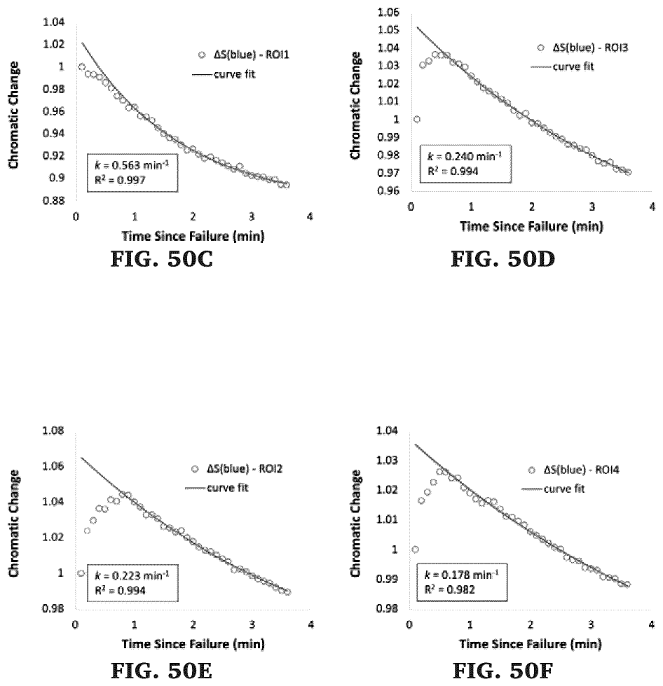

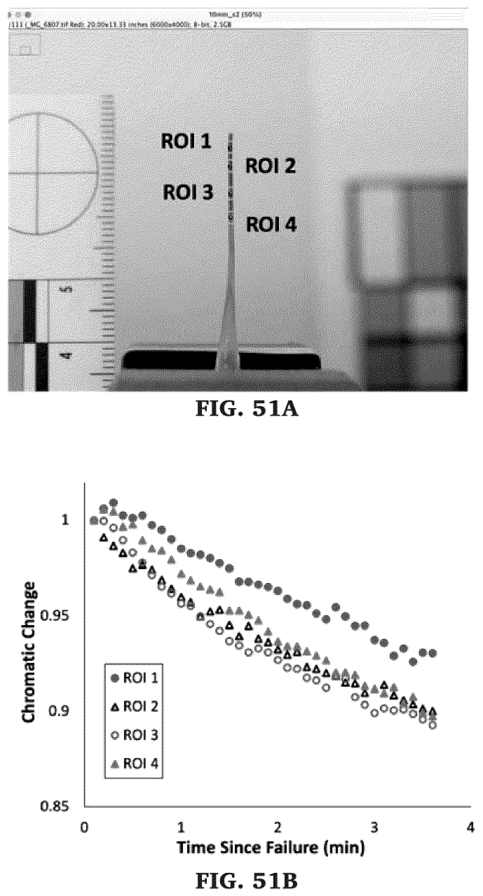

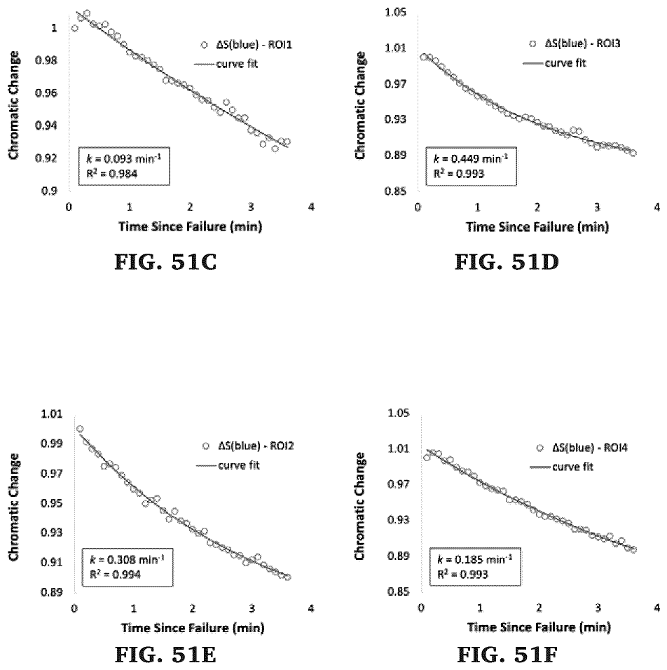

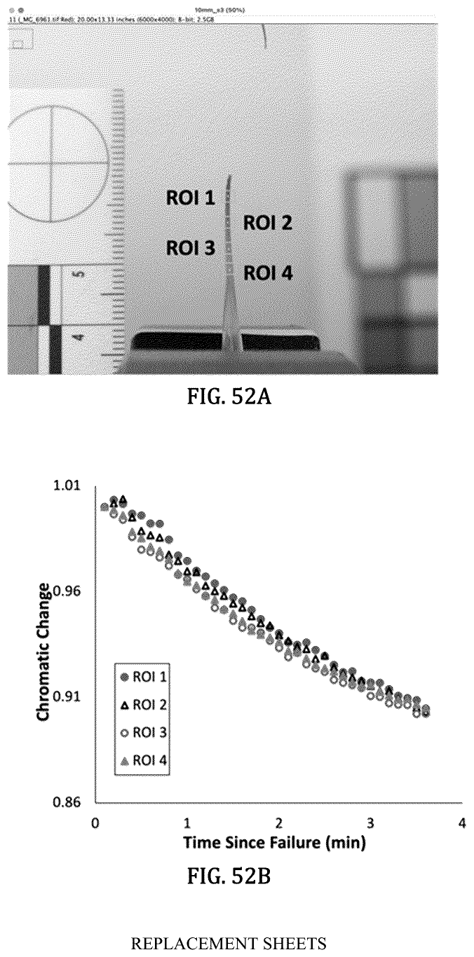

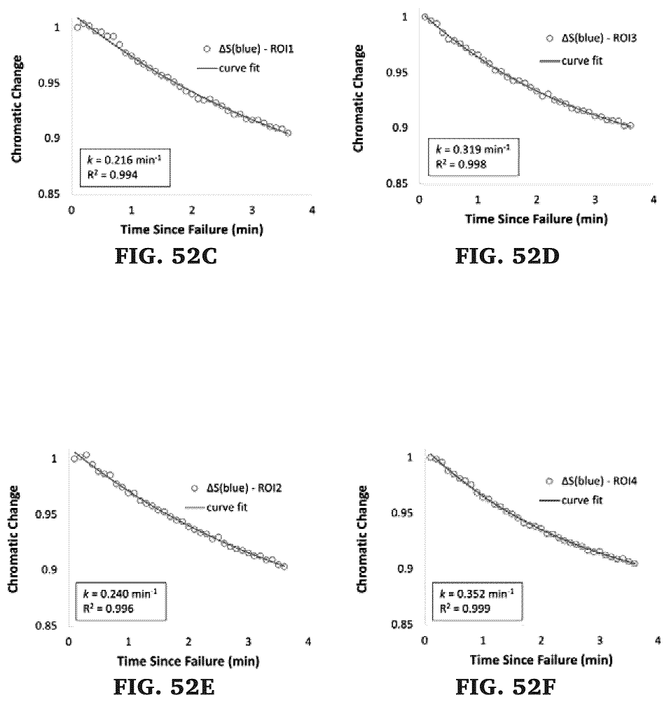

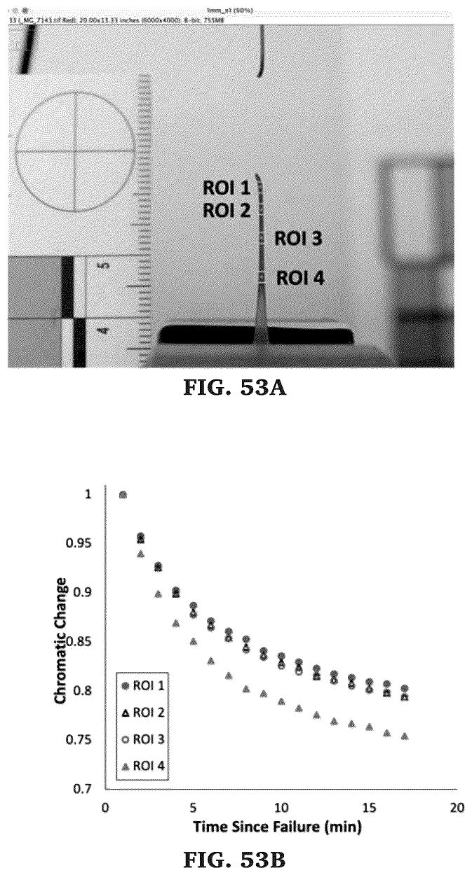

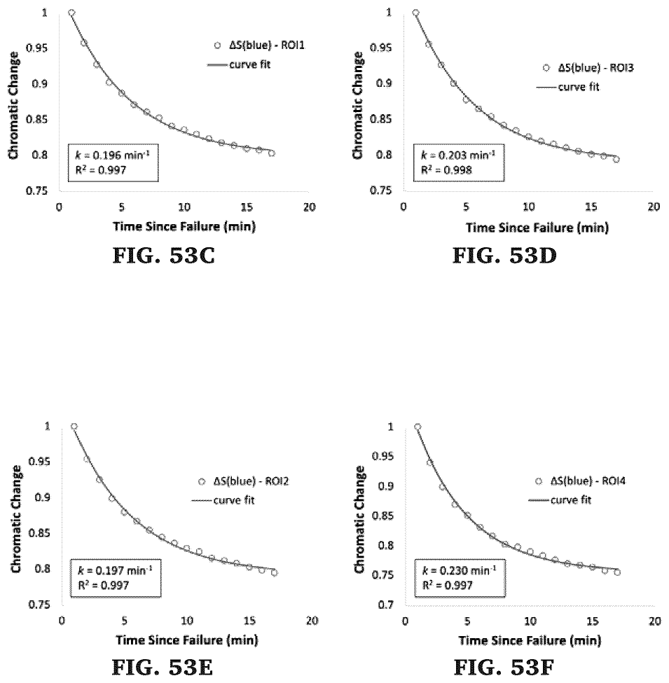

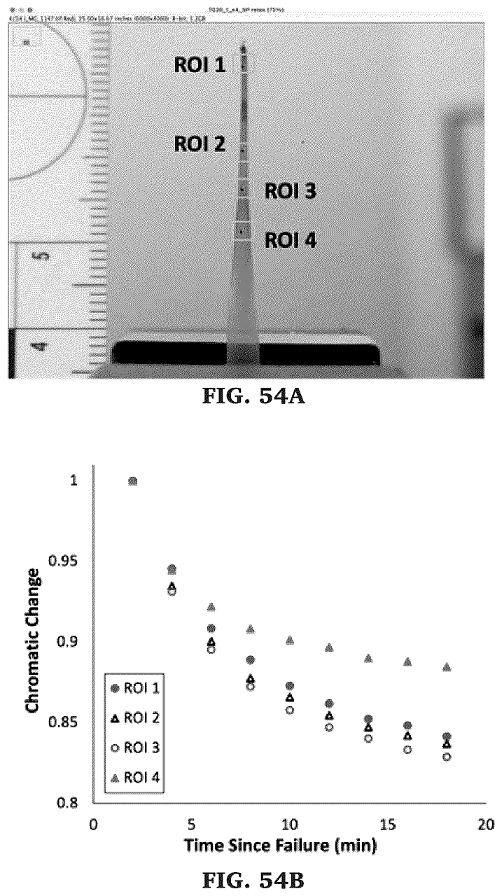

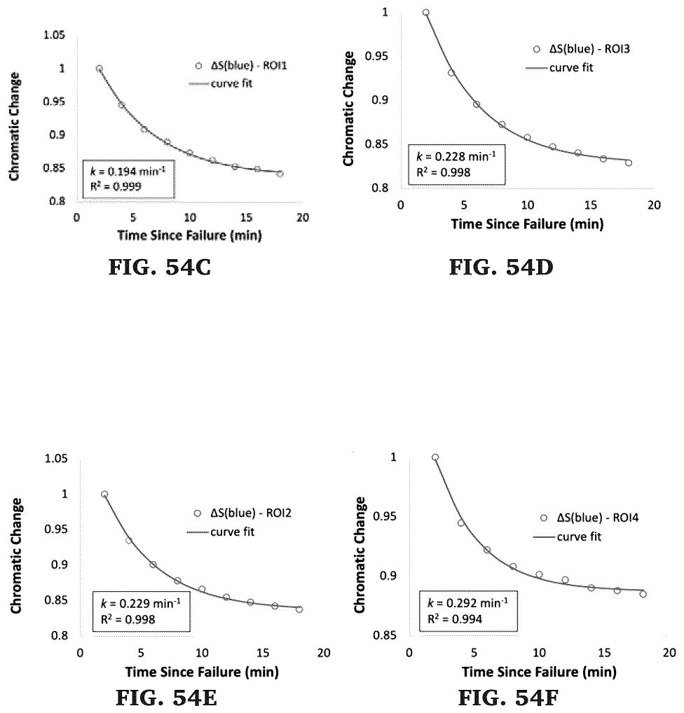

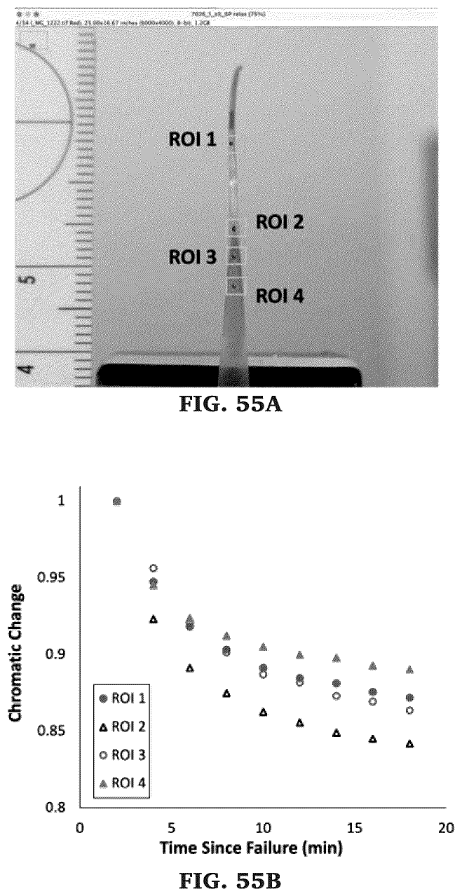

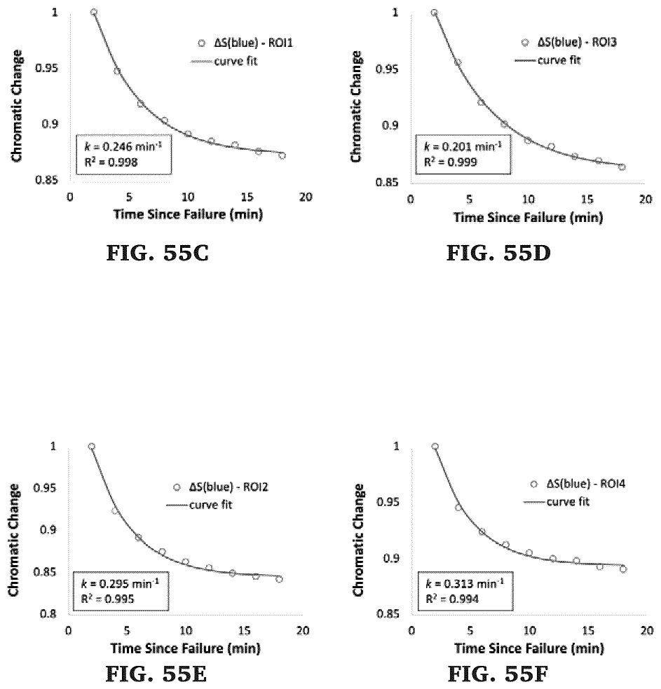

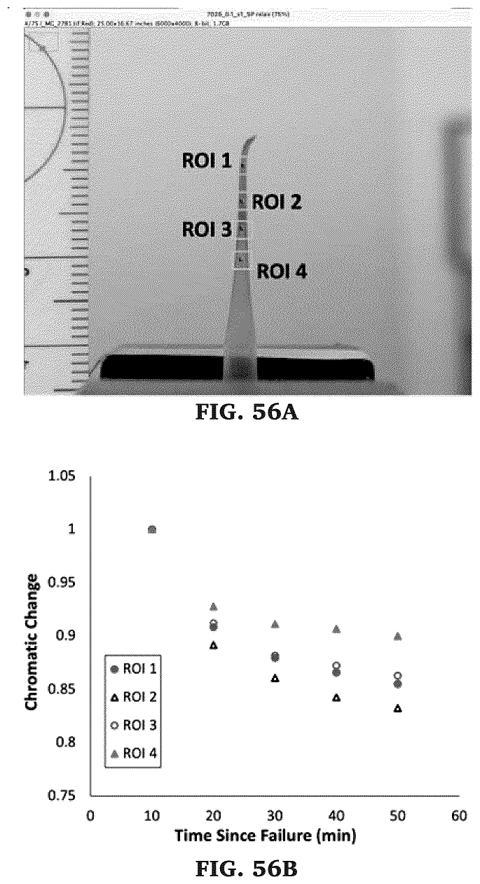

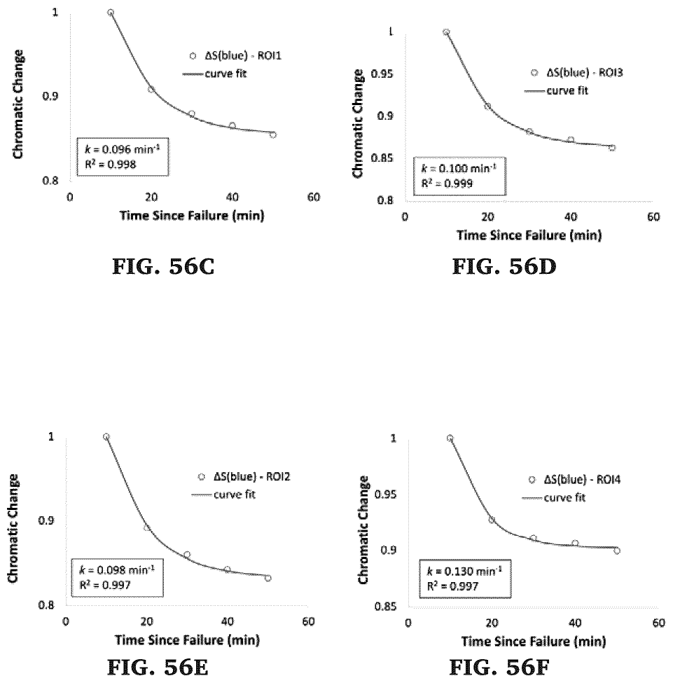

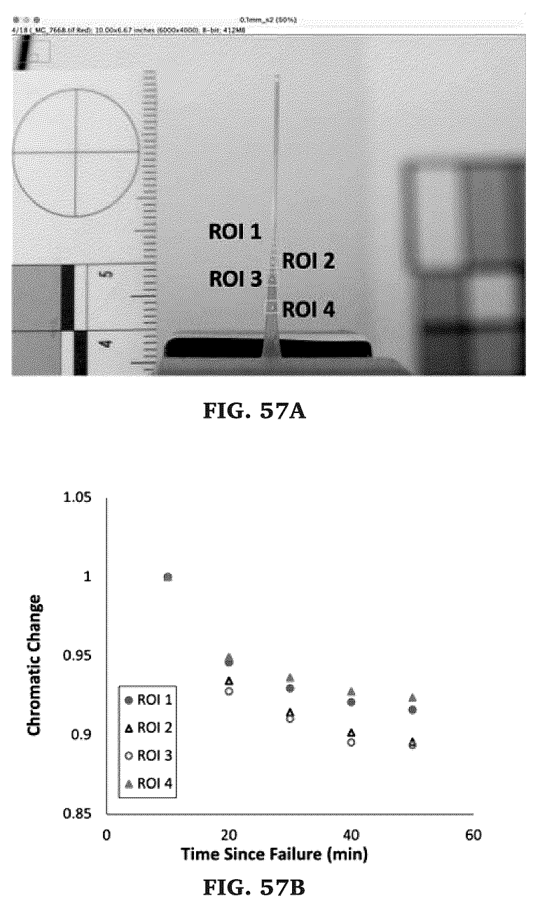

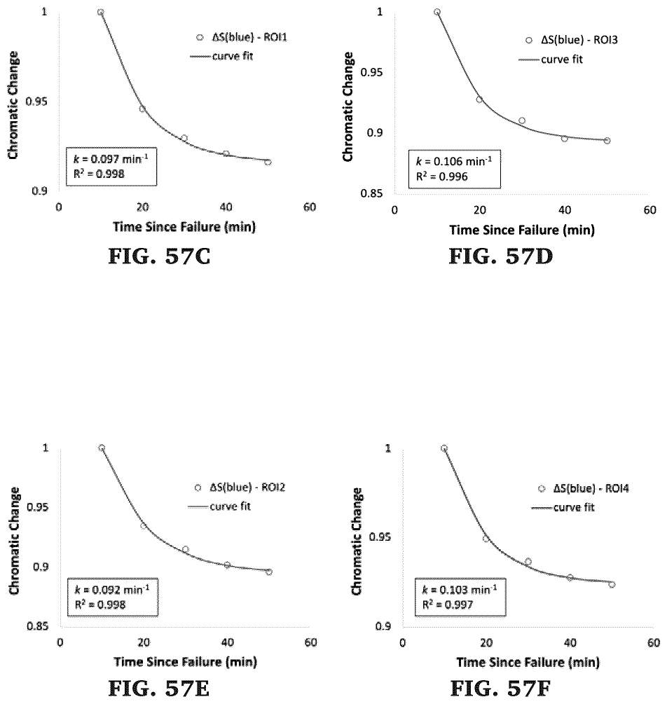

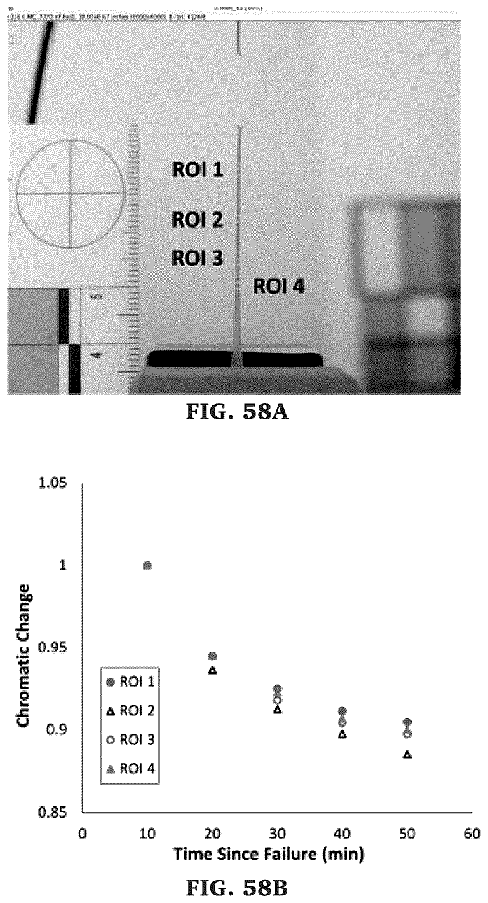

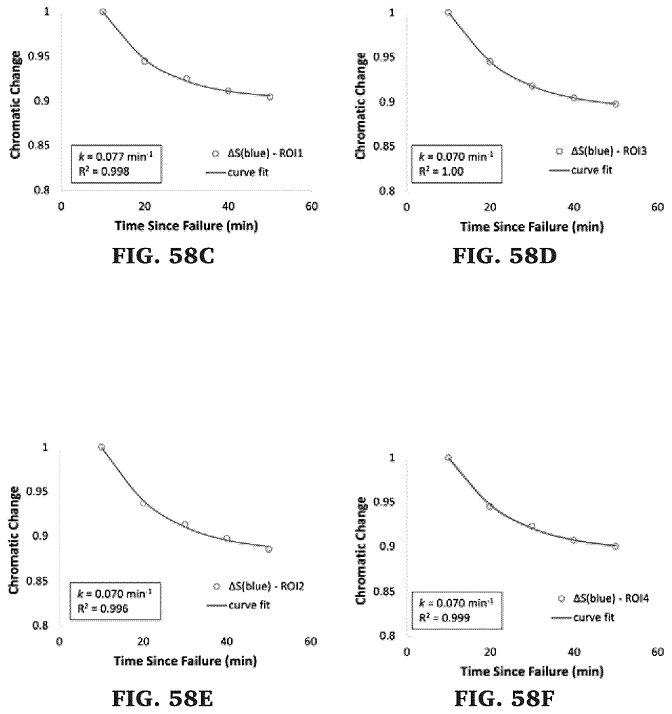

ΔS_{blue}=a*e^{(-kt)}+c (3)

where a and c are constants, k is the kinetic decay constant, and t is the elapsed time since failure. It is important to reiterate that normalizing ΔS_{blue} facilitates direct comparison between data sets and does not artificially skew sample data. This was confirmed by calculating k before and after normalization, which were found to be identical. All kinetics results are reported from n=12 samples (4 ROI per sample, and 3 samples per strain rate), and all data fittings are included in FIGS. 30-35, 38-58.

Relaxation kinetics data display both fast and slow phases, and all sample data curves display tails that deviate from the 1-phase curve fittings. This tail deviation begins to occur at 3.5, 17, and 50 min after failure for samples tested at \dot{ε}₁₀, \dot{ε}₁, and \dot{ε}_{0.1}, respectively. For the sake of simplicity, the results presented here are only for single-phase exponential fits and do not include the tail data. Additionally, immediately after failure, some samples display an initial fast increase in ΔS_{blue} as the material becomes a darker shade of blue; this unloading effect, which can be seen in FIG. 36A, has been well-documented in other SP-based materials, and it is likely representative of a change in volumetric concentration after material failure. There is \dot{ε}-dependence for this effect, as it is apparent only for samples tested at \dot{ε}₁₀. Kinetics fittings for any samples showing this effect used data only after ΔS_{blue} begins to decrease; this occurs usually within 12 s after failure, and examples of this effect are depicted in FIG. 30A.

Average decay constants (n=12) are listed in Table 3 (Appendix). For 1-SP tests at \dot{ε}₁₀, there was an average decay constant of 0.373±0.074 min⁻¹, which corresponds to a 1.9 min half-life. When 1-SP was tested at \dot{ε}₁ and \dot{ε}_{0.1}, decay constants decrease to 0.249±0.014 min⁻¹ and 0.087±0.017 min⁻¹, respectively. The corresponding half-lives for 1-SP are 1.9 min, 2.8 min, and 8.0 min for the three \dot{ε}. 1.5-SP showed similar behavior, with average decay constants of 0.488±0.025 min⁻¹, 0.285±0.008 min⁻¹, and 0.074±0.005 min⁻¹; the corresponding half-lives were 1.4, 2.4, and 9.4 min. Notably, there is more variation observed in k for samples strained at \dot{ε}₁₀ than for those strained at \dot{ε}₁ and \dot{ε}_{0.1}. This is likely because images captured after failure are more likely to be out of focus at higher strain rate tests. At this point, the broken elastomers will sway, twist, or bend, which results in less precise pixel analyses for these ROI. In contrast, elastomers tested at lower rates are more fixed in position and do not move after they break. While not wishing to be bound by theory, it is believed that this variation may be attributed to: differences in the extent of SIC, and the \dot{ε}-dependence is consistent with DSC results discussed in Section 1.4; slowly stretching results in more extensive SIC, and correspondingly, that there are fewer chains in amorphous regions that may otherwise enable relaxation after failure.

### 1.9. Stress Relaxation Tests

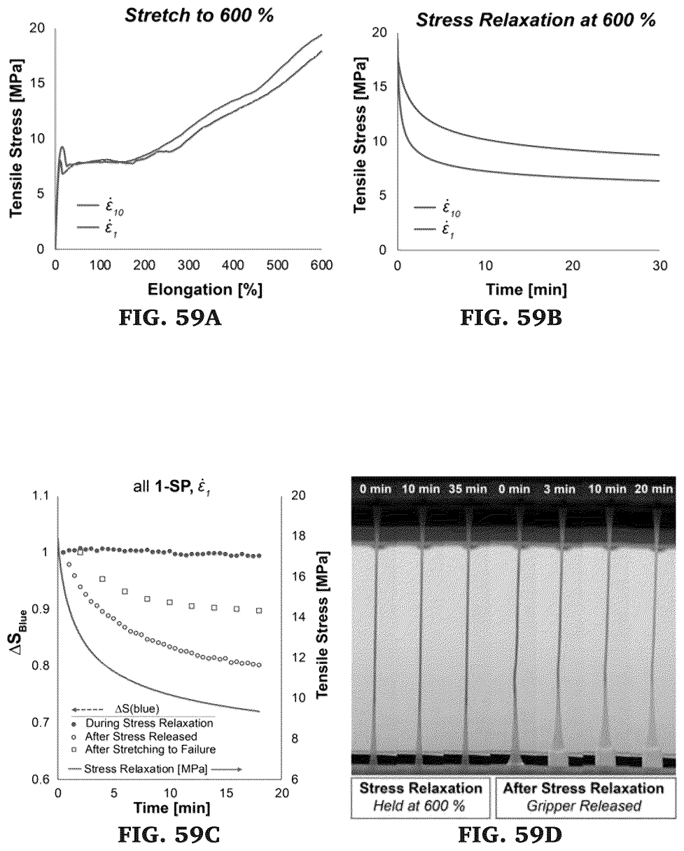

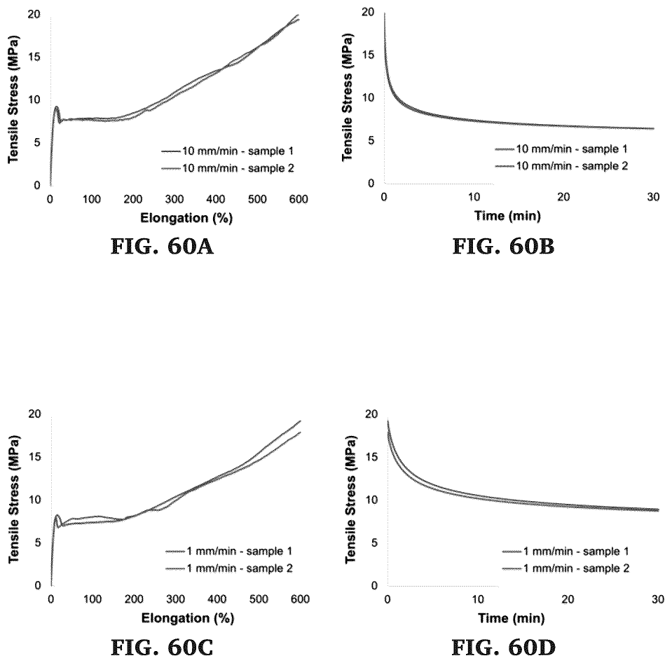

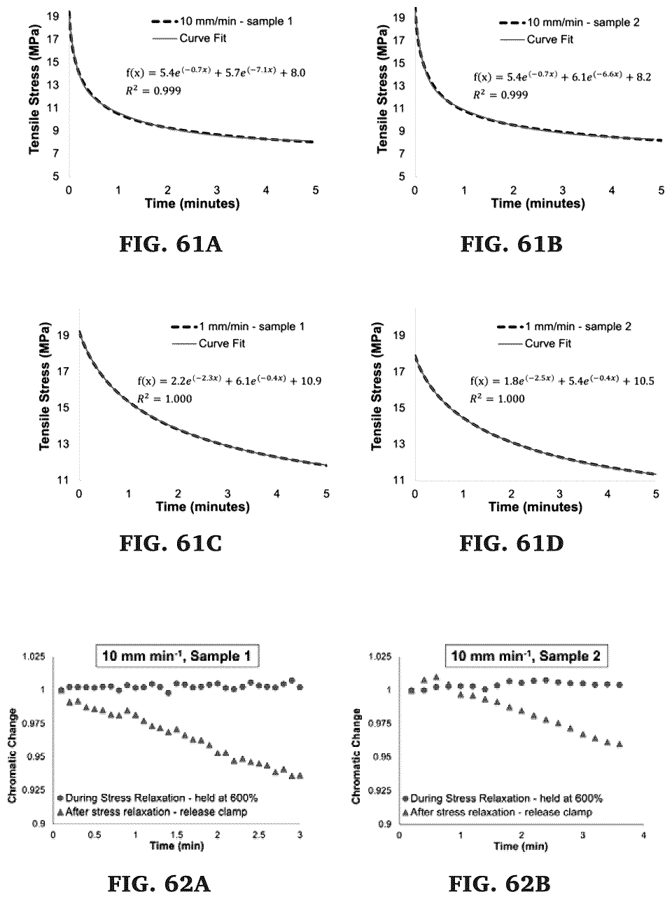

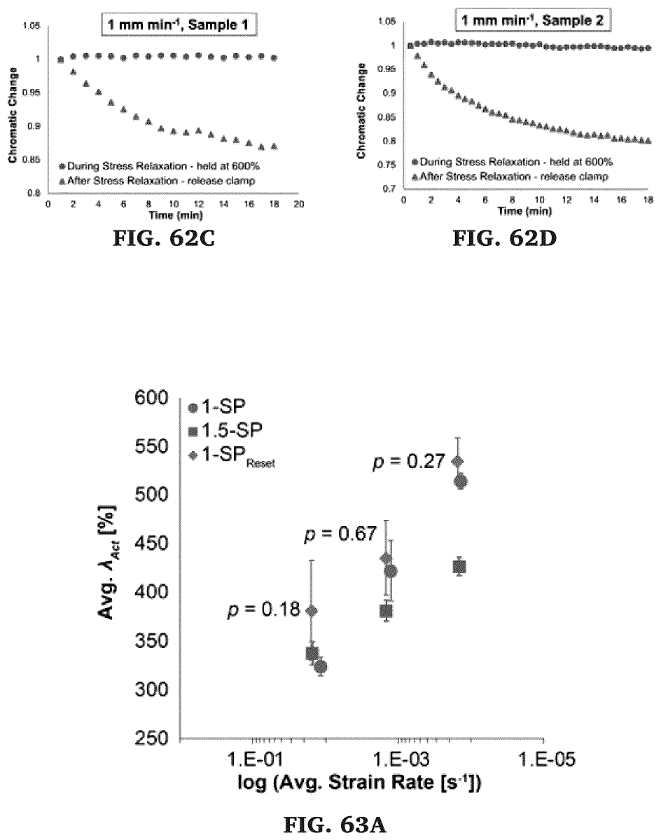

Stress Relaxation (SR) tests were completed for 1-SP to determine the \dot{ε}-dependence of SR. Samples of 1-SP were stretched at \dot{ε}₁₀ and \dot{ε}₁ until reaching λ=600%, then fixed in that position, and the subsequent SR kinetics were measured (FIGS. 59A-B). 600% was chosen because it is within 80% of the average elongation at break for 1-SP; all tests were completed in duplicate, and all results are included in FIGS. 60-62. Images were captured concurrently to quantify any change in ΔS_{blue}. After samples relaxed to stress levels lower than those measured at the start of SH (between 7-9 MPa), the bottom gripper of the Instron was released. Subsequently, SP decay kinetics were measured for the same ROI (size and position) used for images from SR. SR kinetics were fit to a 2-phase exponential decay curve according to Equation (4):

σ=σ_{f}*e^{(-k}^{_{f}}^{t)}+σₛ*e^{(-k}^{ₛ}^{t)}+σ_{∞} (4)

Where σ is stress as measured, σ_{∞}, σ_{f}, and σₛ are constants, and k_{f} and kₛ are rate constants of the fast and slow phase, respectively.

An example data set of SR kinetics is pictured in FIG. 59C-D. SR kinetics indeed display \dot{ε}-dependence; when fit according to Equation (3) in Matlab, \dot{ε}₁₀-tested samples display an average k_{f} of 6.840±0.305 min⁻¹ and kₛ of 0.694±0.032 min⁻¹. In contrast, the \dot{ε}₁-tested samples displayed slower SR kinetics, with average k_{f}=2.333±0.252 min⁻¹ and kₛ=0.373±0.008 min⁻¹. Additionally, there is a greater magnitude of SR for \dot{ε}₁₀-tested samples, in comparison to \dot{ε}₁. These results are indeed consistent with DSC measurements of \dot{ε}-dependent SIC in 0-SP, as detailed in Section 1.4. For all samples, there is no significant change in mechanochromism during SR, as ΔS_{blue} remains mostly constant (FIG. 59C). After tensile stress was relaxed to approximately 7-9 MPa, the bottom sample gripper was released, at which point ΔS_{blue} began to decrease exponentially, with kinetics like those measured after failure.

### 1.10 General Procedure for Strain Rate-Dependent SP Recovery Kinetics

SP decay kinetics are calculated from pixel data in the same procedure as for SP activation during tensile tests. 4 ROI of identical size and shape were used for every polymer sample. Chromatic change is calculated as detailed above in sections 1.5 and 1.6.

Chromatic change data were imported into Matlab, then fitted with a 1-phase exponential decay curve, according to Equation (7), where a and c are constants, and k is the decay rate constant:

ΔS_{blue}=a*e^{-k*t}+c (7)

This procedure was repeated for all ROI and all samples, then rate constants were averaged from n=12 different kinetics fittings.

### 1.11. Recyclability

Previous publications have reported that linear stereoelastomers display shape memory behavior that is comparable to that observed in chemically cross-linked networks. (See e.g., J. C. Worch, A. C. Weems, J. Yu, M. C. Arno, T. R. Wilks, R. T. R. Huckstepp, R. K. O'Reilly, M. L. Becker, A. P. Dove, Nat Commun 2020, 11, 3250; M. B. Wandel, C. A. Bell, J. Yu, M. C. Arno, N. Z. Dreger, Y. H. Hsu, A. Pitto-Barry, J. C. Worch, A. P. Dove, M. L. Becker, Nat Commun 2021, 12, 446, the disclosures of which are incorporated herein by reference in their entirety). Within these non-cross-linked thermoplastics, shape memory is attributed to robust chain entanglements, as well as the temperature-dependent formation and melting of crystalline domains. This process is observed readily in the DSC experiments of 0-SP: all strain-induced crystallites are melted during the 1^{st} heating cycle, thereby erasing the SIC from the thermal history. As such, it is of interest to investigate in the interplay of shape memory and mechanochromism in recycled 1-SP samples, referenced herein as 1-SP_{Reset}.

Samples of 1-SP that had been stretched to failure (at all applied ε) were combined, then melt-pressed into a new film using the same procedure described in Section 1.2. The reset behavior in 1-SP_{Reset} is linked directly to this process. First, the used 1-SP samples were heated above Tₘ, which melted the strain-induced crystallites. Subsequently, samples were cooled to room temperature while under confinement, producing a film with properties comparable to the original 1-SP. DSC measurements confirm that 1-SP Reset possesses similar thermal properties to 1-SP (Table 5) (Appendix), as there was no significant difference between samples for either heating cycles.

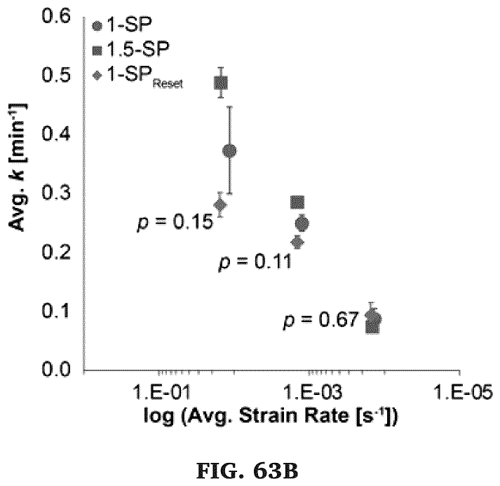

SP_{Reset} was subjected to uniaxial deformation under all three strain rates. The mechanical properties of SP_{Reset} were found to be consistent with those of the original 1-SP material (Table 3) (Appendix). Image analysis confirms that mechanochromism also follows the same trends (see, FIGS. 63A-B). Compared to 1-SP, the extrapolated λ_{Act} for SP_{Reset} occurs at statistically indistinguishable elongations (p-values>0.1, for all rates). This trend is observed similarly in the calculated SP decay constants. Calculations of k for \dot{ε}₁₀, \dot{ε}₁, and \dot{ε}_{0.1} tests were approximately 25, 13, and 8% lower than the respective values obtained from the parent 1-SP.

### 2. Discussion

As evidenced most concisely in FIG. 5D, the \dot{ε}-dependence of mechanochromism, mechanical properties, and SIC are correlated highly. Below, we summarize and discuss these correlations and their potential consequences.

### 2.1. Strain Rate-Dependent SH, SIC, and Mechanochromism

The \dot{ε}-dependence exhibited by these elastomers confirms that viscoelastic relaxation from crystallite dissociation and formation plays a significant role in material properties. More specifically, there is some degree of relaxation within elastomer amorphous domains that enables this apparent dependent on \dot{ε}. A 10-fold increase in \dot{ε} is correlated with a corresponding change in elastic moduli, SH behavior, extent of SIC, as well as several measures of mechanochromism. Importantly, the extrapolated λ_{Act} also exhibits \dot{ε}-dependence; for any 10-fold decrease in \dot{ε}, there are corresponding delays in SP activation of up to 100% additional λ. This significant delay is almost an order of magnitude greater than other reports of \dot{ε}-dependent mechanophore activation for comparable changes in \dot{ε}.

### 2.2. Strain Rate-Dependent SIC Enables Force-Trapping of Activated Mechanophore

The extent of SIC, which is dependent on the applied \dot{ε}, correlates strongly with the decay kinetics of the activated mechanophore. Specifically, stretching slowly correlates with more extensive SIC, presumably because, relative to the applied \dot{ε}, chains have more time to move into optimal orientations for enhanced crystal packing. This logic has been used to understand why low \dot{ε} results in greater mechanophore activation (i.e., greater color change); there is a finite amount of time needed for the mechanophores to move into optimal orientation, relative to the strain field, for mechanochemical activation. Because SIC is similarly dependent on chain orientation, it follows that there is a finite amount of time required for chains to move into optimal alignment for crystal formation. Consequently, stretching slowly results in more extensive SIC growth, which in turn inhibits SP relaxation after the (macroscopic) release of force. Stress relaxation tests also support these conclusions because stress relaxation kinetics are similarly \dot{ε}-dependent, and thus correlated with SIC.

To the best of our knowledge, this represents the longest reported activation lifetime of this spiropyran derivative and decay times are comparable to resonance-stabilized, nitro-substituted SPs. These rate-dependent decay kinetics may benefit several potential applications. For example, the tunable reversibility achieved through the extent of SIC may enable novel tamper reporting technologies for flexible electronics. Additionally, SIC may provide a platform for researching mechanophore properties, especially for those with more ephemeral activation states, as is the case with the SP mechanophore used herein.

### 2.3. Multi-Regime ΔS_{blue} Indicates SP is Activated in Multiple Morphological Domains

The multi-regime mechanochromism observed in these elastomers may represent sequential SP activation within different morphological domains. These regimes are most clearly delineated by the boundary between the tensile SH regimes. Since later SH is accompanied by an increase in scattering, these regimes are likely dominated by SIC processes; conversely, early SH corresponds to chain overstretching in amorphous domains. More specifically, during early SH there is a competition between the disruption of interchain van der Waals forces (within both crystalline and amorphous domains), and the activation of SP. This competition is consistent with the macroscopic multi-regime SP response; first, there is a slow development of blue color (van der Waal disruption-dominant), which is then followed by a dramatic chromatic change with strain (SP activation-dominant).

As these chains are extended further (to elongations associated with late SH, where λ>λ_{Act}), it's possible that some SP-activated chains undergo a phase change to crystalline during SIC. This is most evidenced FIGS. 19A-B and 20A-B; in late SH (i.e., λ>λ_{Act}), all elastomers display an increase in the slope of ΔS_{blue}, which is accompanied by PW effects. It is unclear if there is any SP activation within (already) crystalline domains; the relative energy barrier of any possible disruption to crystal packing may not be substantial enough to enable SP activation.

Additional details cannot be inferred from the observed behaviors. For example, it is not known if SP is heterogeneously dispersed within the crystalline and amorphous domains. If SP content is biased towards one domain over another, this could affect any future quantification and analysis of the source of each of the mechanochromism regimes. Ongoing work will focus on elucidating the mechanisms underlying this multi-regime mechanochromism.

The implications of these results include the likelihood that mechanophore activation directly reports the onset of SIC within stereoelastomer materials. Mechanophores offer a macroscopic visualization of molecular-level stresses, so their application to SIC monitoring could bridge insights from current macro- and microscopic methods of monitoring SIC. In practice, this means that SIC could be monitored by not only visible eye, but also programmable, quantifiable methods based on principles of absorbance and fluorescence. Such technology could even provide in vivo, real-time monitoring of SIC within biomedical implants, with the aid of embedded nano sensors. (See, e.g., T. R. Ray, J. Choi, A. J. Bandodkar, S. Krishnan, P. Gutruf, L. Tian, R. Ghaffari, J. A. Rogers, Chem. Rev. 2019, 119, 5461; O. Rifaie-Graham, E. A. Apebende, L. K. Bast, N. Bruns, Adv. Mater. 2018, 30, e1705483, the disclosures of which are incorporated herein by reference in their entirety.

### 2.4. Recyclability Supports Potential for Application to Shape-Memory Systems

Aside from the obvious environmental and economic benefits associated with recyclable plastics, the studies of recycled 1-SP_{Reset} indicate that stereoelastomers are a new platform to incorporate mechanochromism within shape-memory materials, which rely on temperature-dependent erasure of material thermal history. Because the morphological domains result from stereochemistry of the backbone alkene, one can generally assume there are no localized polarity differences between hard and soft and (i.e., cis and trans) regions which would otherwise bias the mechanophore' s force-coupled equilibrium.

### 3. Conclusion

In summary, the interplay of mechanochromism and SIC within semi-crystalline SP-doped thiol-yne stereoelastomers has been investigated thoroughly. Material properties are influenced heavily by viscoelastic effects due to chain entanglement, as evidenced by their \dot{ε}-dependent moduli, SH and SIC behaviors. Mechanochromism is also \dot{ε}-dependent, such that SP activation depends significantly on the applied \dot{ε}. Furthermore, SP relaxation is correlated heavily with the extent of SIC, resulting in the longest reported activation lifetime for this SP derivative, to date. Importantly, these elastomers also display multi-regime mechanochromism, and the transitions between these regimes correlate with the onset of SIC. While these results are not yet quantitative, these data indicate that mechanophore activation may represent a new strategy for real-time monitoring of material deformation and SIC. Ongoing research will work to quantify the precise nature of this multi-regime relationship in the hope to further extend these studies to thiol-yne-derived stereoelastomers of different cis content, and thus different proclivities towards SIC.

### EXAMPLES

The following examples are offered to more fully illustrate the invention but are not to be construed as limiting the scope thereof. Further, while some of examples may include conclusions about the way the invention may function, the inventor does not intend to be bound by those conclusions but put them forth only as possible explanations. Moreover, unless noted by use of past tense, presentation of an example does not imply that an experiment or procedure was, or was not, conducted, or that results were, or were not actually obtained. Efforts have been made to ensure accuracy with respect to numbers used (e.g., amounts, temperature), but some experimental errors and deviations may be present. Unless indicated otherwise, parts are parts by weight, molecular weight is weight average molecular weight, temperature is in degrees Centigrade, and pressure is at or near atmospheric.

### Materials

1,8-diazabicyclo[5.4.0]undec-7-ene (DBU), 4-(dimethylamino)pyridine (DMAP), N,N′-diisopropylcarbodiimide (DIC), 6-bromohexanoic acid, propiolic acid, 1,6-hexanedithiol, 1,3-propanediol, butylated hydroxytoluene (BHT), sulfuric acid (H₂SO₄), sodium hydroxide (NaOH), sodium bicarbonate (NaHCO₃), ammonium chloride (NH₄Cl), sodium sulfate (Na₂SO₄), aluminum oxide (activated, neutral), silica gel, sodium chloride (NaCl), anhydrous dichloromethane (DCM), ethyl acetate (EtOAc), chloroform (CHCl₃), N,N-dimethylformamide (DMF), methanol (MeOH), hexane, diethyl ether (Et₂O), chloroform-d (CDCl₃) and methanol-d₄ (CD₃OD) were purchased from Sigma-Aldrich (St. Louis, MO). All commercial reagents and solvents were used as received without further purification except 1,6-hexanedithiol, which was purified via vacuum distillation according to analogous procedures.

### Characterization

¹H NMR (500 MHz) and ¹³C NMR (126 MHz) spectra were obtained using a Bruker 500 MHz NMR spectrometer operated at room temperature (RT). All chemical shifts are reported in ppm (δ) and referenced to the chemical shifts of residual solvent resonances (CDCl₃ ¹H: δ=7.26 ppm, ¹³C: δ=77.16 ppm; CD₃OD ¹H: δ=4.87, 3.31 ppm, ¹³C: δ=49.00 ppm). ¹H resonances are reported as chemical shift, multiplicity, coupling constant if applicable, and relative integral. Multiplicities are reported as: singlet (s), doublet (d), doublet of doublets (dd), doublet of triplets (td), quartet (q), multiplet (m), or broad (br). Coupling constants (J) are reported in Hz.

High-resolution mass spectra were collected on an Agilent LCMS-TOF-DART at Duke University's Mass Spectrometry Facility. Size exclusion chromatography (SEC) was performed on polymer samples using an EcoSEC HLC-8320 GPC (Tosoh Bioscience LLC, King of Prussia, PA) equipped with a TSKgel GMH_{HR}-M mixed bed column and refractive index (RI) detector. Molecular masses were calculated using a calibration curve determined from polystyrene standards (PStQuick MP-M standards, Tosoh Bioscience, LLC) with CHCl₃ as eluent flowing at 1.0 mL·min⁻¹ at 323K, and a sample concentration of 3 mg·mL⁻¹.

Differential scanning calorimetry (DSC) was performed using a TA Instruments Q200 DSC (TA Instruments—Waters L.L.C., New Castle, DE) on sample sizes between 5-10 mg using temperature ramps for heating of 10° C.·min⁻¹ and a cooling rate of 10° C.·min⁻¹ from −20° C. to 140° C. The glass transition temperature (T_{g}), crystallization temperature (T_{c}), melting point (Tₘ), and enthalpy of melting (ΔHₘ) were determined from the second heating cycle of DSC, unless otherwise noted.

### Example 1

### Synthesis of 6-Bromohexanoic Anhydride (1)

6-bromohexanoic anhydride 1 was synthesized according to a modified previously published procedure. (See, R. K. Pathak, S. Dhar, Chem. Eur. J. 2016, 22, 3029, the disclosure of which is incorporated herein by reference in ite entirety.) A 3 neck 500 mL round bottom flask outfitted with stir bar and addition funnel were flame dried then cooled to ambient temperature under ambient air. 6-bromohexanoic acid (16.02 g, 82.22 mmol, 1 eq.) was dissolved in anhydrous DCM (225 mL) in the flask, then cooled to 0° C. for 30 min. A solution of N,N′-diisopropylcarbodiimide (DIC; 6.21 g, 49.3 mmol, 0.6 eq.) in anhydrous DCM (100 mL) was added to the addition funnel, then the solution was added to the main reaction flask drop-wise over 30 min. The reaction was warmed to ambient temperature and stirred under N₂ overnight. The resultant precipitate was removed using filtration, then the crude product was concentrated, redissolved in ethyl acetate, then filtered once again. Evaporation of the solvent gave 1 as a transparent oil in quantitative yield. The product was used immediately in the following reaction without further characterization.

### Example 2

### Synthesis of 2-(8-((6-bromohexanoyl)oxy)-3′,3′-dimethylspiro [chromene-2,2′-indolin]-1′-yl) ethyl 6-bromohexanoate (3)

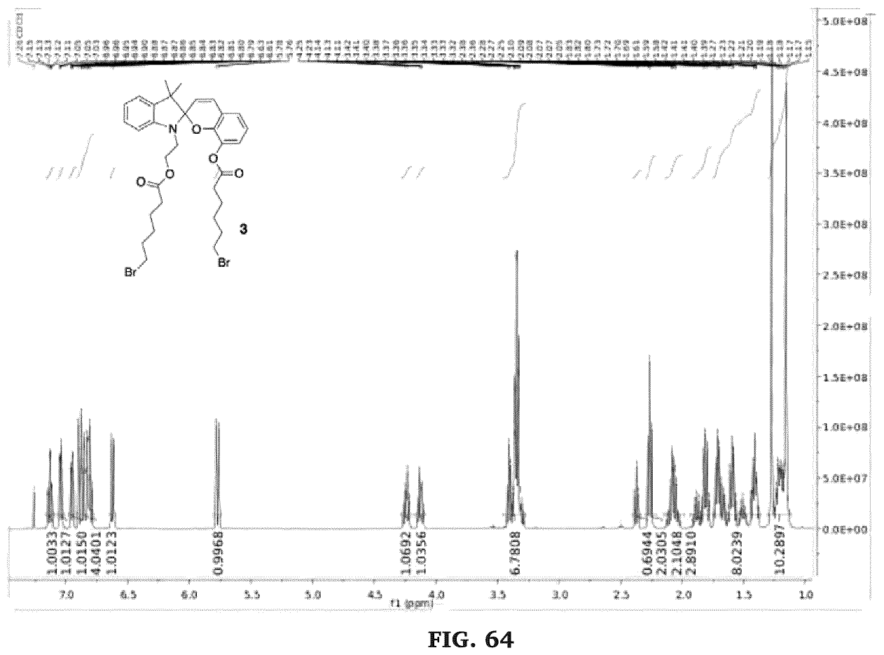

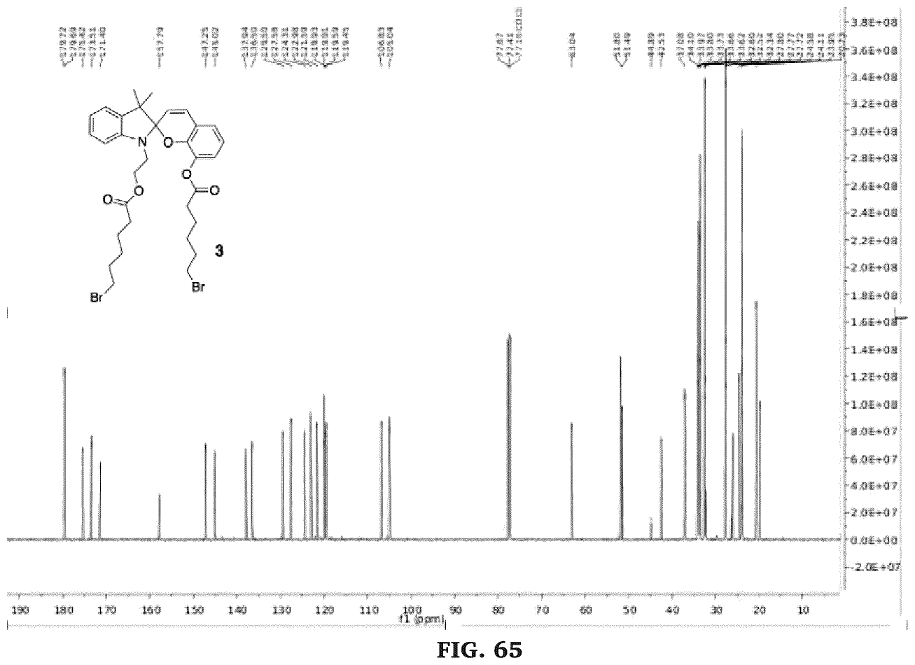

2-(8-((6-bromohexanoyl)oxy)-3′,3′-dimethylspiro [chromene-2,2′-indolin]-1′-yl) ethyl 6-bromohexanoate 3 was synthesized as follows. SP diol 2 (4.00 g, 12.4 mmol, 1 eq.) and N,N′-dimethylamino pyridine (DMAP; 0.15 g, 1.2 mmol, 0.01 eq.) were dissolved in anhydrous DCM (25 mL). 6-bromohexanoic anhydride 1 (9.91 g, 1.24 mmol, 2.15 eq.) was added drop-wise over 5 min, turning the solution from dark purple to a light red/brown. The reaction was stirred overnight at ambient temperature, then concentrated. The reaction was re-dissolved in ethyl acetate then washed with water, sodium bicarbonate, and brine before drying on sodium sulfate. The solution was concentrated onto neutral aluminum oxide and purified via column chromatography (hexane/ethyl acetate) to yield 3 as a reddish (68%). ¹H NMR (500 MHz, CDCl₃) δ7.13 (td, J=7.7, 1.2 Hz, 1H), 7.07-7.01 (m, 1H), 6.95 (dd, J=7.6, 1.6 Hz, 1H), 6.91-6.77 (m, 4H), 6.62 (d, J=7.8 Hz, 1H), 5.77 (d, J=10.3 Hz, 1H), 4.24 (dt, J=11.1, 6.4 Hz, 1H), 4.13 (dt, J=11.6, 6.1 Hz, 1H), 3.46-3.27 (m, 7H), 2.38 (t, J=7.4 Hz, 1H), 2.27 (t, J=7.4 Hz, 2H), 2.15-1.99 (m, 2H), 1.85 (dp, J=35.2, 6.9 Hz, 3H), 1.75-1.36 (m, 8H), 1.30-1.11 (m, 10H). (See, FIG. 64) ¹³C NMR (126 MHz, CDCl₃) δ179.46, 179.44, 175.17, 173.25, 171.14, 157.53, 146.99, 144.76, 137.68, 136.25, 129.25, 127.33, 124.06, 122.73, 121.33, 119.67, 119.65, 119.34, 119.19, 106.57, 104.78, 77.41, 77.16, 76.90, 62.78, 51.55, 51.23, 44.63, 42.28, 36.83, 33.85, 33.71, 33.55, 33.47, 33.41, 33.37, 32.35, 32.26, 32.10, 32.08, 27.54, 27.52, 27.47, 26.20, 25.79, 24.32, 23.86, 23.82, 23.70, 23.60, 20.48, 19.42. (See. FIG. 65) High Resolution Mass Spectroscopy (HRMS) (m/z): [M+H]⁺ Calculated for C₃₂H₃₉Br₂NO₅ 678.4; observed 678.2. (See also, Scheme 4, above).

### Example 3

### Synthesis of 3′,3′-dimethyl-1′-(2-((6-(propioloyloxy)hexanoyl)oxy)ethyl) spiro[chromene-2,2′-indolin]-8-yl 6-(propioloyloxy)hexanoate (4)

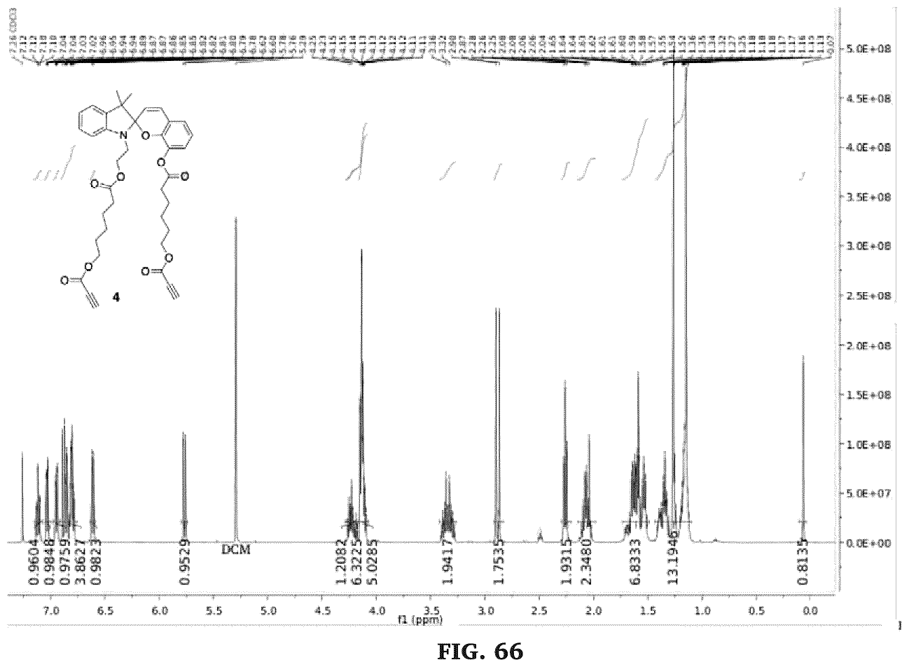

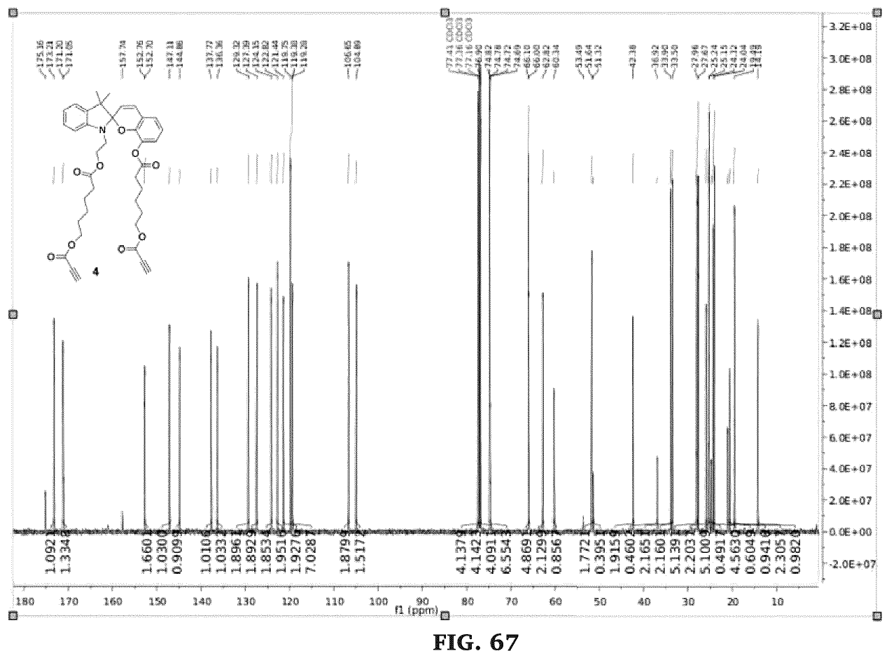

3′,3′-dimethyl-1′-(2((6-(propioloyloxy)hexanoyl)oxy)ethyl) spiro[chromene-2,2′-indolin]-8-yl 6-(propioloyloxy)hexanoate 4 was synthesized as follows. A 2-neck 100 mL round bottom flask with magnetic stir-bar was flame dried, then cooled to ambient temperature under ambient air, in a dark fume hood. 3 (2.41 g, 3.56 mmol, 1 eq.) was added quantitatively with anhydrous DMF (30 mL). Sodium propiolate (3.30 g, 35.6 mmol, 10 eq.) was added to the flask; please note that sodium propiolate decomposes in ambient light and should only be handled in the dark. The reaction flask was then sealed and heated to 80° C. with stirring. After 2 h, the reaction was cooled to ambient temperature and transferred to a 500 mL beaker with saturated NH₄Cl (200 mL) and stirred for 10 min. The crude product was extracted with ethyl acetate (200 mL), and washed with DI water 5×, brine 2×, then DI water 1×, before drying over Na₂SO₄. The crude product was then filtered, concentrated onto silica gel, and dried under vacuum for 10 h. The product was purified with flash chromatography, 0% to 30% ethyl acetate in hexane, yielding 4 as a light blue oil (36%). ¹H NMR (500 MHz, CDCl₃) δ7 δ7.12 (td, J=7.7, 1.3 Hz, 1H), 7.03 (dd, J=7.4, 1.2 Hz, 1H), 6.95 (dd, J=7.5, 1.6 Hz, 1H), 6.91-6.76 (m, 4H), 6.61 (d, J=7.8 Hz, 1H), 5.77 (d, J=10.3 Hz, 1H), 4.28-4.08 (m, 6H), 3.34 (qt, J=15.2, 6.3 Hz, 2H), 2.88 (d, J=15.6 Hz, 2H), 2.26 (t, J=7.4 Hz, 2H), 2.15-1.98 (m, 2H), 1.73-1.49 (m, 7H), 1.43-1.10 (m, 13H), 0.07 (s, 1H). (See, FIG. 66) ¹³C NMR (126 MHz, CDCl₃) δ175.16, 173.21, 171.20, 171.05, 157.74, 152.76, 152.70, 147.11, 144.86, 137.77, 136.36, 129.32, 127.39, 124.15, 122.82, 121.44, 119.75, 119.38, 119.28, 106.65, 104.89, 77.41, 77.36, 77.16, 76.90, 74.82, 74.78, 74.72, 74.69, 66.10, 66.00, 62.82, 60.34, 53.49, 51.64, 51.32, 42.38, 36.92, 33.90, 33.50, 28.11, 27.96, 27.82, 27.67, 25.85, 25.34, 25.24, 25.15, 24.79, 24.32, 24.04, 21.01, 20.54, 19.49, 14.19. (See, FIG. 67). HRMS (m/z): [M+H]⁺ Calculated for C₃₈H₄₁NO₉ 656.7; observed 656.3. (See also, Scheme 4, above).

### Example 4

### General Procedure for Thiol-Yne Step Growth Polymerization

Commercially available 1,6-hexanedithiol 6 (2.51 g, 16.7 mmol, 1 eq.), propane-1,3-diyl dipropiolate 5 (3.00 g, 16.6 mmol, 0.995 eq.), and SP monomer (3′,3′-dimethyl-1′-(2-((6-(propioloyloxy)hexanoyl)oxy)ethyl)spiro[chromene-2,2′-indolin]-8-yl 6-(propioloyl oxy)hexanoate) 4 (0.05 g, 0.08 mmol, 0.005 eq.) were added to a 100 mL round bottom flask with CHCl₃ (35 mL). The solution was then cooled to −15° C. with stirring for 20 min. 1,8-Diazabicyclo[5.4.0]undec-7-ene (DBU; 25.0 μL, 0.167 mmol, 0.01 eq.) was diluted with CHCl₃ (3 mL) before adding to the reaction dropwise over 20 min. Notably, the addition of DBU caused the solvent to bubble due to an exothermic reaction. After 10 min, the reaction was allowed to warm to room temperature. After an additional 2 h, several drops of 5 were added into reaction solution with CHCl₃ (5 mL) to endcap any remaining thiol groups. After another 0.5 h, butylated hydroxytoluene (BHT) (˜200 mg) was added to prevent any cross-linking. The polymer was then precipitated into Et₂O (500 mL) and collected by decanting the supernatant. The polymer was then dried under high vacuum at ambient temperature for 24 h to obtain the pale-yellow polymer. Size exclusion chromatography (SEC; CHCl₃) Mₙ=16.4 kDa, M_{w}=50.4 kDa, [symbol] _{M}=3.1. (See, FIGS. 2A-D). ¹H NMR (CDCl₃, 500 MHz) % cis ˜79-80%, with 99% incorporation of SP monomer 4. (See also, Scheme 5, above).

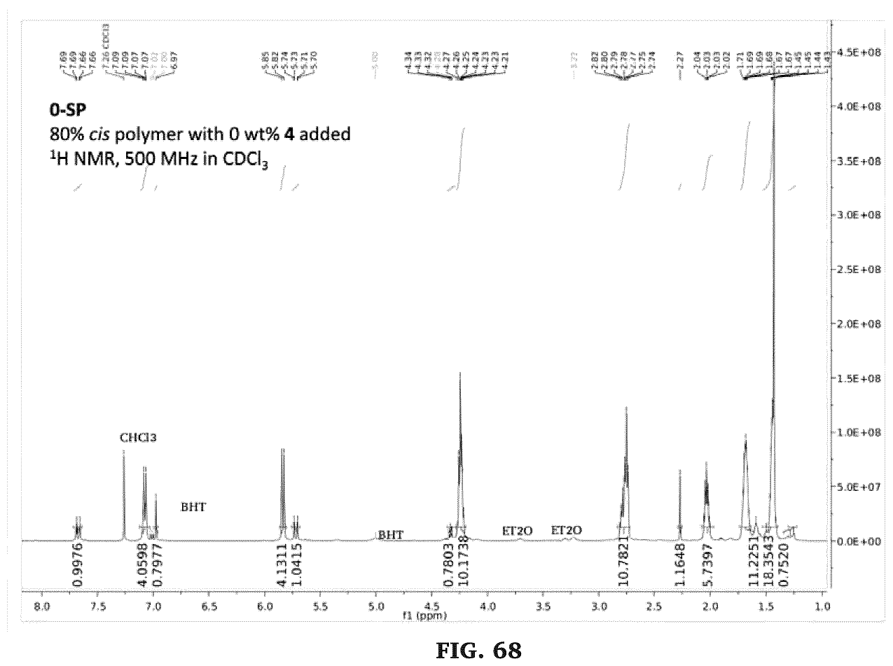

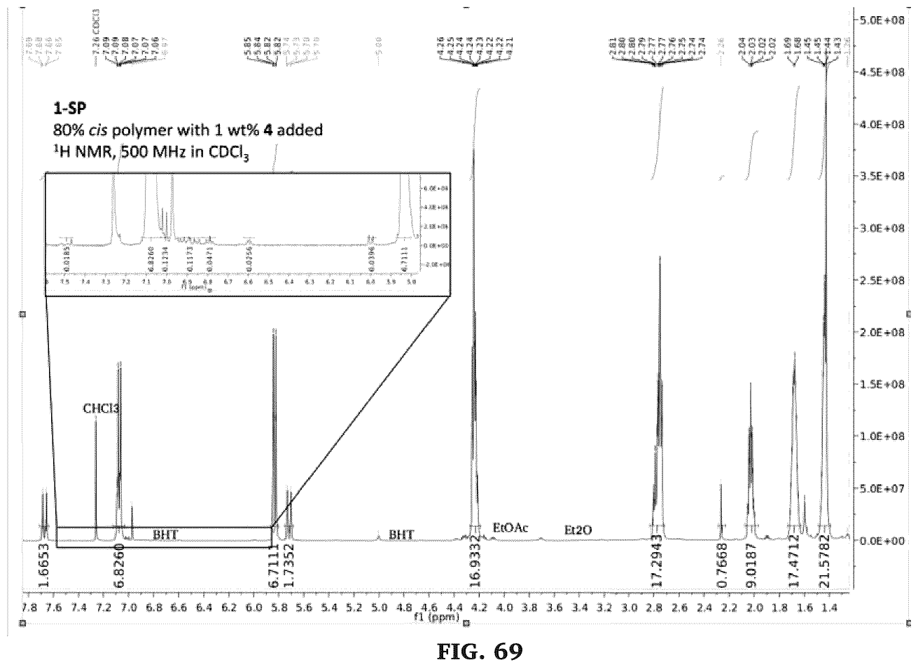

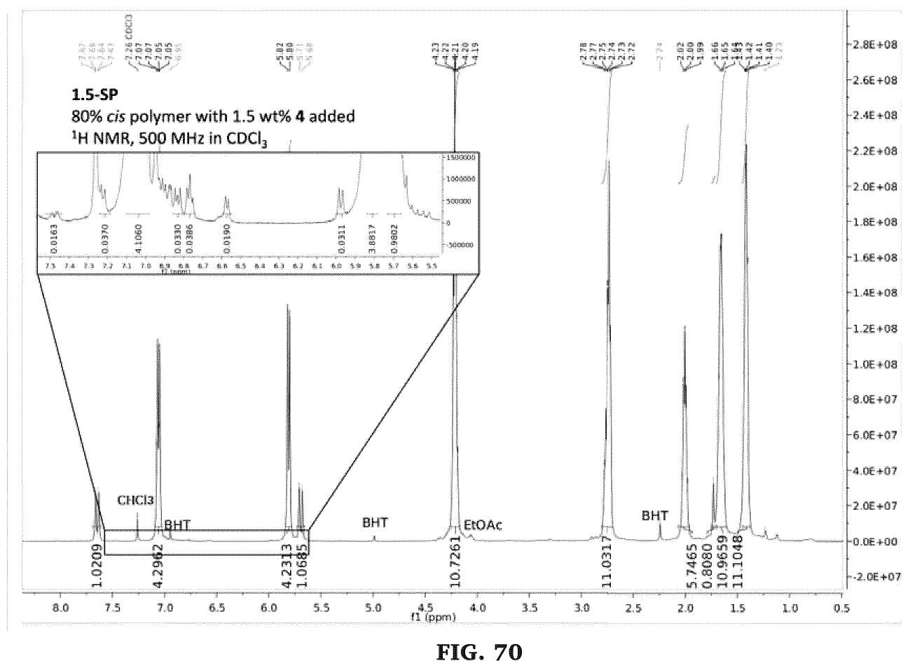

A ¹H NMR (500 MHz, CDCl₃) for a thiol-yne step growth polymer made using 80% cis polymer with no (0 wt. %) 3′,3′-dimethyl-1′-(2-((6-(propioloyloxy)hexanoyl)oxy)ethyl) spiro[chromene-2,2′-indolin]-8-yl 6-(propioloyloxy)hexanoate (polymer 4) (0-SP) is shown in FIG. 68. A ¹H NMR (500 MHz, CDCl₃) for a thiol-yne step growth polymer made using 80% cis polymer with 1 wt. %) polymer 4 (1-SP) is shown in FIG. 69. A ¹H NMR (500 MHz, CDCl₃) for a thiol-yne step growth polymer made using 80% cis polymer with 1 wt. % polymer 4 (1.5-SP) is shown in FIG. 70.

### Example 5

### Statistical Analysis

Mechanical data were obtained from tensile tests using methods detailed in Section 1.3; all mechanical data are reported as an average ±1 standard deviation for n=3 independent trials. Thermal data were obtained from DSC using methods detailed in Section 1.4, and they are reported as an average of n=2 independent trials. Chromaticity data were obtained from pixel analyses of digital images, using methods detailed in Section 1.5. λ_{Act} extrapolations were obtained using methods detailed in Section 1.7, and data are presented as an average ±1 standard deviation for n=3 independent trials. SP recovery kinetics data were obtained using methods detailed in Section 1.8. SP Recovery kinetic constants, k, were obtained by fitting data to an exponential decay (Equation (3) in Matlab, and they are reported as average ±1 standard deviation for n=12 ROI (3 independent samples; 4 independent ROI per sample). Stress relaxation kinetics constants, k_{f} and kₛ, were obtained by fitting tensile data to a two-phase exponential decay (Equation (4) in Matlab, and they are reported as an average of n=2 independent experiments. To compare the mechanochromism properties of 1-SP_{Reset} and 1-SP, average values for λ_{Act} and k were compared for each applied \dot{ε}. P-values (2-tailed Student's T-test) were calculated using Excel.

### Example 6

### Mechanical Property Measurements

### Preparation of Tensile Bar Samples

Films of each polymer were fabricated using a Bubble Magic Pneumatic melt-compression machine. The machine was pre-heated to 10° C. above Tₘ, then polymer was added into a 50×50×1.00-mm mold and placed on the compression machine under ambient air and pressure. After 15 min of melting, the polymer was compressed under 100 psi of pressure for 20 minutes. After that, the polymer was cooled to RT under 100 psi of pressure to prevent wrinkling of the film's surface. The films were visually inspected to ensure that no bubbles were present in the films, then dumbbell-shaped samples were cut using a custom ASTM Die D-638 Type V. The dimensions of the neck of the specimens were 9.53 mm in length, 1.50 mm in width, and 0.75-1.00 mm in thickness.

### Uniaxial Tensile Tests at Different Strain Rates

Tensile tests were conducted at strain rates of 10 mm/min, 1 mm/min, and 0.1 mm/min, using dumbbell-shaped samples that were prepared as described above. All tests were done in triplicate to ensure reliability. Tensile tests were carried out using an Instron 5965 Universal Testing Machine at room temperature (25±1° C.) with a 1 kN load cell. Gauge lengths, crosshead speeds, and deformation rates are listed in Table 4 (Appendix).

### Example 7

### Digital Image Analysis

### Image Acquisition and Processing

Digital images were captured using a Canon EOS Rebel™ T7 with a Canon EF-S 18-55 mm f/3.5-5.6 IS SLR lens. Ambient fluorescent room lighting was used without flash, with exposure settings of ⅙ sec, f/4.5, ISO 100. Focus was controlled manually and all camera settings within the data set were kept the same. Images were captured at regular intervals using a JCC Intervalometer remote-control timer and shutter release; these intervals corresponded to the time needed for either 1 mm or 2 mm displacement of the Instron crosshead beam. By synchronizing the intervalometer with the start of the tensile test, the corresponding nominal strain associated with each image was obtained. A color-neutral target (Opteka™ Digital Color & White Balance Card) was placed in the background to aid in quantitative analysis. A white card possesses spectral neutrality and uniformity under all sources of illumination and allows for all images to be calibrated and corrected to a known standard. Raw images available upon request.

Images were captured in a .CR2 raw format to prevent automatic corrections and to retain all information within the image so that RGB intensities could be measured quantitatively. Raw images were imported into Adobe Lightroom as .CR2 files, white balanced to a neutral target, and the same edits were applied to every photo within the image sequence that makes up the experiment (settings→sync). Images were then exported as .TIFF files for use in Fiji (ImageJ) without any further modification or compression.

### Obtaining Pixel Data in Fiji

The procedure for obtaining mean pixel data for the RGB color channels in Fiji is analogous to that used previously in the literature. (See, e.g., M. H. Barbee, K. Mondal, J. Z. Deng, V. Bharambe, T. V. Neumann, J. J. Adams, N. Boechler, M. D. Dickey, S. L. Craig, ACS Appl. Mater. Interfaces 2018, 10, 29918, the disclosure of which is incorporated herein by reference in its entirety).TIFF files exported from lightroom were imported into Fiji as an image sequence (File→Import→Image Sequence). The stack is converted to a hyperstack (Image→Hyperstacks→Stack to Hyperstack) with 3 channels red, green, and blue. A rectangular region of interest (ROI) is added to the ROI manager (Analyze→Tools→ROI Manager), then the mean pixel intensity (MPI) is measured for every channel and image in the stack. This was repeated for every image in the stack so that the ROI is within the center of the tensile sample for each image, taking extra care to avoid overlap with sample edges.

### Calculation of Chromatic Change from Pixel Data

An example of the MPI raw data for the three RGB channels is shown in FIG. 5A. For all tests, the MPI data initially increases dramatically across all color channels, until the sample reaches ˜50% strain. This initial increase is attributed to a shadow effect from the top of the sample gripper; as the gripper moves upward, this allows more photons to reach the camera, resulting in greater pixel intensities. For this reason, pixel data for the first 0-50% strain were not used for any quantitative analyses or conclusions on SP activation.

The Total Chromaticity, Sₜₒₜₐₗ, was calculated for each image from the sum of the color channel MPIs, R, G, and B, (Equation (5)) as described by Creton et al.:

Sₜₒₜₐₗ=R+G+B (5)

(See, Y. Chen, C. J. Yeh, Y. Qi, R. Long, C. Creton, Sci. Adv. 2020, 6, eaaz5093, the disclosure of which is incorporated herein by reference in its entirety). (See, FIGS. 11A-C, 12A-C). The chromaticity of each color channel, Sᵣₐₜᵢₒ, was defined as the ratio of their respective MPIs to Sₜₒₜₐₗ, as shown in FIG. 11B and Equation (6):

$${S}_{ratio} = \frac{R , G or B}{{S}_{total}} \qquad ( 6 )$$

All data sets were then normalized to their Sᵣₐₜᵢₒ values at 0% tensile strain, which allowed for quantification of the Chromatic Change, ΔS, for each color channel, as shown in FIG. 5C. The chromatic change of the blue color channel, ΔS_{blue}, was used to model the activation and relaxation of the SP-6A.

In light of the foregoing, it should be appreciated that the present invention significantly advances the art by providing a dipropiolate-derivatized spiropyran (SP) mechanophore and thiol-yne-derived stereoelastomers doped with these SP mechanophores that are structurally and functionally improved in a number of ways. While particular embodiments of the invention have been disclosed in detail herein, it should be appreciated that the invention is not limited thereto or thereby inasmuch as variations on the invention herein will be readily appreciated by those of ordinary skill in the art. The scope of the invention shall be appreciated from the claims that follow.

### APPENDIX

### Mechanical, Mechanochromic, and Thermal Data Tables

TABLE 2
Mechanical and thermal properties of 0-SP, 1-SP, and 1.5-SP

| | | | | Mechanical Properties | | | | | | Thermal Properties | | | |
|---|---|---|---|---|---|---|---|---|---|---|---|---|---|
| | SP | cis | M_{w} | \dot{ε} | Ε | λ_{yield} | σ_{yield} | λ_{break} | σ_{break} | T_{g} | T_{c} | Tₘ | ΔHₘ |
| ID | (wt %) | (%) | (kDa) | (mm/min) | (MPa) | (%) | (MPa) | (%) | (MPa) | (° C.) | (° C.) | (° C.) | (J/g) |
| 0-SP | 0 | 80 | 72.6 | 10 | 128.6 ± 7.1 | 14 ± 1 | 8.4 ± 0.2 | 599 ± 21 | 30.0 ± 3.5 | −0.8 | 41.6 | 87.1 | 14.8 |
| | | | | 1 | 115.6 ± 3.4 | 12 ± 1 | 6.3 ± 0.9 | 683 ± 61 | 26.0 ± 2.0 | | | | |
| | | | | 0.1 | 96.9 ± 5.7 | 12 ± 2 | 4.4 ± 0.4 | 751 ± 51 | 22.0 ± 0.8 | | | | |
| 1-SP | 0.98 | 80 | 50.4 | 10 | 130.0 ± 9.0 | 9 ± 3 | 6.9 ± 1.9 | 781 ± 58 | 28.5 ± 2.6 | 0.1 | 36.8 | 91.1 | 16.7 |
| | | | | 1 | 104.3 ± 5.8 | 12 ± 1 | 5.8 ± 0.6 | 826 ± 64 | 26.5 ± 1.9 | | | | |
| | | | | 0.1 | 97.9 ± 2.3 | 13 ± 1 | 6.7 ± 1.6 | 880 ± 38 | 23.3 ± 2.5 | | | | |
| 1.5-SP | 1.47 | 79 | 56.4 | 10 | 106.6 ± 4.0 | 16 ± 0 | 8.5 ± 1.9 | 967 ± 18 | 55.8 ± 2.8 | 2.3 | 40.7 | 86.4 | 14.6 |
| | | | | 1 | 89.3 ± 3.1 | 12 ± 2 | 5.0 ± 1.5 | 995 ± 52 | 46.0 ± 6.2 | | | | |
| | | | | 0.1 | 82.3 ± 4.9 | 16 ± 1 | 5.7 ± 0.6 | 974 ± 39 | 42.9 ± 2.5 | | | | |

Mechanical data were determined by tensile testing and are presented as an average ±1 standard deviation of three independent experiments. Thermal data are reported from DSC 2^{nd} run heating curves (See, FIGS. 8A-F, 9), and T_{g}, Tₘ, T_{c}, and ΔHₘ are reported as the average of 2 experiments. M_{w} determined by SEC analysis. (See, FIGS. 2A-D). % cis was determined by ¹H NMR analysis. (See, FIGS. 68-70). SP content listed is the wt % of monomer 4 used during polymerization.

TABLE 3
Mechanochromism properties in polymers 1-SP and 1.5-SP, and 1-SP_{Reset}

| | | Mechanical Properties | | | | | Mechanochromism | | | |
|---|---|---|---|---|---|---|---|---|---|---|
| | M_{w} | \dot{ε} | Ε | λ_{yield} | σ_{yield} | λ_{break} | σ_{break} | λ_{Act} | σ_{Act} | k |
| Polymer | (kDa) | (mm/min) | (MPa) | (%) | (MPa) | (%) | (MPa) | (%) | (MPa) | (min⁻¹) |
| 1-SP | 50.4 | 10 | 130.0 ± 9.0 | 9 ± 3 | 6.9 ± 1.9 | 781 ± 58 | 28.5 ± 2.6 | 324 ± 10 | 11.6 ± 2.2 | 0.373 ± 0.074 |
| | | 1 | 104.3 ± 5.8 | 12 ± 1 | 5.8 ± 0.6 | 826 ± 64 | 26.5 ± 1.9 | 422 ± 31 | 12.9 ± 1.0 | 0.249 ± 0.014 |
| | | 0.1 | 97.9 ± 2.3 | 13 ± 1 | 6.7 ± 1.6 | 880 ± 38 | 23.3 ± 2.5 | 515 ± 8 | 14.0 ± 1.8 | 0.087 ± 0.017 |
| 1.5-SP | 56.4 | 10 | 106.6 ± 4.0 | 16 ± 0 | 8.5 ± 1.9 | 967 ± 18 | 55.8 ± 2.8 | 338 ± 12 | 14.1 ± 0.7 | 0.488 ± 0.025 |
| | | 1 | 89.3 ± 3.1 | 12 ± 2 | 5.0 ± 1.5 | 995 ± 52 | 46.0 ± 6.2 | 381 ± 11 | 11.8 ± 1.6 | 0.285 ± 0.008 |
| | | 0.1 | 82.3 ± 4.9 | 16 ± 1 | 5.7 ± 0.6 | 974 ± 39 | 42.9 ± 2.5 | 427 ± 10 | 14.8 ± 1.5 | 0.074 ± 0.005 |
| 1-SP_{Reset} | 45.3 | 10 | 114.8 ± 3.1 | 15 ± 2 | 7.2 ± 0.8 | 826 ± 41 | 30.0 ± 2.8 | 382 ± 52 | 13.0 ± 0.5 | 0.280 ± 0.021 |
| | | 1 | 108.3 ± 12.1 | 13 ± 2 | 6.3 ± 0.7 | 684 ± 9 | 22.1 ± 2.2 | 436 ± 38 | 13.5 ± 1.1 | 0.217 ± 0.011 |
| | | 0.1 | 100.7 ± 3.4 | 16 ± 1 | 6.4 ± 0.1 | 687 ± 48 | 20.1 ± 2.2 | 535 ± 24 | 15.4 ± 0.2 | 0.094 ± 0.021 |

Molecular weights were determined by SEC analysis. (See, FIGS. 2A-D). Mechanical data were determined by tensile testing (n = 3). λ_{Act} was calculated according to Section IX (n = 3); the corresponding σ_{Act} were obtained from tensile test data. SP recovery constant k reported for n = 12.

TABLE 4
Crosshead beam speeds, gauge lengths, and strain rates

| | Speed of Crosshead | Avg. Gauge | Nominal Strain | Nominal Strain |
|---|---|---|---|---|
| Polymer | beam (mm min⁻¹) | Length (mm) | Rate (min⁻¹) | Rate (s⁻¹) |
| 0-SP | 10 | 14.3 ± 0.5 | 0.70 ± 0.03 | 0.016 ± 0.001 |
| | 1 | 12.9 ± 0.9 | 0.08 ± 0.01 | 0.0013 ± 0.0001 |
| | 0.1 | 13.1 ± 0.4 | 0.008 ± 0.001 | 0.00013 ± 0.00001 |
| 1-SP | 10 | 14.4 ± 0.6 | 0.70 ± 0.04 | 0.012 ± 0.001 |
| | 1 | 13.2 ± 0.7 | 0.076 ± 0.004 | 0.0013 ± 0.0001 |
| | 0.1 | 12.2 ± 0.6 | 0.0083 ± 0.0011 | 0.00014 ± 0.00002 |
| 1.5-SP | 10 | 11.3 ± 0.2 | 0.91 ± 0.02 | 0.015 ± 0.001 |
| | 1 | 10.7 ± 0.5 | 0.094 ± 0.004 | 0.0015 ± 0.0001 |
| | 0.1 | 11.3 ± 0.4 | 0.0088 ± 0.0074 | 0.00015 ± 0.00001 |
| 1-SP_{Reset} | 10 | 10.7 ± 0.4 | 0.94 ± 0.03 | 0.016 ± 0.001 |
| | 1 | 11.3 ± 0.5 | 0.088 ± 0.004 | 0.0015 ± 0.0001 |
| | 0.1 | 10.9 ± 0.4 | 0.0092 ± 0.0004 | 0.00015 ± 0.00001 |

Mechanical testing conditions averaged from n = 3 experiments.

TABLE 5
Thermal properties of 1-SP and 1-SP_{Reset}

| | T_{g} | T_{c} | ΔH_{c} | Tₘ | ΔHₘ |
|---|---|---|---|---|---|
| Polymer | (° C.) | (° C.) | (J/g) | (° C.) | (J/g) |
| 1-SP | 0.1 | 36.8 | 12.4 | 91.1 | 16.5 |
| 1-SP_{Reset} | −0.1 | 38.9 | 12.1 | 91.1 | 15.8 |

Thermal data are reported from DSC 2^{nd} run heating curves, and all data are reported as the average of 2 experiments.

TABLE 6
Thermal properties confirming strain rate-dependent SIC in 0-SP

| | | \dot{ε} | 1^{st} Heating Cycle | | 2^{nd} Heating Cycle | | | | |
|---|---|---|---|---|---|---|---|---|---|
| Condition | Set* | (mm/min) | Tₘ (° C.) | ΔHₘ (J/g) | T_{g} (° C.) | T_{c} (° C.) | ΔH_{c} (J/g) | Tₘ (° C.) | ΔHₘ (J/g) |
| Control | a | — | 70.4 | 25.9 | −0.9 | 42.4 | 6.8 | 87.0 | 13.0 |
| Stretched | | 10 | 74.5 | 38.8 | −1.1 | 45.4 | 8.0 | 84.2 | 13.0 |
| Stretched | | 1 | 83.4 | 47.2 | −0.9 | 44.8 | 8.2 | 83.0 | 12.6 |
| Control | b | — | 87.6 | 24.9 | −0.5 | 41.8 | 9.7 | 88.1 | 12.4 |
| Stretched | | 10 | 89.2 | 36.5 | −0.6 | 40.6 | 10.1 | 83.1 | 14.1 |
| Stretched | | 1 | 87.3 | 47.3 | −0.5 | 42.9 | 8.9 | 84.2 | 13.0 |
| Avg. % increase | | 10 | 3.8 | 48.4 | 15.6 | 2.1 | 10.9 | −4.4 | 6.9 |
| from Control | | 1 | 9.1 | 86.1 | 0.0 | 4.1 | 6.2 | −4.5 | 0.9 |
| experiments | | | | | | | | | |

*All experiments of the same Set (a or b) were completed on the same day. (See also, FIGS. 4A-F, 8A-F, 10A-F). For stretched samples, the % increase was calculated from their respective control experiments. These percentages were then averaged between each of the two Sets, a and b, and those values are reported at the bottom of the table.

What is claimed is:

1. A dipropiolate-derivatized spiropyran (SP) mechanophore comprising two propriolate functional groups each joined by a C₂-C₁₀ alkyl chain and an ester linkage to a spiropyran molecule.

2. The dipropiolate-derivatized spiropyran (SP) mechanophore of claim 1 wherein said C₂-C₁₀ alkyl chains are C₅H₁₀ alkyl chains.

3. The dipropiolate-derivatized spiropyran (SP) mechanophore of claim 1 wherein said mechanophore exhibits a blue color when under strain.

4. The dipropiolate-derivatized spiropyran (SP) mechanophore of claim 1 having the formula: 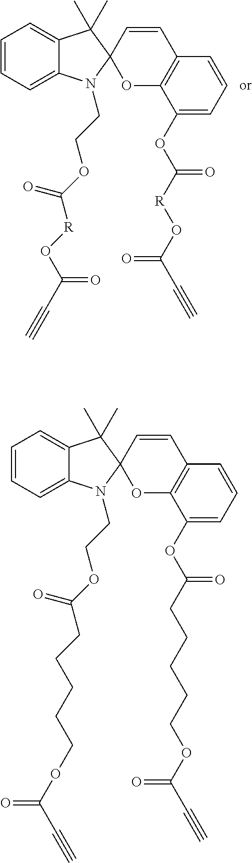
where R is a C₂-C₁₀ alkyl chain.

5. A linear thiol-yne-derived stereoelastomer comprising the dipropiolate-derivatized spiropyran (SP) mechanophore of claim 1 wherein said linear thiol-yne-derived stereoelastomer elastomer exhibits strain induced mechanochromatism.

6. The linear thiol-yne-derived stereoelastomer of claim 5 wherein said linear thiol-yne-derived stereoelastomer comprises a polymer chain containing a plurality of stereoactive alkene bonds wherein 75% or more of these alkene bonds are in a cis orientation.

7. The linear thiol-yne-derived stereoelastomer of claim 6 wherein said stereoelastomer comprises a plurality of stereoactive alkene bonds and 80% or more of these alkene bonds are in a cis orientation.

8. The linear thiol-yne-derived stereoelastomer of claim 5 comprising the residues of two or more a C₂-C₂₀ dithiols and two or more C₂-C₈ dipropiolates.

9. The linear thiol-yne-derived stereoelastomer of claim 8 wherein the ratio of said two or more C₂-C₂₀ dithiols to said two or more C₂-C₈ dipropiolates is about 1:1.

10. The linear thiol-yne-derived stereoelastomer of claim 5 wherein said elastomer exhibits a visible blue color upon strain hardening.

11. The linear thiol-yne-derived stereoelastomer of claim 5 having no chemical crosslinks.

12. The linear thiol-yne-derived stereoelastomer of claim 5 comprising the reaction product of a dipropiolate-derivatized spiropyran (SP) mechanophore, a C₂-C₂₀ dithiol, and a C₂-C₈ dipropiolate.

13. The linear thiol-yne-derived stereoelastomer of claim 5 comprising the reaction product of a dipropiolate-derivatized spiropyran (SP) mechanophore, 1,6-hexanedithiol, and propane-1,3-diyl dipropiolate.

14. The linear thiol-yne-derived stereoelastomer of claim 5 wherein said elastomer comprises from about 001 wt. % to about 5 wt. % of the dipropiolate-derivatized SP mechanophores.

15. The linear thiol-yne-derived stereoelastomer of claim 14 wherein said elastomer comprises from about 0.01 wt. % to about 2 wt. % of said dipropiolate-derivatized SP mechanophores.

16. The linear thiol-yne-derived stereoelastomer of claim 14 wherein said elastomer comprises from about 0.8 wt. % to about 1.6 wt. % of said dipropiolate-derivatized SP mechanophores.

17. The linear thiol-yne-derived stereoelastomer of claim 5 wherein said elastomer has substantially the same mechanical and thermal properties as comparable elastomers not comprising said dipropiolate-derivatized SP mechanophores.

18. The linear thiol-yne-derived stereoelastomer of claim 5 having a Young's modulus of from about 70 MPa to about 150 MPa.

19. The linear thiol-yne-derived stereoelastomer of claim 5 having the same mechanophoric properties after melt recycling.

20. The linear thiol-yne-derived stereoelastomer of claim 5 having the formula: 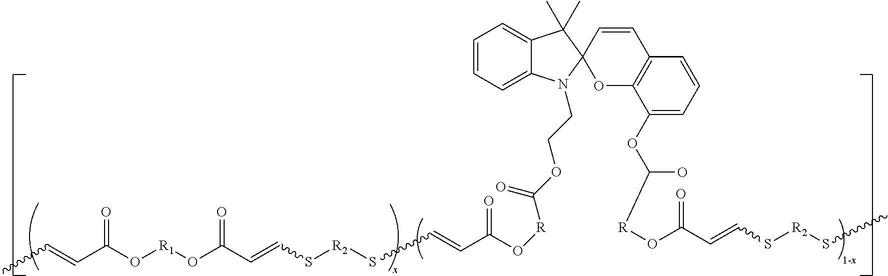
where x is a mole fraction from about 0.95 to about 0.999; R is a C₂-C₁₀ alkyl group; R₁ is a C₂ to C₈ alkyl group; R₂ is a C₂-C₂₀ alkyl group, an aryl group, an ether group, or a poly(ethylene glycol); and [symbol] indicates a stereoactive bond.

21. The linear thiol-yne-derived stereoelastomer of claim 5 having the formula: 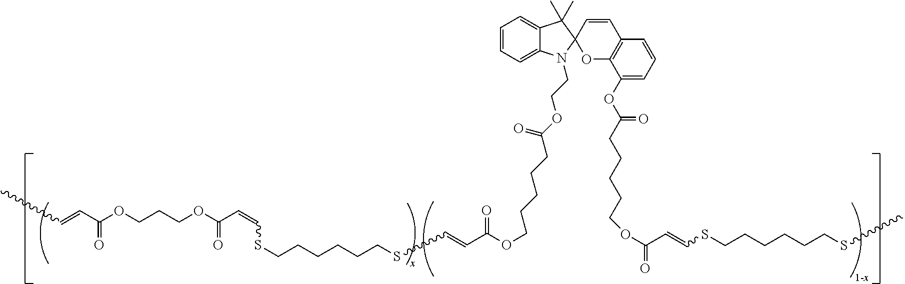
where x is a mole fraction of from about 0.950 to about 0.999.

22. The thiol-yne-derived stereoelastomer of claim 21 wherein x is a mole fraction of from about 0.950 to about 0.999.

23. The thiol-yne-derived stereoelastomer of claim 21 wherein x is a mole fraction of about 0.995.

24. A film comprising the thiol-yne-derived stereoelastomer of claim 5.

25. A method for making the dipropiolate-derivatized spiropyran (SP) mechanophore of claim 1 comprising:
A) dissolving a spiropyran (SP) diol and an organic base in a suitable solvent;
B) adding a quantity of 6-bromohexanoic anhydride to the solution of step A and stirring for from about 6 to about 18 hours to produce 2-(8-((6-bromohexanoyl)oxy)-3′,3′-dimethylspiro [chromene-2,2′-indolin]-1′-yl) ethyl 6-bromohexanoate;
C) placing the 2-(8-((6-bromohexanoyl)oxy)-3′,3′-dimethylspiro [chromene-2,2′-indolin]-1′-yl) ethyl 6-bromohexanoate of step B in a suitable reaction vessel in a dark location and adding sodium propiolate;
D) sealing the reaction vessel and heating it to a a curing temperature until cured;
E) collecting and purifying the reaction product of step D to produce the 3′,3′-dimethyl-1′-(2-((6-(propioloyloxy)hexanoyl) oxy)ethyl) spiro[chromene-2,2′-indolin]-8-yl 6-(propioloyloxy)hexanoate.

26. The method of claim 25 wherein said spiropyran (SP) diol has the formula:

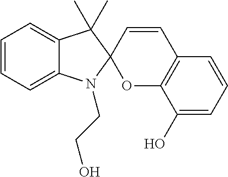 [SMILES: CC1(C)c2ccccc2N(CCO)C12C=Cc1cccc(O)c1O2]

27. The method of claim 25 wherein said organic base is N,N′-dimethylamino pyridine (DMAP).

28. A method for making a thiol-yne-derived stereoelastomer comprising:
A) combining dipropiolate-derivatized spiropyran (SP) mechanophore of claim 4, a C₂-C₂₀ dithiol, and a first quantity of a C₂-C₈ dipropiolate in a suitable reaction vessel;
B) cooling the reaction vessel to a temperature of from about −20° C. to about −5° C. and stirring for from about 14 min to about 60 min;
C) separately combining 1,8-diazabicyclo[5.4.0]undec-7-ene (DBU) and a polar solvent or solvent combination and slowly adding the combination to the reaction vessel;
D) allowing the reaction vessel to warm to room temperature and after from about 15 to about 45 min, dissolving a second quantity of the C₂-C₈ dipropiolate in a suitable solvent;
E) adding the C₂-C₈ dipropiolate solution of step D to the reaction vessel to end-cap any unreacted thiol groups;
F) adding a radical scavenger to the reaction vessel to prevent crosslinking;
G) collecting a purifying the reaction product of step F to produce the thiol-yne-derived stereoelastomer of claim 5.

29. The method of claim 28 wherein said C₂-C₂₀ dithiol in step A is 1,6-hexanedithiol.

30. The method of claim 28 wherein said C₂-C₈ dipropiolate in steps A and E is propane-1,3-diyl dipropiolate.

31. The method of claim 28 wherein the step of slowly adding (step C) comprises adding to the DBU solution dropwise over a period of about 20 min.
